(19) United States
(12) Patent Application Publication
(10) Pub. No.: US 2026/0184811 A1
(43) Pub. Date: Jul. 2, 2026

(54) HER2-BINDING AGENTS AND USES THEREOF

(71) Applicant: Cereius, Inc., Durham, NC (US)

(72) Inventors: Jeffrey L. SCHAAL, Durham, NC (US); Robin MARJORAM, Durham, NC (US)

(73) Assignee: Cereius, Inc., Durham, NC (US)

(21) Appl. No.: 18/868,284

(22) PCT Filed: May 23, 2023
(86) PCT No.: PCT/US2023/067376

§ 371 Date: Nov. 22, 2024

Related U.S. Application Data
(60) Provisional application No. 63/344,808, filed on May 23, 2022.

(51) Int. Cl. C07K 16/32; A61K 51/10; A61P 35/00; G01N 33/575

(52) U.S. Cl. CPC C07K 16/32; A61K 51/103; A61K 51/1096; A61P 35/00; G01N 33/5759; A61K 2123/00; C07K 2317/569

(57) ABSTRACT

Provided are HER2-binding agents comprising a VHH domain that specifically binds HER2. Nucleic acids encoding the proteins, vectors and host cells comprising the nucleic acids, and methods of making and using the HER2-binding agents are also provided. The disclosure also provides HER2-binding agent conjugates wherein the protein is conjugated to a detectable label and HER2-binding targeted radiotherapeutic agents for therapeutic and/or diagnostic purposes. Methods of using such HER2-binding agents to detect, monitor, and/or treat diseases and conditions such as cancer are also provided.

### CROSS-REFERENCE TO RELATED APPLICATION

This application claims priority from U.S. Provisional Patent Application 63/344,808, filed May 23, 2022. The disclosure of that priority application is incorporated by reference herein in its entirety.

### SEQUENCE LISTING

The instant application contains a Sequence Listing which has been submitted electronically in XML format and is hereby incorporated by reference herein in its entirety. The electronic copy of the Sequence Listing, created on May 23, 2023, is named 122878.WO012.xml and is 271,281 bytes in size.

### BACKGROUND OF THE INVENTION

Major progress has been achieved in developing and optimizing HER2-targeted therapies for breast cancer. Examples of FDA-approved drugs with significant clinical efficacy for metastatic disease include the anti-HER2 antibody trastuzumab, the anti-HER2-HER3 dimerization inhibitor pertuzumab, and the antibody-drug conjugate T-DM1 (Krop et al., Lancet Oncol. (2014) 15(7):689-99; Slamon et al., NEJM (2001) 344:783-92). However, many limitations and resistances impair the efficacy of these targeted therapeutics in HER2⁺ breast cancer, and they are not suited for treating the majority of breast cancers in which HER2 is weakly over-expressed.

Brain metastasis is a devastating progression of breast cancer that has a reported incidence as high as 30-55% in HER2-positive breast cancer patients. Brain metastasis is a significant source of morbidity for HER2-positive breast cancer patients with up to 50% of the patients dying from the direct consequences of CNS disease progression. Treatment options for HER2⁺ breast cancer brain metastases (BCBM) are limited, and the same anti-HER2 therapies that slow growth systemically do not typically control brain metastases due to the inadequate penetration of antibodies through the blood-brain barrier (BBB) (Lampson, mAbs (2011) 3:153-60. Additionally, clinical data reveal an increased incidence of HER2-positive breast cancer brain metastases after adjuvant trastuzumab therapy (Olson et al., Ann Oncol. (2013) 24:1526-33.

Moreover, despite adequate drug delivery, the efficacy of small molecules on breast cancer brain metastases is also very limited and can only be marginally increased through the addition of further therapeutic modalities (Lin et al., J Clin Oncol. (2008) 26:1993-9; Lin et al., Clin Cancer Res. (2009) 15:1452-9; Bachelot et al., Lancet Oncol. (2013) 14: 64-71).

Hyperproliferative disorders such as cancers characterized by overexpression of HER2 (called HER2⁺ cancers) are often poor in prognosis or resistant to many standard therapies. Thus, there is a need for new therapies that are effective in the treatment of cancers such as HER2-positive cancers and metastatic HER2-positive cancers. The present disclosure satisfies these needs and provides other advantages as well, including the ability to selectively deliver HER2-targeted agents to HER2-positive tumors in the central nervous system, without compromising normal brain tissue.

### SUMMARY OF THE INVENTION

The disclosure provides novel HER2 (human epidermal growth factor receptor 2) binding VHH domains and addresses the problems associated with the treatment of HER2⁺ cancer, including HER2⁺ cancer that has metastasized to the brain. The disclosure provides radiolabeled targeted HER2-binding agents that are capable of being taken up and retained by the tumor cells while reducing the amount of the radionuclides that are taken up by normal cells, particularly the kidneys, which has been found to be the biggest toxicity limiting concern for targeted radiotherapeutics. The disclosure includes HER2-binding agents that are radiolabeled with radioactive halogen atoms in a manner which minimizes loss of the radioactive halogen due to dehalogenation in vivo and has been found to preserve the biological activity of the HER2-binding VHH domain, maximize retention in diseased cells such as cancer cells, and minimize the retention of radioactivity in normal tissues after in vivo administration.

The disclosure provides HER2-binding agents comprising a VHH domain that specifically binds HER2. Nucleic acids encoding the HER2-binding agents, vectors and host cells comprising the nucleic acids, and methods of making and using the HER2-binding agents are also provided. The disclosure also provides HER2-binding agents that contain a detectable label as well as HER2-binding agent targeted radiotherapeutic agents for therapeutic and/or diagnostic purposes. Methods of using the HER2-binding agents to detect, monitor, and/or treat diseases and conditions such as cancer are also provided.

In some embodiments, the disclosure is directed to:
[1] a HER2-binding agent comprising a HER2-binding VHH domain that comprises:
(a) a complementarity determining region (CDR) 1 comprising an amino acid sequence selected from SEQ ID NOs: 82-101; a CDR2 comprising an amino acid sequence selected from SEQ ID NOs: 102-123; and a CDR3 comprising an amino acid sequence selected from SEQ ID NOs: 124-157 and 206;
(b) a CDR1, CDR2 and CDR3 comprising the amino acid sequence of a CDR1, CDR2 and CDR3 contained in a VHH sequence disclosed in Table 2;
(c) a VHH amino acid sequence disclosed in Table 2;
(d) a VHH sequence selected from SEQ ID NOs: 1-81 and 205;
(e) an amino acid sequence having at least 80%, 85%, 90%, 95%, 96%, 97%, 98% or 99% sequence identity with a VHH sequence disclosed in Table 2;
(f) an amino acid sequence having at least 80%, 85%, 90%, 95%, 96%, 97%, 98% or 99% sequence identity with a VHH sequence selected from SEQ ID NOs: 1-81 and 205;
(g) an amino acid sequence that has 1-25, 1-20, 1-15, 1-10, 1-5, or 1-3 additions, substitutions or deletions compared to a reference VHH sequence disclosed in Table 2; or
(h) an amino acid sequence that has 1-25, 1-20, 1-15, 1-10, 1-5, or 1-3 additions, substitutions or deletions compared to a reference VHH sequence selected from SEQ ID NOs: 1-81 and 205;
[2] the HER2-binding agent of [1], wherein the VHH domain comprises:
(a) a CDR1 comprising SEQ ID NO: 101; a CDR2 comprising SEQ ID NO: 111; and a CDR3 comprising SEQ ID NO: 135 or 136;
(b) a CDR1 comprising SEQ ID NO: 101; a CDR2 comprising SEQ ID NO: 111; and a CDR3 comprising SEQ ID NO: 135;
(c) a CDR1 comprising SEQ ID NO: 101; a CDR2 comprising SEQ ID NO: 111; and a CDR3 comprising SEQ ID NO: 136;
(d) a CDR1 comprising SEQ ID NO: 82; a CDR2 comprising SEQ ID NO: 102; and a CDR3 comprising SEQ ID NO: 124, 145, 146 or 147;
(e) a CDR1 comprising SEQ ID NO: 83; a CDR2 comprising SEQ ID NO: 103; and a CDR3 comprising SEQ ID NO: 125;
(f) a CDR1 comprising SEQ ID NO: 84; a CDR2 comprising SEQ ID NO: 104; and a CDR3 comprising SEQ ID NO: 126;
(g) a CDR1 comprising SEQ ID NO: 85; a CDR2 comprising SEQ ID NO: 105; and a CDR3 comprising SEQ ID NO: 127;
(h) a CDR1 comprising SEQ ID NO: 86; a CDR2 comprising SEQ ID NO: 106; and a CDR3 comprising SEQ ID NO: 128, 130, 134 or 155;
(i) a CDR1 comprising SEQ ID NO: 87; a CDR2 comprising SEQ ID NO: 117; and a CDR3 comprising SEQ ID NO: 129;
(j) a CDR1 comprising SEQ ID NO: 88; a CDR2 comprising SEQ ID NO: 118; and a CDR3 comprising SEQ ID NO: 130;
(k) a CDR1 comprising SEQ ID NO: 89; a CDR2 comprising SEQ ID NO: 108 or 112; and a CDR3 comprising SEQ ID NO: 131 or 157;
(l) a CDR1 comprising SEQ ID NO: 89; a CDR2 comprising SEQ ID NO: 108; and a CDR3 comprising SEQ ID NO: 131;
(m) a CDR1 comprising SEQ ID NO: 89; a CDR2 comprising SEQ ID NO: 108 or 112; and a CDR3 comprising SEQ ID NO: 156;
(n) a CDR1 comprising SEQ ID NO: 89; a CDR2 comprising SEQ ID NO: 108 or 112; and a CDR3 comprising SEQ ID NO: 157;
(o) a CDR1 comprising SEQ ID NO: 90; a CDR2 comprising SEQ ID NO: 109; and a CDR3 comprising SEQ ID NO: 132;
(p) a CDR1 comprising SEQ ID NO: 91; a CDR2 comprising SEQ ID NO: 119; and a CDR3 comprising SEQ ID NO: 137;
(q) a CDR1 comprising SEQ ID NO: 92; a CDR2 comprising SEQ ID NO: 120; and a CDR3 comprising SEQ ID NO: 138;
(r) a CDR1 comprising SEQ ID NO: 93; a CDR2 comprising SEQ ID NO: 113 or 114; and a CDR3 comprising SEQ ID NO: 139;
(s) a CDR1 comprising SEQ ID NO: 93, a CDR2 comprising SEQ ID NO: 114, and a CDR3 comprising SEQ ID NO: 206;
(t) a CDR1 comprising SEQ ID NO: 94; a CDR2 comprising SEQ ID NO: 115; and a CDR3 comprising SEQ ID NO: 140 or 149;
(u) a CDR1 comprising SEQ ID NO: 95; a CDR2 comprising SEQ ID NO: 121; and a CDR3 comprising SEQ ID NO: 141;
(v) a CDR1 comprising SEQ ID NO: 96; a CDR2 comprising SEQ ID NO: 122; and a CDR3 comprising SEQ ID NO: 142;
(w) a CDR1 comprising SEQ ID NO: 97; a CDR2 comprising SEQ ID NO: 116; and a CDR3 comprising SEQ ID NO: 150;
(x) a CDR1 comprising SEQ ID NO: 98; a CDR2 comprising SEQ ID NO: 107; and a CDR3 comprising SEQ ID NO: 144, 151, 152 or 153;
(y) a CDR1 comprising SEQ ID NO: 99; a CDR2 comprising SEQ ID NO: 109; and a CDR3 comprising SEQ ID NO: 132 or 148; or
(z) a CDR1 comprising SEQ ID NO: 100; a CDR2 comprising SEQ ID NO: 110; and a CDR3 comprising SEQ ID NO: 133 or 154; or
the HER2-binding agent of [1], wherein the VHH domain comprises:
(aa) a CDR1 comprising SEQ ID NO: 97; a CDR2 comprising SEQ ID NO: 123; and a CDR3 comprising SEQ ID NO: 143; or
(ab) a CDR1 comprising SEQ ID NO: 101; a CDR2 comprising SEQ ID NO: 111; and a CDR3 comprising SEQ ID NO: 136.
[3] the HER2-binding agent of [1] or [2], wherein said HER2-binding agent is monovalent;
[4] the HER2-binding agent of [1] or [2], wherein said HER2-binding agent is multivalent (e.g., bivalent, trivalent, or tetravalent);
[5] the HER2-binding agent of any one of [1]-[4], wherein said HER2-binding agent is monospecific;
[6] the HER2-binding agent of any one of [1], [2], or [4], wherein said HER2-binding agent is multispecific and specifically binds HER2 and a different antigen;
[7] the HER2-binding agent of any one of [1]-[6], wherein the VHH domain is humanized;
[8] the HER2-binding agent of any one of [1]-[7], wherein said HER2-binding agent comprises a fusion protein comprising the HER2 binding VHH domain and a heterologous sequence;
[9] the HER2-binding agent of any one of [1]-[8], wherein said HER2-binding agent comprises an N-terminal amino acid residue or polypeptide sequence, optionally wherein the N-terminal residue or sequence improves protein expression, solubility, purification, half-life, or avidity, such as a Met-Ala or an Ala leader peptide;
[10] the HER2-binding agent of any one of [1]-[9], wherein said HER2-binding agent comprises a C-terminal amino acid residue or polypeptide sequence, optionally wherein the C-terminal residue or sequence facilitates radiolabeling selectivity, loading, cell internalization, half-life, or avidity;
[11] The HER2-binding agent of any one of [1]-[10], wherein said HER2-binding agent comprises a polypeptide sequence having the structure:

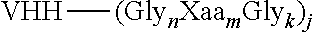

[i] where VHH is a VHH disclosed in Table 2, or elsewhere herein (e.g., a VHH selected from SEQ ID NOs: 1-81 and 205),
[ii] n=0-5,
[iii] m=0-5,
[iv] k=0-6,
[v] j=0-8, and
[vi] Xaa is an amino acid that enables site conjugation to radiolabeled prosthetic groups, or direct radiolabeling;
[12] the HER2-binding agent of any one of [8]-[11], wherein the fusion protein comprises an Fc region;
[13] the HER2-binding agent of any one of [8]-[11], wherein the fusion protein is a chimeric antigen receptor (CAR);
[14] an isolated nucleic acid encoding the HER2-binding agent of any one of [1]-[13];
[15] a vector comprising the nucleic acid of [14], optionally wherein the vector is an expression vector;
[16] a host cell comprising the nucleic acid of [14] or the vector of [15];
[17] the host cell of [16], wherein the host cell is a eukaryotic cell such as a mammalian cell or yeast cell, or a prokaryotic cell;
[18] the HER2-binding agent of any one of [1]-[13], wherein said HER2-binding agent is conjugated to a label;
[19] the HER2-binding agent of [18], wherein the label is a fluorescent dye, a radionuclide, an enzyme, a toxin, or a chemotherapeutic agent;
[20] the HER2-binding agent of [18] or [19], wherein the label is a radionuclide;
[21] the HER2-binding agent of [19] or [20], wherein the radionuclide is a radioactive halogen isotope;
[22] the HER2-binding agent of [19] or [20], wherein the radionuclide is a radioactive halogen isotope selected from:
   a) ¹⁸F, ⁷⁶Br, ¹²³I, ¹²⁴I, ¹²⁵I, and ¹³¹I or
   b) ⁷⁵Br, ⁷⁷Br, ¹²²I, ¹²⁴I, ¹²⁵I, ¹³¹I, and ²¹¹At;
[23] the HER2-binding agent of [19] or [20], wherein the radionuclide is a radioactive metal isotope;
[24] the HER2-binding agent of [19] or [20], wherein the radionuclide is a radioactive metal isotope selected from:
   a) ⁴⁴Sc, ⁴⁵Ti, ⁵¹Cr, ⁶²Cu, ⁶⁴Cu, ⁶⁶Ga, ⁶⁸Ga, ⁶⁸Ge, ⁷⁵Se, ⁸²Sr, ⁸⁶Y, ⁹⁹Mo, ^{99m}Tc, ^{110m}In, ¹¹¹In, ¹⁶⁶Ho, ¹⁸⁶Re, ^{195m}Pt, and ²⁰¹Tl; or
   b) ⁴⁷Sc, ⁵²Mn, ⁶⁴Cu, ⁶⁷Cu, ⁶⁷Ga, ⁸⁹Zr, ⁹⁰Y, ¹¹¹In, ¹⁵³Sm, ¹⁴⁹Tb, ¹⁶¹Tb, ¹⁶⁶Ho, ¹⁷⁷Lu, ¹⁸⁸Re, ²¹²Pb, ²¹²Bi, ²¹³Bi, ²²⁵Ac, ²²⁶Th, and ²²⁷Th;
[25] the HER2-binding agent of any one of [1]-[13] and [18]-[24], wherein said HER2-binding agent is conjugated to a label via a chelating moiety;
[26] the HER2-binding agent of [25], wherein the chelating moiety is covalently linked to the protein via a lysine or cysteine residue;
[27] the HER2-binding agent of [25] or [26], wherein the label forms a complex with a metal, and wherein the complex is chelated by the chelating moiety;
[28] the HER2-binding agent of any one of [1]-[12] and [18]-[27], wherein said HER2-binding agent is a targeted radiotherapeutic agent having the structure:

VHH-RLC,
wherein
VHH is a protein comprising a HER2-binding VHH domain provided herein (e.g., having a VHH amino acid sequence as shown in Table 2, e.g., a VHH comprising an amino acid sequence selected from SEQ ID NOs: 1-81 and 205, or a derivative thereof), and
RLC is a radiolabeling chemistry used to directly or indirectly facilitate attachment of a radionuclide to the VHH;
[29] the HER2-binding agent of [28], wherein the targeted radiotherapeutic agent comprises a prosthetic compound or radiohalogen precursor represented by Formula 2:

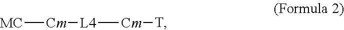

wherein:
MC is a polydentate metal chelating moiety;
Cm is thiourea, amide, or thioether;
L4 is selected from a bond, a substituted or unsubstituted alkyl chain, a substituted or unsubstituted alkenyl chain, a substituted or unsubstituted alkynyl chain optionally having NH, CO, or S on one or both termini, and a polyethylene glycol (PEG) chain;
T is the compound of any of:
(a) a compound in the form of a prosthetic compound or radiohalogen precursor represented by Formula 1:

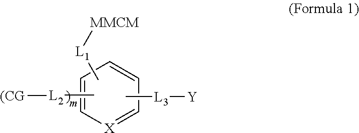

wherein:
X is CH or N;
L1 and L3 are independently selected from a bond, a substituted or unsubstituted alkyl chain, a substituted or unsubstituted alkenyl chain, a substituted or unsubstituted alkynyl chain, and a polyethylene glycol (PEG) chain;
MMCM is a macromolecule conjugating moiety;
L2 is a substituted or unsubstituted alkyl chain, a substituted or unsubstituted alkenyl chain, a substituted or unsubstituted alkynyl chain, or a polyethylene glycol (PEG) chain comprising at least three oxygen atoms, wherein L2 optionally contains a Brush Border enzyme-cleavable peptide;
CG is selected from guanidine; PO3H; SO3H; one or more charged D- or L-amino acids selected from arginine, phosphono/sulfo phenylalanine, glutamate, aspartate, and lysine; a hydrophilic carbohydrate moiety; a polyethylene glycol (PEG) chain; and Z-guanidine;
Z is (CH₂)ₙ;
n is greater than 1;
m is 0 to 3; and
Y is an alkyl metal moiety, boronic acid moiety, boronic ester moiety, or a radioactive halogen selected from the group consisting of ¹⁸F, ⁷⁵Br, ⁷⁶Br, ⁷⁷Br, ¹²²I, ¹²³I, ¹²⁴I, ¹²⁵I, ¹³¹I, and ²¹¹At, or a pharmaceutically acceptable salt or solvate thereof;
(b) the compound of (a), wherein the compound is a radiohalogen precursor, optionally wherein Y is an alkyl metal moiety selected from the group consisting of trimethyl stannyl (SnMe3), tri-n-butylstannyl (SnBu3), trimethylsilyl (SiMe3), a boronic acid (B(OH)₂), or a boronic ester (B(OR)₂), and optionally wherein R comprises a cyclic moiety or an aliphatic group;
(c) the compound of (a), wherein the compound is a prosthetic compound, and wherein Y is a radioactive halogen selected from the group consisting of ¹⁸F, ⁷⁵Br, ⁷⁶Br, ⁷⁷Br, ¹²²I, ¹²³I, ¹²⁴I, ¹²⁵I, ¹³¹I, and ²¹¹At;
(d) the compound of (a), wherein MMCM is an active ester or (Gly)m, wherein m is 1 or more;
(e) the compound of (a), wherein MMCM is selected from the group consisting of N-hydroxysuccinimide (NHS) ester, tetrafluorophenol (TFP) ester, pentafluorophenol (PFP), paranitrolphenol (PNP), an isothiocyanate group, or a maleimide group;
(f) the compound of (a), wherein MMCM is Gly-Gly-Gly;
(g) the compound of (a), wherein, wherein L2 is (CH₂)ₚ, wherein p=1 to 6;
(h) the compound of (a), wherein the optional Brush Border enzyme-cleavable peptide is selected from the group consisting of Gly-Lys, Gly-Tyr and Gly-Phe-Lys;
(i) the compound of (a), represented by the following structure:

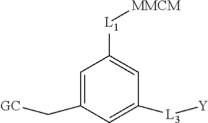

(j) the compound of (i), wherein the compound comprises N-succinimidyl 3-guanidinomethyl-5-[¹³¹I]iodobenzoate (isoSGMIB), or N-succinimidyl 3-[²¹¹At]astato-5-guanidinomethyl benzoate (isoSAGMB), or maleimidoethyl 2-(guanidinomethyl)-5-iodobenzoate (isoMEGMIB), or maleimidoethyl 3-(guanidinomethyl)-5-astatobenzoate (isoMEGMAB);
or a pharmaceutically acceptable salt or solvate thereof;
[30] the HER2-binding agent of [29], wherein MC is a macrocyclic structure;
[31] the HER2-binding agent of [30], wherein MC is selected from DOTA, TETA, NOTP, and NOTA;
[32] the HER2-binding agent of [30], wherein MC is an acyclic polydentate ligand;
[33] the HER2-binding agent of [30], wherein MC is selected from EDTA, EDTMP, and DTPA;
[34] the HER2-binding agent of [30], wherein the compound is a radiohalogen precursor, optionally wherein Y is an alkyl metal moiety selected from the group consisting of trimethyl stannyl (SnMe₃), tri-n-butylstannyl (SnBu₃), trimethylsilyl (SiMe₃), a boronic acid (B(OH)₂), or a boronic ester (B(OR)₂), and optionally wherein R comprises a cyclic moiety or an aliphatic group;
[35] the HER2-binding agent of [30], wherein the compound is a prosthetic compound, and wherein Y is a radioactive halogen selected from ¹⁸F, ⁷⁵Br, ⁷⁶Br, ⁷⁷Br, ¹²¹I, ¹²³I, ¹²⁴I, ¹²⁵I, ¹³¹I, and ²¹¹At;
[36] the HER2-binding agent of [30], further comprising a metal associated with the MC;
[37] the HER2-binding agent of [30], wherein the metal is a radioactive metal selected from: ¹⁷⁷Lu, ⁶⁴Cu, ⁶⁷Cu, ¹¹¹In, ⁹⁰Y, ²²⁵Ac, ²¹²Bi, ²¹³Bi, ¹⁵³Sm, ¹⁶⁶Ho, ²¹²Pb, ²¹²Bi, ⁶⁷Ga ⁶⁸Ga, ⁸⁹Zr, and ²²⁷Th;
[38] a pharmaceutical composition comprising the HER2-binding agent of any one of [1]-[13] and [18]-[37] and a pharmaceutically acceptable carrier;
[39] a method of treating a disease in a subject comprising administering a therapeutically effective amount of the HER2-binding agent of any one of [1]-[13] and [18]-[37], or a pharmaceutical composition of [38], to a subject in need thereof,
[40] the method of [39], wherein the disease is cancer.
[41] a method of treating cancer comprising administering an effective amount of the HER2-binding agent of any one of [1]-[13] and [18]-[37], or a pharmaceutical composition of [38], to a subject in need thereof,
[42] a method of treating cancer comprising administering an effective amount of the targeted radiotherapeutic agent of any one of [28]-[37] to a subject in need thereof;
[43] the method of any one of [40]-[42] wherein the cancer is HER2⁺;
[44] a method of treating a HER2⁺ cancer comprising administering an effective amount of the HER2-binding agent of any one of [1]-[13] and [18]-[37], or a pharmaceutical composition of [38], to a subject in need thereof;
[45] the method of any one of [40]-[44], wherein the cancer is selected from: breast cancer, ovarian cancer, cervical cancer, endometrial cancer, head and neck cancer, brain cancer, gastric cancer (e.g., colorectal and colon cancer), pancreatic cancer, renal cancer, prostate cancer, lung cancer (e.g., NSCLC), bladder cancer, urothelial cancer, and an epidermal-derived cancer, and/or metastase(s) originating therefrom;
[46] the method of any one of [40]-[45], wherein the cancer is breast cancer and/or a metastasis originating therefrom;
[47] the method of [46], wherein the cancer is a breast cancer brain metastasis;
[48] the method of any one of [40]-[47], wherein the subject is scored HER2 (2+) or HER2 (3+) by immunohistochemistry;
[49] a method of killing a HER2⁺ cell comprising contacting the cell with a HER2-binding agent of any one of [1]-[13] and [18]-[37], or a pharmaceutical composition of [38];
[50] the method of [49], wherein the cell is a cancer cell;
[51] the method of [50] wherein the cancer cell is selected from: breast cancer, ovarian cancer, cervical cancer, endometrial cancer, head and neck cancer, brain cancer, gastric cancer (e.g., colorectal and colon cancer), pancreatic cancer, renal cancer, prostate cancer, lung cancer (e.g., NSCLC), bladder cancer, urothelial cancer, and an epidermal-derived cancer, and/or a metastasis originating therefrom;
[52] the method of any one of [49]-[51], wherein the cell is contacted with the HER2-binding agent in vitro or in vivo;
[53] a HER2-binding agent of any one of [1]-[13] and [18]-[37], or a pharmaceutical composition of [38], for use in medicine (e.g., in a method of any one of [39]-[52]);
[54] use of the HER2-binding agent of any one of [1]-[13] and [18]-[37], or the pharmaceutical composition of [38], in the manufacture of a medicament for the treatment of a disease in a subject in need thereof, e.g., in a method of any one of [39]-[52]);
[55] the use of [54], wherein the disease is cancer;
[56] a method of diagnosing a disease or medical condition in a subject, comprising administering the HER2-binding agent of any one of [1]-[13] and [18]-[37], or the pharmaceutical composition of [38], to the subject;
[57] the method of [56], wherein the disease is cancer;
[58] a method of detecting HER2⁺ cells in a subject, comprising:
   a) administering a detectably labeled HER2-binding agent of any one of [1]-13] and [18]-[37], or the pharmaceutical composition of [38] in which the HER2-binding agent has been detectably labeled, to the subject; and
   b) detecting binding of the labeled HER2-binding agent to HER2⁺ cells in the subject, wherein the detection of the binding indicates the presence of HER2⁺ cells;
[59] the method of [58], wherein detecting binding of the labeled HER2-binding agent to HER2⁺ cells in the subject comprises imaging HER2⁺ cells in the subject;
[60] a method of imaging HER2⁺ cells in a subject, comprising administering a detectably labeled HER2-binding agent of any one of [1]-[13] and [18]-[37], or a pharmaceutical composition of [38] in which the HER2-binding agent has been detectably labeled, to the subject;
[61] the method of [60], wherein imaging HER2⁺ cells in the subject comprises performing a positron emission tomography (PET) scan or positron emission tomography/computed tomography (PET/CT) scan on the subject;
[62] the method of any one of [58]-[61], wherein the HER2⁺ cells are HER2⁺ cancer cells;
[63] the method of any one of [39]-[48] and [54]-[62], wherein the subject is a mammal, e.g., a human or a non-human primate;
[64] the method of any one of [39]-[48] and [54]-[63], wherein the subject has cancer;
[65] a method of making a HER2-binding agent, comprising:
   (a) culturing the host cell of [16] or [17] under conditions where the HER2-binding agent is produced; and
   (b) recovering the HER2-binding agent produced by the host cell.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIGS. 1A-1J are a series of graphs depicting full ELISA affinity binding curves of exemplary VHH sequences against the human HER2 receptor, after protein expression and purification.

FIGS. 2A-2I are a series of graphs depicting specificity of exemplary VHH proteins to other receptors within the ErbB family.

FIGS. 3A-3E are a series of graphs depicting cross-reactivity of exemplary VHH proteins to cross-species HER2 receptors found in other animals.

FIGS. 4A-4B are a pair of graphs depicting competitive inhibition of exemplary VHH proteins with (A) trastuzumab or (B) pertuzumab in an indirect ELISA assay format.

FIGS. 5A-5E relate to in vitro cell assays for evaluating VHH_128 after being radiolabeled with [¹³¹I]-isoSGMIB. (A) Radiolabeled structure. (B) Affinity to SKOV-3 ovarian cancer cells. Levels of [¹³¹I]-isoSGMIB-VHH128 that are (C) surface bound or (D) internalized within SKOV-3 cells after a 2-hour incubation. (E) Measure of [¹³¹I] internalization in SKOV-3 cells in presence of 100-fold trastuzumab.

FIG. 6 is a series of graphs depicting tissue accumulation levels over 24 hours for [¹³¹I]-isoSGMIB-VHH128 in a SKOV-3 xenograft tumor model in female athymic mice.

FIG. 7 is a series of graphs depicting tissue accumulation levels over 24 hours for [¹³¹I]-isoSGMIB-VHH128 in a BT-474 xenograft tumor model in female NOD SCID mice.

FIGS. 8A-8C relate to HER2 targeted alpha-particle therapy utilizing ²¹¹Astatine labeled VHH. (A) Scheme and structure for labeling VHH_128 with ²¹¹At using isoSAGMB. (B) Affinity binding curve for [²¹¹At]-isoSAGMB-VHH128 against the HER2-expressing BT-474 cells. (C) Accumulation levels in BT-474 tumors xenografted in athymic mice.

FIGS. 9A-9C relate to radiolabeling of HER2-targeted VHH using maleimide-based radiolabeling chemistry. (A) Scheme and structure for labeling VHH_1041 with ¹³¹I using isoMEGMIB. (B) Affinity binding curve for [¹³¹I]-isoMEGMIB-VHH141 against the HER2-expressing BT-474 cells. (C) In vitro cellular binding and internalization ratios on BT-474 cells; E: efflux, S: surface bound, I: internalized.

FIG. 10 is a series of graphs depicting tissue accumulation levels over 48 hours for [¹³¹I]-isoMEGMIB-VHH141 in SKOV-3 xenograft tumors in female athymic mice.

### DETAILED DESCRIPTION OF THE INVENTION

Provided herein are HER2-binding agents comprising a VHH domain that specifically binds HER2. Nucleic acids encoding the proteins, vectors and host cells comprising the nucleic acids, and methods of making and using the HER2-binding agents are also provided. The disclosure also provides HER2-binding agents that contain a detectable label and HER2-binding agent targeted radiotherapeutic agents for therapeutic and/or diagnostic purposes. Methods of using the HER2-binding agents to detect, monitor, and/or treat diseases and conditions such as cancer are also provided.

In particular embodiments, the disclosure is directed to HER2-targeted radiotherapeutic agents. The provided targeted radiotherapeutic agents display favorable properties over conventional radiotherapeutic agents that include greater radionuclide stabilization in blood circulation, lower uptake and dose exposure to normal tissues including the kidneys, higher binding affinity and cell internalization, greater tumor uptake in HER2⁺ tumors, and higher maximum tolerable doses for systemic targeted radiotherapeutic delivery of radionuclides including ¹³¹Iodine and ²¹¹Astatine. Other features and advantages of the disclosed compositions and methods will be apparent from the following disclosure, drawings, and claims.

### Definitions

Unless otherwise defined, all technical and scientific terms used herein have the same meaning as commonly understood by one of ordinary skill in the art to which this disclosure pertains. Although methods and materials similar or equivalent to those described herein can be used in the practice or testing of the provided compositions, suitable methods and materials are described herein. Each publication, patent application, patent, and other reference mentioned in this disclosure is herein incorporated by reference in its entirety. In case of conflict, the present specification, including definitions, will control. In addition, the materials, methods, and examples are illustrative only and are not intended to be limiting.

It is understood that wherever embodiments are provided herein with the language “comprising,” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” is considered more of an open-ended phrase while “consisting of” is more exclusive and “consisting essentially of” achieves a middle ground).

As used herein, the singular forms “a”, “an”, and “the” include plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “approximately” and “about,” as applied to one or more values of interest, refer to a value that is similar to a stated reference value. In some embodiments, the terms “about” or “approximately” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of percent amino acid sequence identity to a reference polypeptide sequence, “about” may mean +/−10% of the recited value.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Where embodiments of the disclosure are described in terms of a Markush group or other grouping of alternatives, the disclosed composition or method encompasses not only the entire group listed as a whole, but also each member of the group individually and all possible subgroups of the main group, and also the main group absent one or more of the group members. The disclosed compositions and methods also envisage the explicit exclusion of one or more of any of the group members in the disclosed compositions or methods.

The terms “isolated” and “purified,” or iterations thereof, as used herein refer to a molecule that has been separated from at least some of the components with which it is typically found in nature or produced. For example, a protein is referred to as “isolated” when it is separated from at least some of the components of the cell in which it was produced. Where a protein is secreted by a cell after expression, physically separating the supernatant containing the protein from the cell that produced it is considered to be “isolating” the protein. Similarly, a nucleic acid is referred to as “isolated” when it is not part of the larger nucleic acid (such as, for example, genomic DNA or mitochondrial DNA, in the case of a DNA nucleic acid) in which it is typically found in nature, or is separated from at least some of the components of the cell in which it was produced, for example, in the case of an RNA nucleic acid. Thus, a DNA nucleic acid that is contained in a vector inside a host cell may be referred to as “isolated”.

The term “HER2” as used herein, has its general meaning in the art and refers to the Human Epidermal Growth Factor Receptor 2 (HER2) protein. HER2 is also known as Her-2, Her-2/Neu, Neu, ErbB-2, CD340 (cluster of differentiation 340) or p185, all of which terms are encompassed herein in “HER2”. The extracellular domain of HER2 comprises four domains, Domain I (amino acid residues from about 1-195), Domain II (amino acid residues from about 196-319), Domain III (amino acid residues from about 320-488), and Domain IV (amino acid residues from about 489-630) (residue numbering without signal peptide). See Garrett et al., Mol Cell. (2003) 11:495-505, Cho et al., Nature (2003) 421:756-60, Franklin et al., Cancer Cell (2004) 5:317-28, Tse et al., Cancer Treat Rev. (2012) 38(2):133-42, or Plowman et al., Proc Natl Acad Sci. (1993) 90:1746-50. In some embodiments, the HER2-binding agents provided herein bind the extracellular domain of a HER2 protein having an amino acid sequence disclosed in Genbank Reference: NP_001005862, NP_001276865, NP_001276866, NP_001276867, and/or NP_004439 (as of Jan. 1, 2020).

The terms VHH (Variable domain of the Heavy chain of the Heavy chain antibody) and “single-domain antibody” are used interchangeably herein to refer to a single antigen-binding antibody fragment, composed of a polypeptide sequence having three complementarity-determining regions (CDRs). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively. A VHH typically has a molecular weight of 12-15 kDa. The VHH binding domain(s) of the HER2-binding agents provided herein are capable of binding to HER2 alone without pairing with a corresponding CDR-containing polypeptide. The term “VHH binding domain” is used herein to refer to a full length VHH, a VHH binding fragment and/or a VHH variant that binds an antigen of interest, and/or fragments or variants of a VHH that binds to an antigen of interest (e.g., HER2), unless the context dictates otherwise. The provided HER2-binding VHH domains can be engineered from any species including, but not limited to, mouse, human, camel, llama, alpaca, vicuna, guanaco, shark, goat, rabbit, and/or bovine. In some embodiments, the VHH binding domain is derived from a Camelidae species (e.g., a camel, a llama, a dromedary, an alpaca, a vicuna and a guanaco).

The terms “HER2-binding VHH,” “HER2-binding VHH domain,” and “VHH that specifically binds HER2” are used interchangeably herein to refer to a HER2-binding VHH or HER2-binding VHH domain, unless the context dictates otherwise.

“Humanized” forms of a VHH binding domain are binding domains that contain minimal sequence derived from non-human immunoglobulin or non-human germline sequence. In some embodiments, a humanized antibody is a human immunoglobulin (recipient antibody) in which residues from a CDR (hereinafter defined) of the recipient are replaced by residues from a CDR of a non-human species (donor antibody) such as mouse, rat, rabbit, camel, llama, alpaca, or non-human primate having the desired specificity, affinity, and/or capacity. In some instances, framework (“FR”) residues of the human immunoglobulin are replaced by corresponding non-human residues. Furthermore, humanized antibodies can comprise residues that are not found in the recipient antibody or in the donor antibody. These modifications can be made to further refine antibody performance, such as binding affinity, isomerization, immunogenicity, radiolabeling efficiency, catabolism, enzymatic digestion, etc. The number of these amino acid substitutions in the FR is typically no more than 6. As will be appreciated, a humanized sequence can be identified by its primary sequence and does not necessarily denote the process by which the VHH binding domain was created. Methods for humanizing a VHH binding domain are well known in the art. Typically, the humanizing substitutions are chosen such that the resulting humanized VHH domain still retains the favorable properties of the HER2-binding agents of the disclosure.

The terms “specifically binds,” “specifically recognizes,” and “is specific for” are used interchangeably herein to refer to measurable and reproducible interactions such as binding between a target and an antigen binding protein (such as HER2 and a HER2-binding agent provided herein), which is determinative of the presence of the target in the presence of a heterogeneous population of molecules including the target. For example, an antigen binding protein that specifically binds a target (which can be an epitope) is an antigen binding protein that binds this target with greater affinity, avidity, more readily, and/or with greater duration than it binds other targets. In some embodiments, the extent of binding of an antigen binding protein to an unrelated target is less than about 10% of the binding of the antigen binding protein (e.g., HER2-binding agent) to the target (e.g., HER2) as measured, e.g., by a radioimmunoassay (RIA). In some embodiments, an antibody is said to specifically bind to an antigen when the K_{D} for the binding is ≤1 μM, e.g., ≤100 nM or ≤10 nM. The terms “HER2-binding polypeptide(s)”, “HER2-binding protein(s)” and “HER2-binding VHH domain(s)” and thereof refer to polypeptides and VHH domains that specifically bind HER2, respectively.

“Affinity” refers to the strength of the sum total of noncovalent interactions between, the binding interface of a ligand (e.g. a HER2-binding molecule or agent) and the binding site on its partner molecule (e.g., the HER2 receptor). The affinity or the apparent affinity of a molecule X for its partner Y can generally be represented by the dissociation constant (K_{D}) or the K_{D}-apparent, respectively. The affinity can also be represented by the ratio of its kinetic binding on-rate (kₒₙ) and its kinetic release off-rate (k_{off}) (K_{D}=k_{off}/kₒₙ). Affinity can be measured by common methods known in the art (such as, for example, ELISA K_{D}, KinExA, flow cytometry, and/or surface plasmon resonance devices), including those described herein. Such methods include, but are not limited to, methods involving BIACAORE®, Octet®, or flow cytometry. The term “K_{D}”, as used herein, refers to the equilibrium dissociation constant of an antigen-binding molecule/antigen interaction. When the term “K_{D}” is used herein, it includes K_{D} and K_{D}-apparent. In some embodiments, the K_{D} of the antigen-binding molecule is measured by flow cytometry using an antigen-expressing cell line (e.g., a HER2 expressing cell) and fitting the mean fluorescence measured at each antibody concentration to a non-linear one-site binding equation (GraphPad Prism Software). In some such embodiments, the K_{D} is K_{D}-apparent.

An “affinity matured” VHH-HER2-binding domain refers to a VHH binding domain containing an amino acid sequence with one or more alterations in one or more CDRs and/or one or more FRs compared to a reference parent VHH binding domain sequence that does not possess such alterations, such alterations resulting in an improvement in the affinity of the VHH-containing polypeptide for HER2. HER2-binding agent variants include affinity matured variants of the HER2-binding VHH domains provided herein.

As used herein, a “variant” refers to a biologically active polypeptide such as a HER2-binding VHH domain that has one or more amino acid additions, substitutions, insertions and/or deletions compared to the corresponding sequence of a HER2-binding VHH domain disclosed herein. In some embodiments, a variant is a biologically active polypeptide such as a HER2-binding VHH domain having at least about 80% amino acid sequence identity with a reference amino acid sequence after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of a HER2-binding domain provided herein. In some embodiments, a variant contains an amino acid sequence that has at least about 80%, 90%, 95% or 97% amino acid sequence identity with a reference VHH binding domain. In some embodiments, a variant contains an amino acid sequence that has at least 80%, 85%, 90%, 95% 96%, 97%, 98% or 99% amino acid sequence identity with a VHH binding domain disclosed in Table 2. In some embodiments, a variant contains an amino acid sequence that has at least 80%, 85%, 90%, 95% 96%, 97%, 98% or 99% amino acid sequence identity with an amino acid sequence disclosed in any one of SEQ ID NOs: 1-81 and 205. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Exemplary computer programs to determine identity between two sequences include the GCG program package (Devereux et al., Nucleic Acids Research (1984) 12:387), BLAST, BLASTP (Karlin and Altschul, (1990) Proc Natl Acad Sci. USA 87(6):2264-8), BLASTN, and FASTA (Atschul et al., J Molec Biol. (1990) 215:403), using the default parameters of corresponding program. Certain embodiments that encompass HER2-binding variants are provided herein.

The term “vector” is used to describe a nucleic acid that can be or has been engineered to contain a cloned polynucleotide sequence that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, 0-galactosidase). The terms “expression vector,” “expression construct” or “expression cassette” are used interchangeably throughout this disclosure and are meant to include any type of genetic construct containing a nucleic acid coding for a gene product in which part or all of the nucleic acid encoding sequence is capable of being transcribed in a host cell.

A “host cell” refers to a cell that can be or has been a recipient of a vector and/or heterologous nucleic acid. Host cells can be prokaryotic cells or eukaryotic cells. Exemplary eukaryotic host cells include mammalian cells, such as primate or non-primate animal cells; fungal cells, such as yeast; plant cells; and insect cells. Non-limiting mammalian host cells encompassed by the disclosure include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes a cell transfected in vivo with a nucleic acid encoding a VHH binding domain provided herein.

As used herein, a “chimeric antigen receptor” or “CAR” refers to an engineered receptor that introduces an antigen specificity, via an antigen binding domain (e.g., a HER2-binding VHH domain disclosed herein), onto cells to which it is engineered (for example T cells such as naïve T cells, central memory T cells, effector memory T cells or any combination thereof). This combines the antigen binding properties of the antigen binding domain with the T cell activity (e.g., lytic capacity and self-renewal) of T cells. A CAR typically includes an extracellular antigen-binding domain (ectodomain), a transmembrane domain and an intracellular signaling domain. The intracellular signaling domain generally contains at least one ITAM signaling domain, e.g., derived from CD3 zeta, and optionally at least one costimulatory signaling domain, e.g., derived from CD28 or 4-1BB. In a CAR provided herein, a HER2-binding VHH domain provided herein forms the antigen binding domain and is located at the extracellular side when expressed in a cell.

The term “biological activity” refers to any one or more biological properties of a molecule (whether present naturally as found in vivo, or provided or enabled by recombinant means). Biological properties include, but are not limited to, binding a ligand (e.g., HER2), inducing or increasing cell death, inducing or increasing cell proliferation, inducing or increasing cellular internalization, inducing or increasing intracellular retention, and inducing or increasing expression of cytokines.

“Pharmaceutically acceptable” refers to molecular entities and compositions that do not produce an adverse, allergic or other untoward reaction when administered to a mammal, especially a human, as appropriate. A pharmaceutically acceptable carrier refers to a non-toxic solid, semi-solid or liquid filler, diluent, encapsulating material or formulation auxiliary of any type. Pharmaceutically acceptable carriers include, but are not limited to, buffers, carriers, excipients, stabilizers, diluents, preservatives, solvents, dispersion media, coatings, antibacterial and antifungal agents, isotonic and absorption delaying agents, and the like, compatible with pharmaceutical administration. Suitable examples of such carriers or diluents include, but are not limited to, water, saline, Ringer's solutions, dextrose solution, and 5% human serum albumin. Suitable carriers are described in the most recent edition of Remington's Pharmaceutical Sciences, a standard reference text in the field, which is incorporated herein by reference. Supplementary biologically active agents (e.g., therapeutic agents) can also be incorporated into the compositions.

The term “radionuclide” refers to a moiety comprising a radioactive isotope of at least one element. Examples of radionuclides that can be used according to the present disclosure include ¹⁸F, ³⁵S, ⁷⁵Br, ⁷⁶Br, ⁷⁷Br, ⁸⁹Zr, ^{99m}Tc, ⁶⁷Ga, ⁶⁸Ga, ⁶⁴Cu, ⁶⁷Cu, ⁵²Mn, ⁹⁰Y, ¹¹In, ¹⁷⁷Lu, ¹⁵³Sm, ¹⁶⁶Ho, ¹²²I, ¹²³I, ¹²⁴I, ¹²⁵I, ¹³¹I, ²¹¹At, ²¹²Pb, ²¹²Bi, ²¹³Bi, ²²⁷Th, and ²²⁵Ac. In some embodiments the radionuclide is an alpha radionuclide (e.g., ²¹¹At or ²²⁵Ac). In some embodiments the radionuclide is a beta radionuclide (e.g., ¹³¹I or ¹⁷⁷Lu). In some embodiments the radionuclide is a radioactive halogen isotope or a radioactive metal isotope. In some embodiments, the radionuclide is a radioactive halogen isotope. In some embodiments, the radionuclide is a radioactive halogen isotope selected from: ¹⁸F, ⁷⁶Br, ¹²³I, ¹²⁴I, ¹²⁵I, and ¹³¹I or ⁷⁵Br, ⁷⁷Br, ¹²²I, ¹²⁴I, ¹²⁵I, ¹³¹I, and ²¹¹At. In particular embodiments, the radionuclide is ¹³¹I, ²¹¹At, ¹²⁵I, or ¹²⁴I. In some embodiments, the radionuclide is a radioactive metal isotope. In some embodiments, the radionuclide is a radioactive metal isotope selected from: ¹⁴⁴Sc, ⁴⁵Ti, ⁵¹Cr, ⁶²Cu ⁶⁴Cu, ⁶⁶Ga, ⁶⁸Ga, ⁶⁸Ge, ⁷⁵Se, ⁸²Sr, ⁸⁶Y, ⁹⁹Mo, ^{99m}Tc, ^{110m}In, ¹¹¹In, ¹⁶⁶Ho, ¹⁸⁶Re, ^{195m}Pt, and ²⁰¹Tl; or ⁴⁷Sc, ⁵²Mn, ⁶⁴Cu, ⁶⁷Cu, ⁶⁷Ga, ⁸⁹Zr, ⁹⁰Y, ¹¹¹In, ¹⁵³Sm, ¹⁴⁹Tb, ¹⁶¹Tb, ¹⁶⁶Ho, ¹⁷⁷Lu, ¹⁸⁸Re, ²¹²Pb, ²¹²Bi, ²¹³Bi, ²²⁵Ac, ²²⁶Th, and ²²⁷Th. In particular embodiments, the radionuclide is ¹⁷⁷Lu, ¹³¹I, ²¹¹At, or ²²⁵Ac. In some embodiments, the radionuclide is useful in therapy. In certain embodiments, the radionuclide is selected from: ⁷⁵Br, ⁷⁷Br, ¹²²I, ¹²⁴I, ¹²⁵I, ¹³¹I, and ²¹¹At; ⁴⁷Sc, ⁵²Mn, ⁶⁴Cu, ⁶⁷Cu, ⁶⁷Ga ⁸⁹Zr, ⁹⁰Y ¹¹¹In, ¹⁵³Sm, ¹⁴⁹Tb, ¹⁶¹Tb, ¹⁶⁶Ho, ¹⁷⁷Lu, ¹⁸⁸Re, ²¹²Pb, ²¹²Bi, ²¹³Bi, ²²⁵Ac, ²²⁶Th, and ²²⁷Th; or ³²P. In some embodiments, the radionuclide is useful in diagnostics. In certain embodiments, the radionuclide is selected from: ¹⁸F, ⁷⁶Br, ¹²³I, ¹²⁴I, ¹²⁵I, and ¹³¹I; ⁴⁴Sc, ⁴⁵Ti, ⁵¹Cr, ⁶²Cu, ⁶⁴Cu, ⁶⁶Ga, ⁶⁸Ga, ⁶⁸Ge, ⁷⁵Se, ⁸²Sr, ⁸⁶Y, ⁹⁹Mo, ^{99m}Tc, ^{110m}In, ¹¹¹In, ¹⁶⁶Ho, ¹⁸⁶Re, ^{195m}Pt, and ²⁰¹Tl; or ¹¹C, ¹³N, ¹⁵O, ²⁴Na, ³²P, and ¹³³Xe.

The term “prosthetic group” or “bifunctional labeling agent” refers to a small organic molecule that can be chemically linked to contain a radionuclide that is capable of being linked to a peptide or protein (e.g., a protein containing a HER2-binding VHH domain). Radiolabeled prosthetic agents are compounds or radicals that generally include a radiolabel, a charged group (CG), and a macromolecule conjugating moiety (MMCM), suitable for attachment to a protein component of a HER2-binding agent. Each of these components can optionally be associated with one or more cleavable (or non-cleavable) linkers.

The terms “chelator ligand” or “chelating agent” as used herein with respect to radiopharmaceutical chemistry refers to a bifunctional chelator or conjugating (BFC) moiety that covalently links a radiolabeled prosthetic group to a biologically active targeting molecule (e.g., a peptide or protein such as a protein comprising a HER2-binding VHH domain disclosed herein). BFCs utilize functional groups such as carboxylic acids or activated esters for amide couplings, isothiocyanates for thiourea couplings and maleimides for thiol couplings.

The terms “label” and “detectable label” refer to a polypeptide or moiety attached, for example, to a protein comprising a HER2-binding VHH domain that renders detectable a reaction (for example, binding) between the members of a specific binding pair. Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a protein of biotinyl moieties that can be detected by marked avidin (for example, streptavidin containing a fluorescent marker or enzymatic activity that can be detected by optical or colorimetric methods). Examples of labels for proteins include, but are not limited to: radioisotopes or radionuclides (for example, ¹⁸F, ³⁵S, ⁷⁵Br, ⁷⁶Br, ⁷⁷Br, ⁸⁹Zr, ^{99m}Tc, ⁶⁷Ga, ⁶⁸Ga, ⁶⁴Cu, ⁶⁷Cu, ⁵²Mn, ⁹⁰Y, ¹¹¹In, ¹⁷⁷Lu, ¹⁵³Sm, ¹⁶⁶Ho, ¹²²I, ¹²³I, ¹²⁴I, ¹²⁵I, ¹³¹I, ²¹¹At, ²¹²Pb, ²¹²Bi, ²¹³Bi, ²²⁷Th, and ²²⁵Ac); chromogens, fluorescent dyes (for example, FITC, rhodamine, lanthanide phosphors), enzymatic labels (for example, horseradish peroxidase, luciferase, alkaline phosphatase); chemiluminescent markers; biotinyl groups; predetermined protein epitopes recognized by a secondary reporter (for example, leucine zipper pair sequences, binding sites for secondary antibodies, metal binding domains, epitope tags); toxins, chemotherapeutic agents, and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

The terms “subject,” “patient,” “animal,” and “individual,” are used interchangeably and refer to mammals such as human patients and non-human primates, as well as experimental animals such as rabbits, rats, and mice, and other animals. Animals include all vertebrates, e.g., mammals and non-mammals, such as chickens, amphibians, and reptiles. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and other members of the class Mammalia. The patient can be male or female and can be any suitable age, including infant, juvenile, adolescent, adult, and geriatric subjects. In some examples, an “individual” or “subject” refers to an individual or subject in need of treatment for a disease or disorder such as cancer. In particular embodiments, the disease or disorder is HER2⁺ cancer. In certain embodiments, the disease or disorder is HER2⁺ breast cancer, or a breast cancer brain metastasis. In certain embodiments, the disease or disorder is HER2⁺ ovarian cancer, cervical cancer, endometrial cancer, head and neck cancer, brain cancer, gastric cancer (e.g., colorectal and colon cancer), pancreatic cancer, renal cancer, prostate cancer, lung cancer (e.g., NSCLC), bladder cancer, urothelial cancer, an epidermal-derived cancer, and/or a metastasis originating therefrom (e.g., an ovarian cancer brain metastasis).

In some embodiments, the subject to receive the treatment can be a patient, designating the fact that the subject has been identified as having a disorder of relevance to the treatment, or being at adequate risk of contracting the disorder. In particular embodiments, the patient is a human.

The term “biological sample” refers to a quantity of a substance from a living thing such as a mammal, or a formerly living thing. Such substances include, but are not limited to, blood (for example, whole blood), plasma, serum, urine, amniotic fluid, synovial fluid, endothelial cells, other cells, organs, tissues, bone marrow, lymph nodes and spleen. In particular embodiments, the biological sample is derived from a tissue biopsy (e.g., a HER2⁺ tissue and/or a cancerous tissue such as breast cancer, ovarian cancer, cervical cancer, endometrial cancer, head and neck cancer, brain cancer, gastric cancer (e.g., colorectal and colon cancer), pancreatic cancer, renal cancer, prostate cancer, lung cancer (e.g., NSCLC), bladder cancer, urothelial cancer, an epidermal-derived cancer, and/or a metastasis originating therefrom (e.g., a breast cancer brain metastasis)).

As used herein, the terms “treating,” “treatment,” or “therapy” of a disease, disorder or condition is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, such as metastasis to the brain, lung, or lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In line with the above, the term “treatment” does not require one-hundred percent removal of all aspects of the disorder.

The terms “treatment of a proliferative disorder,” “treatment of a hyperproliferative disorder” and iterations thereof, are used herein to include maintaining or decreasing tumor size, inducing tumor regression (either partial or complete), inhibiting tumor growth, and/or increasing the life span of a subject having the proliferative disorder. In some embodiments, the proliferative disorder is a HER2⁺ solid tumor. Such tumors include HER2⁺ cancers. In some embodiments, the tumor is associated with breast cancer, ovarian cancer, cervical cancer, endometrial cancer, head and neck cancer, brain cancer, gastric cancer (e.g., colorectal and colon cancer), pancreatic cancer, renal cancer, prostate cancer, lung cancer (e.g., NSCLC), bladder cancer, urothelial cancer, an epidermal-derived cancer, and/or metastase(s) originating therefrom (e.g., breast cancer that has metastasized to the brain).

The terms “tumor cell,” “cancer cell,” “cancer,” “tumor,” and/or “neoplasm,” unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases. The types of cancers that can be treated with the methods provided herein include solid tumors, disseminated metastatic disease, and brain metastases.

The term “anti-cancer agent” is used herein in its broadest sense to refer to agents that are used in the treatment of one or more cancers. Exemplary classes of anti-cancer agents include, but are not limited to, chemotherapeutic agents, anti-cancer biologics (such as cytokines, receptor extracellular domain-Fc fusions, and antibodies), radiation therapy, CAR-T therapy, therapeutic oligonucleotides (such as antisense oligonucleotides and siRNAs) and oncolytic viruses.

The terms “effective amount” or “therapeutically effective amount” refer to a quantity and/or concentration of a composition containing an active ingredient (e.g., a HER2-binding compound) that when administered into a patient either alone (i.e., as a monotherapy) or in combination with an additional therapeutic agent, yields a statistically significant decrease in disease progression, for example, by ameliorating or eliminating symptoms and/or the cause of the disease. An effective amount may be an amount that relieves, lessens, or alleviates at least one symptom or biological response or effect associated with a disease or disorder, prevents progression of the disease or disorder, or improves physical functioning of the patient. In reference to cancer, an effective amount of a drug or composition is an amount sufficient to: (i) reduce the number of cancer cells; (ii) reduce tumor size; (iii) inhibit, retard, slow to some extent and in some cases stop cancer cell infiltration into peripheral organs; (iv) inhibit (i.e., slow to some extent and in some cases stop) tumor metastasis; (v) inhibit tumor growth; (vi) prevent or delay occurrence and/or recurrence of tumor; and/or (vii) relieve to some extent one or more of the symptoms associated with the cancer. In some embodiments, an effective amount is an amount sufficient to prevent or delay development. In some embodiments, an effective amount is an amount sufficient to prevent or delay recurrence. An effective amount can be administered in one or more administrations. The therapeutically effective amount of a composition containing an active agent may vary according to factors such as the disease state, age, sex, and weight of the individual, and the ability of the active agent to elicit a desired response in the individual. A therapeutically effective amount is also one in which any toxic or detrimental effects of the active agent are outweighed by the therapeutically beneficial effects. A therapeutically effective amount can be administered in one or more administrations.

The disclosed HER2-binding agents and pharmaceutical compositions containing these agents can be administered alone (i.e., monotherapy) or in combination with an additional therapeutic agent (i.e., combination therapy). Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effects of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

An “article of manufacture” is any article (for example, a package or container) or kit comprising at least one reagent, for example, a medicament described herein for treatment of a disease or disorder described herein (for example, cancer), or a probe for specifically detecting a biomarker described herein. In some embodiments, the article or kit is promoted, distributed, or sold as a unit for performing the methods described herein.

The term “package insert” is used to refer to instructions customarily included in commercial packages of therapeutic products, that contain information about the indications, usage, dosage, administration, combination therapy, contraindications and/or warnings concerning the use of such therapeutic products.

### I. HER2-Binding Agent

The disclosure provides HER2-binding agents that contain a protein comprising a HER2-binding VHH domain.

In some embodiments, the provided HER2-binding agent binds HER2 and has a dissociation constant (K^{d}) of ≤10⁻⁷ M, ≤10⁻⁸ M, ≤10⁻⁹ M, ≤10⁻¹⁰ M, or ≤10⁻¹¹ M. In some embodiments, the HER2-binding agent binds HER2 and has a K_{d} of 10⁻⁷ to 10⁻¹¹ M or 10⁻⁸ to 10⁻¹⁰ M.

In some embodiments, the provided HER2-binding agent binds HER2 with a K_{D} of about 500 nm or less or 1 nm or less. In some embodiments, the HER2-binding agent binds HER2 with a k_{off} of about 0.002/s or less. In some embodiments, the HER2-binding agent binds HER2 with a K_{D} of about 500 nm or less and a k_{off} of about 0.002/s or less.

In some embodiments, the HER2-binding agent contains a VHH binding domain that specifically binds an epitope on the human protein that is conserved among the HER2 proteins from different species. In some embodiments, the HER2-binding agent contains a VHH binding domain that specifically binds to HER2 Domain I. In some embodiments, the HER2-binding agent contains a VHH binding domain that specifically binds to HER2 Domain II. In certain embodiments, the VHH binding domain competes with trastuzumab for binding HER2. In some embodiments, the HER2-binding agent contains a VHH binding domain that specifically binds to HER2 Domain III. In some embodiments, the HER2-binding agent contains a VHH binding domain that specifically binds to HER2 Domain IV. In certain embodiments, VHH binding domain competes with pertuzumab for binding HER2. In some embodiments, the HER2-binding agent contains two or more HER2-binding domains that specifically bind different epitopes of HER2. In some embodiments, the HER2-binding agent contains a VHH binding domain that specifically binds one HER2 domain and a VHH binding domain that specifically binds to a different HER2 domain. In certain embodiments, the HER2-binding agent contains a VHH binding domain that specifically binds HER2 Domain II and a VHH binding domain that specifically binds HER2 domain IV. In some embodiments, specific binding can include, but does not require, exclusive binding to HER2.

In some embodiments, the HER2-binding agent comprises an affinity matured HER2-binding VHH domain. In some embodiments, the HER2-binding agent contains a humanized VHH binding domain. In some embodiments, the HER2-binding agent contains a protein composed of a single polypeptide. In other embodiments, the HER2-binding agent contains more than one polypeptide.

In some embodiments, the HER2-binding agent comprises a VHH amino acid sequence disclosed in Table 2. In some embodiments, the HER2-binding agent comprises a VHH amino acid sequence selected from SEQ ID NOs: 1-81 and 205.

In some embodiments, the HER2-binding agent comprises a HER2-binding fragment and/or variant of a HER2-binding amino acid sequence provided herein. In some embodiments, the HER2-binding agent containing the fragment or variant amino acid sequence binds HER2 with a K_{D} of about 500 nm or less or about 1 nm or less. In some embodiments, the HER2-binding agent binds HER2 with a k_{off} of about 0.002/s or less. In certain embodiments, the HER2-binding agent binds HER2 with a K_{D} of about 500 nm or less or about 1 nm or less and a k_{off} of about 0.002/s or less.

In some embodiments, the HER2-binding agent comprises an amino acid sequence having at least 80%, 85%, 90%, 95% 96%, 97%, 98% or 99% sequence identity with a HER2-binding VHH domain provided herein. Also included is a sequence which has at least 50%, 80%, 85%, 90%, 95% 96%, 97%, 98% or 99% identity, using the default parameters of the BLAST computer program provided by HGMP, thereto. In other embodiments, amino acid sequence identity is determined using a suitable sequence alignment algorithm and default parameters, such as BLAST P (Karlin and Altschul, PNAS USA (1990) 87(6):2264-8).

In some embodiments, the HER2-binding agent comprises a VHH sequence disclosed in Table 2. In some embodiments, the HER2-binding agent comprises an amino acid sequence having at least 80%, 85%, 90%, 95% 96%, 97%, 98% or 99% sequence identity with a VHH sequence disclosed in Table 2. In some embodiments, the HER2-binding agent comprises an amino acid sequence selected from SEQ ID NOs: 1-81 and 205. In some embodiments, the HER2-binding agent comprises an amino acid sequence having at least 80%, 85%, 90%, 95% 96%, 97%, 98% or 99% sequence identity with an amino acid sequence selected from SEQ ID NOs: 1-81 and 205.

In some embodiments, the HER2-binding agent contains a variant protein that comprises an amino acid sequence having one or more additions, insertions, substitutions and/or deletions compared to a VHH binding domain disclosed herein.

Amino acid substitutions include the replacement of one or more amino acid in a protein with one or more other amino acids, respectively. Exemplary substitutions are shown in Table 1. In some embodiments, a conservative substitution in a provided HER2-binding agent variant entails exchanging an original residue in a VHH sequence disclosed in Table 2 with an amino acid residue in the corresponding exemplary substitution list in Table 1.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys, Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys, Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val, Ile: Ala, Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Additionally, amino acids may be grouped according to common side-chain properties: (1) hydrophobic: norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. In some embodiments, conservative substitutions entail exchanging two members within one of these groups. In some embodiments, non-conservative substitutions entail exchanging a member of one of these groups with a member from another group.

In some embodiments, the HER2-binding agent comprises one or more substitutions selected from: a Lys residue with either an Arg or His residue; an unpaired Cys residue with an Ala or Ser residue; and a Glu residue with an Asn residue.

In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) amino acid residue additions, substitutions and/or deletions compared to a reference HER2-binding VHH domain provided herein. Amino acid deletions or insertions may also be made relative to the amino acid sequence of a HER2-binding VHH provided herein. Thus, for example, amino acids which do not have a substantial effect on the activity of the HER2-binding domain, or at least which do not eliminate such activity, may be deleted. Such deletions can be advantageous since the overall length and the molecular weight of a polypeptide can be reduced whilst still retaining activity. This can enable the amount of protein required for a particular purpose to be reduced—for example, dosage levels can be reduced. In some embodiments, one or more amino acid residues are deleted from the amino terminus of the HER2-binding VHH domain. In some embodiments, one or more amino acid residues are deleted from the carboxy terminus of the HER2-binding VHH domain. In some embodiments, one or more amino acid residues are deleted from both the amino terminus and carboxy terminus of the HER2-binding VHH domain. Amino acid insertions relative to the sequence of a HER2-binding VHH domain disclosed herein are also provided. In some embodiments, one or more additional amino acid residues are at the amino terminus of the HER2-binding VHH domain. In some embodiments, one or more additional amino acid residues are at the carboxy terminus of the HER2-binding VHH domain. In some embodiments, one or more additional amino acid residues are at the amino terminus and the carboxy terminus of the HER2-binding VHH domain. Amino acid changes relative to the sequence of a HER2-binding VHH provided herein can be made using any suitable technique known in the art, such as by using site-directed mutagenesis.

In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) amino acid residue additions, substitutions and/or deletions compared to a reference VHH sequence disclosed in Table 2. In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) additional amino acid residues compared to a reference VHH sequence disclosed in Table 2. In some embodiments, the additional amino acid residues are at the amino terminus of the HER2-binding VHH domain compared to a reference VHH sequence disclosed in Table 2. In some embodiments, the additional amino acid residues are at the carboxy terminus of the HER2-binding VHH domain compared to a reference VHH sequence disclosed in Table 2. In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) additional amino acid residues at the amino terminus of the HER2-binding VHH domain and 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) additional amino acid residues at the carboxy terminus of the HER2-binding VHH domain, compared to a reference VHH sequence disclosed in Table 2.

In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) amino acid substitutions compared to a reference VHH sequence disclosed in Table 2. In some embodiments, the amino acid substitutions are conservative. In some embodiments, the amino acid substitutions are nonconservative. A mix of conservative and nonconservative substitutions is also contemplated.

In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) amino acid residue deletions compared to a reference VHH sequence disclosed in Table 2. In some embodiments, the amino acid residues are deleted at the amino terminus of the HER2-binding VHH domain compared to a reference VHH sequence disclosed in Table 2. In some embodiments, the amino acid residues are deleted at the carboxy terminus of the HER2-binding VHH domain compared to a reference VHH sequence disclosed in Table 2. In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) amino acid residues deleted at the amino terminus of the HER2-binding VHH domain and 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) amino acid residues deleted at the carboxy terminus of the HER2-binding VHH domain, compared to a reference VHH sequence disclosed in Table 2.

In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) additions, substitutions and/or deletions compared to a reference sequence selected from SEQ ID NOs: 1-81 and 205. In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) additions compared to a reference sequence selected from SEQ ID NOs: 1-81 and 205. In some embodiments, the additional amino acid residues are at the amino terminus of the HER2-binding VHH domain compared to a sequence selected from SEQ ID NOs: 1-81 and 205. In some embodiments, the additional amino acid residues are at the carboxy terminus of the HER2-binding VHH domain compared to a sequence selected from SEQ ID NOs: 1-81 and 205. In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) additional amino acid residues at the amino terminus and 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) additional amino acid residues at the carboxy terminus, compared to a sequence selected from SEQ ID NOs: 1-81 and 205.

In some embodiments, the HER2-binding agent comprises an amino acid sequence that has 1-5, 1-3, or 1, 2, 3, 4, or 5 additions, substitutions and/or deletions compared to a reference sequence selected from SEQ ID NOs: 1-81 and 205 wherein the addition(s), substitution(s) and/or deletion(s) is/are within and/or proximal to (within 5 AA of) a VHH CDR as disclosed in Table 2. In particular embodiments, the addition(s), substitution(s) and/or deletion(s) is/are at a residue position within and/or proximal to (within 5 AA of) a VHH CDR and which may otherwise provide a potential radiolabeling site. Such addition(s), substitution(s) and/or deletion(s) is/are advantageous to Targeted Radionuclide Therapy (TRT) applications for several reasons including, for example, that it ensures that the VHH maintains its affinity, specificity, and internalization properties after being radiolabeled with its therapeutic payload; it allows the VHH to be pre-conjugated with the radiolabeling prosthetic moiety—such an approach maximizes the radiochemical labeling yield, which is of vital importance for poorly reactive or short half-live based radionuclides; it allows for site selective radiolabeling (see e.g., SEQ ID NO: 34, SEQ ID NO: 36, SEQ ID NO: 38, SEQ ID NO: 39, SEQ ID NO: 42, etc.); and it allows for better control over the consistency and heterogeneity of the VHH-drug conjugate or VHH radiolabeled conjugate.

In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) amino acid substitutions compared to a reference sequence selected from SEQ ID NOs: 1-81 and 205. In some embodiments, the amino acid substitutions are conservative. In some embodiments, the amino acid substitutions are nonconservative. A mix of conservative and nonconservative substitutions is also contemplated.

In some embodiments, the HER2-binding agent comprises an amino acid sequence that has 1-5, 1-3, or 1, 2, 3, 4, or 5 substitutions compared to a reference sequence selected from SEQ ID NOs: 1-81 and 205 wherein the substitution(s) is/are within and/or proximal to (within 5 AA) a VHH CDR as disclosed in Table 2. In particular embodiments, the substitution(s) is/are at a residue position within and/or proximal to (within 5 AA) a VHH CDR and which may otherwise provide a potential radiolabeling site.

In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) deletions compared to a reference sequence selected from SEQ ID NOs: 1-81 and 205. In some embodiments, the amino acid residues are deleted at the amino terminus of the HER2-binding VHH domain compared to a reference sequence selected from SEQ ID NOs: 1-81 and 205. In some embodiments, the amino acid residues are deleted at the carboxy terminus of the HER2-binding VHH domain compared to a reference sequence selected from SEQ ID NOs: 1-81 and 205. In some embodiments, the HER2-binding agent comprises an amino acid sequence that has 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) amino acid residues deleted at the amino terminus of the HER2-binding VHH domain and 1-25, 1-20, 1-15, 1-10, 1-5 or 1-3 (e.g., 1, 2, 3, 4, 5, 6, 7, 8, 9, 10, 11, 12, 13, 14, 15, 16, 17, 18, 19, 20, 21, 22, 23, 24, or 25) amino acid residues deleted at the carboxy terminus of the HER2-binding VHH domain, compared to a reference sequence selected from SEQ ID NOs: 1-81 and 205.

In some embodiments, the HER2-binding polypeptide comprises a fragment of the VHH sequence disclosed in any one of SEQ ID NOs: 1-81 and 205. In some embodiments, the HER2-binding agent comprises a HER2-binding fragment of a VHH amino acid sequence that contains 50-100, 50-175 contiguous amino acids of the sequence disclosed in any one of SEQ ID NOs: 1-81 and 205. In some embodiments, the HER2-binding polypeptide comprises a fragment of a sequence disclosed in Table 2. In some embodiments, the HER2-binding agent comprises a HER2-binding fragment of a VHH amino acid sequence that contains 50-100, 50-175 contiguous amino acids of the sequence disclosed in Table 2.

In some embodiments, the HER2-binding polypeptide comprises an amino acid sequence disclosed in Table 2. In some embodiments, the HER2-binding polypeptide comprises an amino acid sequence selected from SEQ ID NOs: 1-81 and 205. In some embodiments, the HER2-binding polypeptide comprises an amino acid sequence that has at least 80%, 85%, 90%, 95% 96%, 97%, 98% or 99% sequence identity with a VHH sequence disclosed in Table 2. In some embodiments, the HER2-binding polypeptide comprises an amino acid sequence that has at least 80%, 85%, 90%, 95% 96%, 97%, 98% or 99% sequence identity with a VHH having the amino acid sequence of SEQ ID NOs: 1-81 and 205. In some embodiments, the HER2-binding polypeptide comprises a complementarity determining region (CDR) 3 sequence disclosed in Table 2. In some embodiments, the HER2-binding agent contains a protein that comprises an amino acid sequence selected from SEQ ID NOs: 124-157 and 206.

In some embodiments, the HER2-binding polypeptide comprises a CDR3 sequence and a CDR2 sequence disclosed in Table 2. In some embodiments, the HER2-binding polypeptide comprises a CDR3 sequence selected from SEQ ID NOs: 124-157 and 206 and a CDR2 sequence selected from SEQ ID NOs: 102-123. In some embodiments, the HER2-binding polypeptide comprises a CDR3 sequence and a CDR2 sequence from the same VHH sequence disclosed in Table 2. In some embodiments, the HER2-binding polypeptide comprises a CDR3 sequence and a CDR2 sequence from different VHH sequences disclosed in Table 2.

In some embodiments, the HER2-binding polypeptide comprises a CDR3 sequence, a CDR2 sequence, and a CDR1 sequence disclosed in Table 2. In some embodiments, the HER2-binding polypeptide comprises a CDR3 sequence selected from SEQ ID NOs: 124-157 and 206, a CDR2 sequence selected from SEQ ID NOs: 102-123, and a CDR1 sequence selected from SEQ ID NO: 82-101. In some embodiments, the HER2-binding polypeptide comprises a CDR3 sequence, a CDR2 sequence, and a CDR1 sequence from the same VHH sequence disclosed in Table 2. In some embodiments, the HER2-binding polypeptide comprises a CDR3 sequence, a CDR2 sequence, and a CDR1 sequence from different VHH sequences disclosed in Table 2.

In some embodiments, the HER2-binding agent comprises a CDR1 comprising the amino acid sequence of SEQ ID NO: 101; a CDR2 comprising the amino acid sequence of SEQ ID NO: 111; and a CDR3 comprising the amino acid sequence of SEQ ID NO: 135 or 136. In certain embodiments, the HER2-binding agent comprises an amino acid sequence selected from SEQ ID NOs: 23-42.

In some embodiments, the HER2-binding agent comprises a CDR1 comprising the amino acid sequence of SEQ ID NO: 101; a CDR2 comprising the amino acid sequence of SEQ ID NO: 111; and a CDR3 comprising the amino acid sequence of SEQ ID NO: 135. In certain embodiments, the HER2-binding agent comprises an amino acid sequence selected from SEQ ID NOs: 23, 32, and 34-38. In some embodiments, the HER2-binding agent comprises the amino acid sequence of SEQ ID NO: 23. In some embodiments, the HER2-binding agent comprises the amino acid sequence of: SEQ ID NO: 32. In some embodiments, the HER2-binding agent comprises the amino acid sequence of: SEQ ID NO: 34. In some embodiments, the HER2-binding agent comprises the amino acid sequence of: SEQ ID NO: 35. In some embodiments, the HER2-binding agent comprises the amino acid sequence of: SEQ ID NO: 36. In some embodiments, the HER2-binding agent comprises the amino acid sequence of: SEQ ID NO: 37. In some embodiments, the HER2-binding agent comprises the amino acid sequence of: SEQ ID NO: 38.

In some embodiments, the HER2-binding agent comprises a CDR1 comprising the amino acid sequence of SEQ ID NO: 101; a CDR2 comprising the amino acid sequence of SEQ ID NO: 111; and a CDR3 comprising the amino acid sequence of SEQ ID NO: 136. In certain embodiments, the HER2-binding agent comprises an amino acid sequence selected from SEQ ID NOs: 24-31, 33, and 39-42. In some embodiments, the HER2-binding agent comprises the amino acid sequence of SEQ ID NO: 24. In some embodiments, the HER2-binding agent comprises the amino acid sequence of SEQ ID NO: 25. In some embodiments, the HER2-binding agent comprises the amino acid sequence of SEQ ID NO: 26. In some embodiments, the HER2-binding agent comprises the amino acid sequence of SEQ ID NO: 27. In some embodiments, the HER2-binding agent comprises the amino acid sequence of SEQ ID NO: 28. In some embodiments, the HER2-binding agent comprises the amino acid sequence of SEQ ID NO: 29. In some embodiments, the HER2-binding agent comprises the amino acid sequence of SEQ ID NO: 30. In some embodiments, the HER2-binding agent comprises the amino acid sequence of SEQ ID NO: 31. In some embodiments, the HER2-binding agent comprises the amino acid sequence of SEQ ID NO: 33. In some embodiments, the HER2-binding agent comprises the amino acid sequence of SEQ ID NO: 39. In some embodiments, the HER2-binding agent comprises the amino acid sequence of SEQ ID NO: 40. In some embodiments, the HER2-binding agent comprises the amino acid sequence of SEQ ID NO: 41 or 42. In some embodiments, the HER2-binding agent comprises the amino acid sequence of SEQ ID NO: 41. In some embodiments, the HER2-binding agent comprises the amino acid sequence of SEQ ID NO: 42.

In some embodiments, the HER2-binding agent comprises a CDR1 comprising the amino acid sequence of SEQ ID NO: 82; a CDR2 comprising the amino acid sequence of SEQ ID NO: 102; and a CDR3 comprising the amino acid sequence of SEQ ID NO: 124, 145, 146 or 147. In certain embodiments, the HER2-binding agent comprises an amino acid sequence selected from SEQ ID NOs: 1 and 43-49.

In some embodiments, the HER2-binding agent comprises a CDR1 comprising the amino acid sequence of SEQ ID NO: 82; a CDR2 comprising the amino acid sequence of SEQ ID NO: 102; and a CDR3 comprising the amino acid sequence of SEQ ID NO: 124. In certain embodiments, the HER2-binding agent comprises the amino acid sequence of SEQ ID NO: 1 or 43.

In some embodiments, the HER2-binding agent comprises a CDR1 comprising the amino acid sequence of SEQ ID NO: 82; a CDR2 comprising the amino acid sequence of SEQ ID NO: 102; and a CDR3 comprising the amino acid sequence of SEQ ID NO: 145. In certain embodiments, the HER2-binding agent comprises the amino acid sequence of SEQ ID NO: 44 or 45. In some embodiments, the HER2-binding agent comprises the amino acid sequence of SEQ ID NO: 44. In some embodiments, the HER2-binding agent comprises the amino acid sequence of SEQ ID NO: 45. In some embodiments, the HER2-binding agent comprises a CDR1 comprising the amino acid sequence of SEQ ID NO: 82; a CDR2 comprising the amino acid sequence of SEQ ID NO: 102; and a CDR3 comprising the amino acid sequence of SEQ ID NO: 146. In certain embodiments, the HER2-binding agent comprises the amino acid sequence of SEQ ID NO: 46 or 47. In some embodiments, the HER2-binding agent comprises the amino acid sequence of SEQ ID NO: 46. In some embodiments, the HER2-binding agent comprises the amino acid sequence of SEQ ID NO: 47. In some embodiments, the HER2-binding agent comprises a CDR1 comprising the amino acid sequence of SEQ ID NO: 82; a CDR2 comprising the amino acid sequence of SEQ ID NO: 102; and a CDR3 comprising the amino acid sequence of SEQ ID NO: 147. In certain embodiments, the HER2-binding agent comprises the amino acid sequence of SEQ ID NO: 48 or 49. In some embodiments, the HER2-binding agent comprises the amino acid sequence of SEQ ID NO: 48. In some embodiments, the HER2-binding agent comprises the amino acid sequence of SEQ ID NO: 49.

In some embodiments, the HER2-binding agent comprises a CDR1 comprising the amino acid sequence of SEQ ID NO: 83; a CDR2 comprising the amino acid sequence of SEQ ID NO: 103; and a CDR3 comprising the amino acid sequence of SEQ ID NO: 125. In certain embodiments, the HER2-binding agent comprises the amino acid sequence of SEQ ID NO: 2.

In some embodiments, the HER2-binding agent comprises a CDR1 comprising the amino acid sequence of SEQ ID NO: 84; a CDR2 comprising the amino acid sequence of SEQ ID NO: 104; and a CDR3 comprising the amino acid sequence of SEQ ID NO: 126. In certain embodiments, the HER2-binding agent comprises the amino acid sequence of SEQ ID NO: 3.

In some embodiments, the HER2-binding agent comprises a CDR1 comprising the amino acid sequence of SEQ ID NO: 85; a CDR2 comprising the amino acid sequence of SEQ ID NO: 105; and a CDR3 comprising the amino acid sequence of SEQ ID NO: 127. In certain embodiments, the HER2-binding agent comprises the amino acid sequence of SEQ ID NO: 4.

In some embodiments, the HER2-binding agent comprises a CDR1 comprising the amino acid sequence of SEQ ID NO: 86; a CDR2 comprising the amino acid sequence of SEQ ID NO: 106; and a CDR3 comprising the amino acid sequence of SEQ ID NO: 128, 130, 134 or 155. In certain embodiments, the HER2-binding agent comprises an amino acid sequence of SEQ ID NO: 5, 17, 22, 79, 80, or 81. In some embodiments, the HER2-binding agent comprises a CDR1 comprising the amino acid sequence of SEQ ID NO: 86; a CDR2 comprising the amino acid sequence of SEQ ID NO: 106; and a CDR3 comprising the amino acid sequence of SEQ ID NO: 128. In certain embodiments, the HER2-binding agent comprises the amino acid sequence of SEQ ID NO: 5. In some embodiments, the HER2-binding agent comprises a CDR1 comprising the amino acid sequence of SEQ ID NO: 86; a CDR2 comprising the amino acid sequence of SEQ ID NO: 106; and a CDR3 comprising the amino acid sequence of SEQ ID NO: 130. In certain embodiments, the HER2-binding agent comprises the amino acid sequence of SEQ ID NO: 17. In some embodiments, the HER2-binding agent comprises a CDR1 comprising the amino acid sequence of SEQ ID NO: 86; a CDR2 comprising the amino acid sequence of SEQ ID NO: 106; and a CDR3 comprising the amino acid sequence of SEQ ID NO: 134. In certain embodiments, the HER2-binding agent comprises the amino acid sequence of SEQ ID NO: 22 or 79. In some embodiments, the HER2-binding agent comprises the amino acid sequence of SEQ ID NO: 22. In some embodiments, the HER2-binding agent comprises the amino acid sequence of SEQ ID NO: 79. In some embodiments, the HER2-binding agent comprises a CDR1 comprising the amino acid sequence of SEQ ID NO: 86; a CDR2 comprising the amino acid sequence of SEQ ID NO: 106; and a CDR3 comprising the amino acid sequence of SEQ ID NO: 155. In certain embodiments, the HER2-binding agent comprises the amino acid sequence of SEQ ID NO: 80 or 81. In some embodiments, the HER2-binding agent comprises the amino acid sequence of SEQ ID NO: 80. In some embodiments, the HER2-binding agent comprises the amino acid sequence of SEQ ID NO: 81.

In some embodiments, the HER2-binding agent comprises a CDR1 comprising the amino acid sequence of SEQ ID NO: 87; a CDR2 comprising the amino acid sequence of SEQ ID NO: 117; and a CDR3 comprising the amino acid sequence of SEQ ID NO: 129. In certain embodiments, the HER2-binding agent comprises the amino acid sequence of SEQ ID NO: 6.

In some embodiments, the HER2-binding agent comprises a CDR1 comprising the amino acid sequence of SEQ ID NO: 88; a CDR2 comprising the amino acid sequence of SEQ ID NO: 118; and a CDR3 comprising the amino acid sequence of SEQ ID NO: 130. In certain embodiments, the HER2-binding agent comprises the amino acid sequence of SEQ ID NO: 7.

In some embodiments, the HER2-binding agent comprises a CDR1 comprising the amino acid sequence of SEQ ID NO: 89; a CDR2 comprising the amino acid sequence of SEQ ID NO: 108 or 112; and a CDR3 comprising the amino acid sequence of SEQ ID NO: 156. In certain embodiments, the HER2-binding agent comprises an amino acid sequence selected from SEQ ID NOs: 50, 51, and 52. In some embodiments, the HER2-binding agent comprises a CDR1 comprising the amino acid sequence of SEQ ID NO: 89; a CDR2 comprising the amino acid sequence of SEQ ID NO: 108; and a CDR3 comprising the amino acid sequence of SEQ ID NO: 156. In certain embodiments, the HER2-binding agent comprises the amino acid sequence of SEQ ID NO: 51. In some embodiments, the HER2-binding agent comprises a CDR1 comprising the amino acid sequence of SEQ ID NO: 89; a CDR2 comprising the amino acid sequence of SEQ ID NO: 112; and a CDR3 comprising the amino acid sequence of SEQ ID NO: 156. In certain embodiments, the HER2-binding agent comprises the amino acid sequence of SEQ ID NO: 50 or 52. In some embodiments, the HER2-binding agent comprises the amino acid sequence of SEQ ID NO: 50. In some embodiments, the HER2-binding agent comprises the amino acid sequence of SEQ ID NO: 52.

In some embodiments, the HER2-binding agent comprises a CDR1 comprising the amino acid sequence of SEQ ID NO: 89; a CDR2 comprising the amino acid sequence of SEQ ID NO: 108 or 112; and a CDR3 comprising the amino acid sequence of SEQ ID NO: 131 or 157. In certain embodiments, the HER2-binding agent comprises an amino acid sequence selected from SEQ ID NOs: 8, 19, 70, 71, and 72.

In some embodiments, the HER2-binding agent comprises a CDR1 comprising the amino acid sequence of SEQ ID NO: 89; a CDR2 comprising the amino acid sequence of SEQ ID NO: 108 or 112; and a CDR3 comprising the amino acid sequence of SEQ ID NO: 157. In certain embodiments, the HER2-binding agent comprises an amino acid sequence selected from SEQ ID NOs: 70, 71 and 72. In some embodiments, the HER2-binding agent comprises a CDR1 comprising the amino acid sequence of SEQ ID NO: 89; a CDR2 comprising the amino acid sequence of SEQ ID NO: 108; and a CDR3 comprising the amino acid sequence of SEQ ID NO: 157. In certain embodiments, the HER2-binding agent comprises the amino acid sequence of SEQ ID NO: 71. In some embodiments, the HER2-binding agent comprises a CDR1 comprising the amino acid sequence of SEQ ID NO: 89; a CDR2 comprising the amino acid sequence of SEQ ID NO: 112; and a CDR3 comprising the amino acid sequence of SEQ ID NO: 157. In certain embodiments, the HER2-binding agent comprises the amino acid sequence of SEQ ID NO: 70 or 72. In some embodiments, the HER2-binding agent comprises the amino acid sequence of SEQ ID NO: 70. In some embodiments, the HER2-binding agent comprises the amino acid sequence of SEQ ID NO: 72.

In some embodiments, the HER2-binding agent comprises a CDR1 comprising the amino acid sequence of SEQ ID NO: 89; a CDR2 comprising the amino acid sequence of SEQ ID NO: 108 and a CDR3 comprising the amino acid sequence of SEQ ID NO: 131. In some embodiments, the HER2-binding agent comprises the amino acid sequence of SEQ ID NO: 8 or 19. In some embodiments, the HER2-binding agent comprises the amino acid sequence of SEQ ID NO: 8. In some embodiments, the HER2-binding agent comprises the amino acid sequence of SEQ ID NO: 19.

In some embodiments, the HER2-binding agent comprises a CDR1 comprising the amino acid sequence of SEQ ID NO: 90; a CDR2 comprising the amino acid sequence of SEQ ID NO: 109; and a CDR3 comprising the amino acid sequence of SEQ ID NO: 132. In certain embodiments, the HER2-binding agent comprises the amino acid sequence of SEQ ID NO: 9.

In some embodiments, the HER2-binding agent comprises a CDR1 comprising the amino acid sequence of SEQ ID NO: 91; a CDR2 comprising the amino acid sequence of SEQ ID NO: 119; and a CDR3 comprising the amino acid sequence of SEQ ID NO: 137. In certain embodiments, the HER2-binding agent comprises the amino acid sequence of SEQ ID NO: 10.

In some embodiments, the HER2-binding agent comprises a CDR1 comprising the amino acid sequence of SEQ ID NO: 92; a CDR2 comprising the amino acid sequence of SEQ ID NO: 120; and a CDR3 comprising the amino acid sequence of SEQ ID NO: 138. In certain embodiments, the HER2-binding agent comprises the amino acid sequence of SEQ ID NO: 11.

In some embodiments, the HER2-binding agent comprises a CDR1 comprising the amino acid sequence of SEQ ID NO: 93; a CDR2 comprising the amino acid sequence selected from SEQ ID NO: 113 or 114; and a CDR3 comprising the amino acid sequence of SEQ ID NO: 139 or 206. In certain embodiments, the HER2-binding agent comprises an amino acid sequence selected from SEQ ID NOs: 12, 56, 57, and 58. In some embodiments, the HER2-binding agent comprises a CDR1 comprising the amino acid sequence of SEQ ID NO: 93; a CDR2 comprising the amino acid sequence of SEQ ID NO: 113; and a CDR3 comprising the amino acid sequence of SEQ ID NO: 139. In certain embodiments, the HER2-binding agent comprises the amino acid sequence of SEQ ID NO: 56 or 58. In some embodiments, the HER2-binding agent comprises the amino acid sequence of SEQ ID NO: 56. In some embodiments, the HER2-binding agent comprises the amino acid sequence of SEQ ID NO: 58. In some embodiments, the HER2-binding agent comprises a CDR1 comprising the amino acid sequence of SEQ ID NO: 93; a CDR2 comprising the amino acid sequence of SEQ ID NO: 114; and a CDR3 comprising the amino acid sequence of SEQ ID NO: 139. In certain embodiments, the HER2-binding agent comprises the amino acid sequence of SEQ ID NO: 57. In some embodiments, the HER2-binding agent comprises a CDR1 comprising the amino acid sequence of SEQ ID NO: 93; a CDR2 comprising the amino acid sequence of SEQ ID NO: 114; and a CDR3 comprising the amino acid sequence of SEQ ID NO: 206. In some embodiments, the HER2-binding agent comprises the amino acid sequence of SEQ ID NO: 12.

In some embodiments, the HER2-binding agent comprises a CDR1 comprising the amino acid sequence of SEQ ID NO: 94; a CDR2 comprising the amino acid sequence of SEQ ID NO: 115 and a CDR3 comprising the amino acid sequence selected from SEQ ID NO: 140 or 149. In certain embodiments, the HER2-binding agent comprises an amino acid sequence selected from SEQ ID NOs: 13, 59, 60, and 61. In some embodiments, the HER2-binding agent comprises a CDR1 comprising the amino acid sequence of SEQ ID NO: 94; a CDR2 comprising the amino acid sequence of SEQ ID NO: 115 and a CDR3 comprising the amino acid sequence of SEQ ID NO: 140. In certain embodiments, the HER2-binding agent comprises the amino acid sequence of SEQ ID NO: 13 or 59. In some embodiments, the HER2-binding agent comprises the amino acid sequence of SEQ ID NO: 13. In some embodiments, the HER2-binding agent comprises the amino acid sequence of SEQ ID NO: 59. In some embodiments, the HER2-binding agent comprises a CDR1 comprising the amino acid sequence of SEQ ID NO: 94; a CDR2 comprising the amino acid sequence of SEQ ID NO: 115 and a CDR3 comprising the amino acid sequence of SEQ ID NO: 149. In certain embodiments, the HER2-binding agent comprises the amino acid sequence of SEQ ID NO: 60 or 61. In some embodiments, the HER2-binding agent comprises the amino acid sequence of SEQ ID NO: 60. In some embodiments, the HER2-binding agent comprises the amino acid sequence of SEQ ID NO: 61.

In some embodiments, the HER2-binding agent comprises a CDR1 comprising the amino acid sequence of SEQ ID NO: 95; a CDR2 comprising the amino acid sequence of SEQ ID NO: 121; and a CDR3 comprising the amino acid sequence of SEQ ID NO: 141. In certain embodiments, the HER2-binding agent comprises the amino acid sequence of SEQ ID NO: 14.

In some embodiments, the HER2-binding agent comprises a CDR1 comprising the amino acid sequence of SEQ ID NO: 96; a CDR2 comprising the amino acid sequence of SEQ ID NO: 122; and a CDR3 comprising the amino acid sequence of SEQ ID NO: 142. In certain embodiments, the HER2-binding agent comprises the amino acid sequence of SEQ ID NO: 15.

In some embodiments, the HER2-binding agent comprises a CDR1 comprising the amino acid sequence of SEQ ID NO: 97; a CDR2 comprising the amino acid sequence of SEQ ID NO: 123; and a CDR3 comprising the amino acid sequence of SEQ ID NO: 143. In certain embodiments, the HER2-binding agent comprises the amino acid sequence of SEQ ID NO: 16.

In some embodiments, the HER2-binding agent comprises a CDR1 comprising the amino acid sequence of SEQ ID NO: 97; a CDR2 comprising the amino acid sequence of SEQ ID NO: 116; and a CDR3 comprising the amino acid sequence of SEQ ID NO: 150. In certain embodiments, the HER2-binding agent comprises the amino acid sequence of SEQ ID NO: 62.

In some embodiments, the HER2-binding agent comprises a CDR1 comprising the amino acid sequence of SEQ ID NO: 98; a CDR2 comprising the amino acid sequence of SEQ ID NO: 107; and a CDR3 comprising the amino acid sequence of SEQ ID NO: 144, 151, 152 or 153. In certain embodiments, the HER2-binding agent comprises an amino acid sequence selected from SEQ ID NOs: 18 and 63-69.

In some embodiments, the HER2-binding agent comprises a CDR1 comprising the amino acid sequence of SEQ ID NO: 98; a CDR2 comprising the amino acid sequence of SEQ ID NO: 107; and a CDR3 comprising the amino acid sequence of SEQ ID NO: 144. In certain embodiments, the HER2-binding agent comprises the amino acid sequence of SEQ ID NO: 18 or 63. In some embodiments, the HER2-binding agent comprises a CDR1 comprising the amino acid sequence of SEQ ID NO: 98; a CDR2 comprising the amino acid sequence of SEQ ID NO: 107; and a CDR3 comprising the amino acid sequence of SEQ ID NO: 151. In certain embodiments, the HER2-binding agent comprises the amino acid sequence of SEQ ID NO: 64 or 65. In some embodiments, the HER2-binding agent comprises the amino acid sequence of SEQ ID NO: 64. In some embodiments, the HER2-binding agent comprises the amino acid sequence of SEQ ID NO: 65.

In some embodiments, the HER2-binding agent comprises a CDR1 comprising the amino acid sequence of SEQ ID NO: 98; a CDR2 comprising the amino acid sequence of SEQ ID NO: 107; and a CDR3 comprising the amino acid sequence of SEQ ID NO: 152 or 153. In some embodiments, the HER2-binding agent comprises a CDR1 comprising the amino acid sequence of SEQ ID NO: 98; a CDR2 comprising the amino acid sequence of SEQ ID NO: 107; and a CDR3 comprising the amino acid sequence of SEQ ID NO: 152. In certain embodiments, the HER2-binding agent comprises the amino acid sequence of SEQ ID NO: 66 or 67. In some embodiments, the HER2-binding agent comprises the amino acid sequence of SEQ ID NO: 66. In some embodiments, the HER2-binding agent comprises the amino acid sequence of SEQ ID NO: 67. In some embodiments, the HER2-binding agent comprises a CDR1 comprising the amino acid sequence of SEQ ID NO: 98; a CDR2 comprising the amino acid sequence of SEQ ID NO: 107; and a CDR3 comprising the amino acid sequence of SEQ ID NO: 153. In certain embodiments, the HER2-binding agent comprises the amino acid sequence of SEQ ID NO: 68 or 69. In some embodiments, the HER2-binding agent comprises the amino acid sequence of SEQ ID NO: 68. In some embodiments, the HER2-binding agent comprises the amino acid sequence of SEQ ID NO: 69.

In some embodiments, the HER2-binding agent comprises a CDR1 comprising the amino acid sequence of SEQ ID NO: 99; a CDR2 comprising the amino acid sequence of SEQ ID NO: 109; and a CDR3 comprising the amino acid sequence of SEQ ID NO: 132 or 148. In certain embodiments, the HER2-binding agent comprises an amino acid sequence selected from SEQ ID NOs: 20, 53, 54, 55, and 73-75. In some embodiments, the HER2-binding agent comprises a CDR1 comprising the amino acid sequence of SEQ ID NO: 99; a CDR2 comprising the amino acid sequence of SEQ ID NO: 109; and a CDR3 comprising the amino acid sequence of SEQ ID NO: 132. In certain embodiments, the HER2-binding agent comprises an amino acid sequence selected from SEQ ID NOs: 20, 53, and 73. In some embodiments, the HER2-binding agent comprises the amino acid sequence of SEQ ID NO: 20. In some embodiments, the HER2-binding agent comprises the amino acid sequence of SEQ ID NO: 53. In some embodiments, the HER2-binding agent comprises the amino acid sequence of SEQ ID NO: 73. In some embodiments, the HER2-binding agent comprises a CDR1 comprising the amino acid sequence of SEQ ID NO: 99; a CDR2 comprising the amino acid sequence of SEQ ID NO: 109; and a CDR3 comprising the amino acid sequence of SEQ ID NO: 148. In certain embodiments, the HER2-binding agent comprises an amino acid sequence selected from SEQ ID NOs: 54, 55, 74 and 75. In some embodiments, the HER2-binding agent comprises the amino acid sequence of SEQ ID NO: 54. In some embodiments, the HER2-binding agent comprises the amino acid sequence of SEQ ID NO: 55. In some embodiments, the HER2-binding agent comprises the amino acid sequence of SEQ ID NO: 74. In some embodiments, the HER2-binding agent comprises the amino acid sequence of SEQ ID NO: 75.

In some embodiments, the HER2-binding agent comprises a CDR1 comprising the amino acid sequence of SEQ ID NO: 100; a CDR2 comprising the amino acid sequence of SEQ ID NO: 110; and a CDR3 comprising the amino acid sequence of SEQ ID NO: 133 or 154. In certain embodiments, the HER2-binding agent comprises an amino acid sequence selected from SEQ ID NOs: 21, 76, 77 and 78. In some embodiments, the HER2-binding agent comprises a CDR1 comprising the amino acid sequence of SEQ ID NO: 100; a CDR2 comprising the amino acid sequence of SEQ ID NO: 110; and a CDR3 comprising the amino acid sequence of SEQ ID NO: 133. In certain embodiments, the HER2-binding agent comprises the amino acid sequence of SEQ ID NO: 21 or 76. In some embodiments, the HER2-binding agent comprises the amino acid sequence of SEQ ID NO: 21. In some embodiments, the HER2-binding agent comprises the amino acid sequence of SEQ ID NO: 76. In some embodiments, the HER2-binding agent comprises a CDR1 comprising the amino acid sequence of SEQ ID NO: 100; a CDR2 comprising the amino acid sequence of SEQ ID NO: 110; and a CDR3 comprising the amino acid sequence of SEQ ID NO: 154. In certain embodiments, the HER2-binding agent comprises the amino acid sequence of SEQ ID NO: 77 or 78. In some embodiments, the HER2-binding agent comprises the amino acid sequence of SEQ ID NO: 77. In some embodiments, the HER2-binding agent comprises the amino acid sequence of SEQ ID NO: 78.

In some embodiments, the HER2-binding agent comprises a CDR1 comprising the amino acid sequence of SEQ ID NO: 101; a CDR2 comprising the amino acid sequence of SEQ ID NO: 111; and a CDR3 comprising the amino acid sequence of SEQ ID NO: 136. In certain embodiments, the HER2-binding agent comprises the amino acid sequence of SEQ ID NO: 205.

In some embodiments, the HER2-binding agent contains a protein that comprises at least one VHH binding domain provided herein that specifically binds HER2 fused to a heterologous peptide or protein sequence. In some embodiments, the HER2-binding VHH domain is fused to additional N-terminal and/or C-terminal amino acid sequences. Such additional sequences can be provided for various reasons, for example, to increase glycosylation, to improve protein expression, solubility, to enhance tumor cell internalization, or to increase ease of purification and/or to improve labeling (e.g., radiolabeling) selectivity and loading.

In some embodiments, the HER2-binding agent contains a protein that contains an additional N-terminal amino acid sequence that improves protein expression, solubility, and/or ease of purification. In some embodiments, the HER2-binding VHH domain comprises a MetAla or an Ala at the N-terminus. In some embodiments, the HER2-binding VHH domain is fused to a heterologous peptide sequence which is a poly-amino acid sequence, for example a plurality of histidine residues or a plurality of lysine residues (suitably 2, 3, 4, 5, or 6 residues).

In some embodiments, the HER2-binding agent contains a protein that contains a C-terminal amino acid sequence that improves or enables site conjugation to radiolabeled prosthetic groups, or direct labeling (e.g., radiolabeling). In some embodiments, the protein of the HER2-binding agent further comprises the C-terminal amino acid sequence (GlyₙXaaₘGlyₖ)ⱼ, wherein: VHH is a HER2-binding VHH domain provided herein (e.g., in Table 2), n=0-5, m=0-5, k=0-6, j=0-8, and Xaa is an amino acid residue that enables site conjugation to a labeled prosthetic group (e.g., a radiolabeled prosthetic group) or direct labeling (e.g., direct radiolabeling). In some embodiments, Xaa is a natural amino acid. In certain embodiments, Xaa is a lysine, cysteine, glutamine, tyrosine, histidine, phenylalanine, selenocysteine, or a pyrrolysine. In some embodiments, Xaa is an unnatural amino acid. In certain embodiments, Xaa is ap-acetylphenylalanine, p-azidomethyl-L-phenylalanine, formylglycine, sulfone, maytansine, or a furan-amino acid. In some embodiments, the HER2-binding agent comprises the C-terminal sequence GGC. In some embodiments, the HER2-binding agent comprises the C-terminal sequence GGCG (SEQ ID NO: 201). In some embodiments, the HER2-binding agent comprises the C-terminal sequence GGKG (SEQ ID NO: 202). In some embodiments, the HER2-binding agent comprises the C-terminal sequence GGKGGKG (SEQ ID NO: 203). In some embodiments, the HER2-binding agent comprises the C-terminal sequence GGKGGC (SEQ ID NO: 204).

In some embodiments, the HER2-binding agent contains a protein that contains an additional C-terminal amino acid sequence that enhances tumor cell internalization of the binding agent. In some embodiments, the HER2-binding agent further comprises the structure VHH-IPS, wherein IPS is an internalizing peptide sequence. In some embodiments, the HER2 binding agent further comprises the sequence Argₙ(Rₙ), wherein n=4-16. In some embodiments, the HER2 binding agent further comprises the sequence (ArgLys)ₙ((RK)n), wherein n=2-8. In some embodiments, the HER2-binding agent further comprises a C-terminal amino acid sequence selected from the group: GRKKRRQRRRPPQ (SEQ ID NO: 158), RQIKIWFQNRRMKWKK (SEQ ID NO: 159), GWTLNSAGYLLGKINLKAL AALAKKIL (SEQ ID NO: 160), KLALKLALKALKAALKLA (SEQ ID NO: 161), RRIP (SEQ ID NO: 162), NRRPRR (SEQ ID NO: 163), an amino acid sequence consisting of SEQ ID NOs: 162 and 163, KETWWETWWTEWSQPKKKRKV (SEQ ID NO: 164), PLILLRLLRGQF (SEQ ID NO: 165), PLIYLRLLRGQF (SEQ ID NO: 166), KLWMRWYSPTTRRYG (SEQ ID NO: 167), GINTLQKYYCRVRG (SEQ ID NO: 168), RLWMRWYSPRTRAYGC (SEQ ID NO: 169), GKCSTRGRKCCRRKK (SEQ ID NO: 170), and GRCAVLSCLPKEQI (SEQ ID NO: 171).

In some embodiments, the HER2-binding agent contains a fusion protein comprising two or more VHH domains. In some embodiments, the HER2-binding agent comprises two or more of the same VHH binding domain (e.g., a homodimer, a homotrimer or a homomultimer). In some embodiments, the protein comprises two or more different VHH binding domains (e.g., a heterodimer, a heterotrimer, or a heteromultimer).

In some embodiments, the disclosure provides a multispecific HER2-binding agent that contains a fusion protein comprising at least one HER2-binding VHH domain provided herein and one or more additional binding domains. In some embodiments, one or more of the additional binding domains is an antibody (e.g., a VHH (single domain antibody) or an antigen-binding fragment of an antibody). In some embodiments, one or more of the additional binding domains is a VHH binding domain.

The provided HER2-binding agents can be monovalent or multivalent for HER2 and/or a different antigen. In some embodiments, the HER2-binding agent is monovalent for HER2 (e.g., contains one HER2-binding domain). In some embodiments, the multivalent HER2-binding agent is multivalent and contains more than one binding domain. In some embodiments, the HER2-binding agent contains two or more copies of a binding domain that specifically binds HER2. In some embodiments, the multivalent HER2-binding agent contains 2, 3, 4, 5, 6, or more copies of a HER2-binding VHH domain. In some embodiments, the HER2-binding agent contains two or more copies of different binding domains (e.g., VHH binding domains) that specifically bind HER2. In some embodiments, the HER2-binding agent contains 2, 3, 4, 5, 6, or more different VHH domains that specifically bind HER2. In some embodiments, the HER2-binding agent contains 2, 3, 4, 5, 6, or more different VHH binding domain that specifically bind different epitopes of HER2.

The provided HER2-binding agents can also be monospecific or multispecific. In some embodiments, the HER2-binding agent is monospecific for HER2 (i.e., contains one or more binding domains that bind to the same epitope of HER2). In certain embodiments, the HER2-binding agent contains 2, 3, 4, 5, 6, or more copies of the same HER2-binding VHH domain. In some embodiments, the HER2-binding agent is multispecific. In certain embodiments, the HER2-binding agent is multispecific and contains one or more binding sites that bind different epitopes of HER2. In certain embodiments, the HER2-binding agent is multispecific and contains 2, 3, 4, 5, 6, or VHH domains that specifically bind different epitopes or domains of HER2 (e.g., HER2 Domain I, II, III, or IV). In some embodiments, the HER2-binding agent is multispecific and contains two or more copies of different binding domains (e.g., VHH binding domains) that specifically bind different antigens (e.g., HER2 and EGFR). In certain embodiments, the HER2-binding agent is multispecific and contains 2, 3, 4, 5, 6, or VHH domains that specifically bind different antigens (e.g., HER2 and EGFR).

In additional embodiments, the provided HER2-binding agents are monospecific and multivalent or the provided HER2-binding agents are multispecific and multivalent. In some embodiments, the provided HER2-binding agent is multivalent and monospecific. In certain embodiments, the HER2-binding agent is multivalent and multispecific and contains 2, 3, 4, 5, 6, or more copies of the same HER2-binding VHH domain. In some embodiments, the provided HER2-binding agent is multivalent and multispecific. In certain embodiments, the HER2-binding agent is multivalent and multispecific and contains 2, 3, 4, 5, 6, or VHH domains that specifically bind different epitopes of HER2. In some embodiments, the HER2-binding agent is multivalent and monospecific and contains two or more copies of different binding domains that specifically bind different antigens (e.g., HER2 and EGFR). In certain embodiments, the HER2-binding agent is multispecific and contains 2, 3, 4, 5, 6, or VHH domains that specifically bind different antigens.

In some embodiments, one or more of the additional binding domains specifically binds a second antigen that is different from HER2. In some embodiments, the second antigen is a tumor associated antigen or tumor microenvironment associated antigen. In some embodiments, the second antigen is an immunomodulatory antigen, wherein the antigen is involved in enhancing or dampening a signaling pathway in an immune cell. In some embodiments, the second antigen is an immunomodulatory antigen, wherein the antigen is involved in enhancing or dampening a signaling pathway in an immune cell.

In some embodiments, the HER2-binding agent contains a fusion protein having the following structure: HER2VHH-Linker-HER2VHH wherein HER2VHH is a HER-2 binding VHH disclosed herein and the Linker is a linker provided herein or otherwise known in the art.

In some embodiments, the HER2-binding agent contains a fusion protein comprising a HER2-binding VHH domain operably linked to an IgG Fc. In certain embodiments, the fusion protein is bivalent having two VHH domains that specifically bind HER2 (e.g., having 2 HER2-binding VHH domains per molecule, and having the structure (HER2VHH-Linker-HER2VHH-Linker-Fc). In some embodiments, the fusion protein is tetravalent (e.g., having 4 HER2-binding VHH domains per molecule, and having the structure HER2VHH-Linker-HER2VHH-Linker-HER2VHH-Linker-HER2VHH-Linker-Fc) or hexavalent (e.g., having 4 HER2-binding VHH domains per molecule, and having the structure HER2VHH-Linker-HER2VHH-Linker-HER2VHH-Linker-HER2VHH-Linker-HER2VHH-Linker-HER2VHH-Linker-Fc).

In some embodiments, the HER2-binding agent contains a fusion protein that comprises a HER2-binding VHH domain disclosed herein fused to a heterologous amino acid sequence. The fusion of the HER2-binding VHH domain to the heterologous polypeptide sequence can be at any convenient site on the protein and may be N-, C- and/or N-/C-terminal fusion(s). In some embodiments, the heterologous polypeptide sequence is fused to the N-terminus of the HER2-binding VHH domain. In some embodiments, the heterologous polypeptide sequence is fused to the C-terminus of the HER2-binding VHH domain. In certain embodiments, a heterologous polypeptide sequence is fused to the N-terminus of the HER2-binding VHH domain and a heterologous polypeptide sequence is fused to the C-terminus of the HER2-binding VHH domain. Such fusion proteins may be prepared by any suitable route, including by recombinant expression, chemical linkage, enzymatic ligation (e.g., sortase A) or other methods known in the art.

In some embodiments, the heterologous amino acid sequence is a toxin. In some embodiments, the heterologous amino acid sequence is a label.

In some embodiments, the HER2-binding VHH domain is fused (operably linked) to a heterologous polypeptide having biological activity. In some embodiments, the molecule having biological activity is a peptide or protein such as an enzyme, immunoglobulin, cytokine, an anti-cancer agent or a fragment or variant thereof. Alternatively, the biologically active component of the fusion protein may an anti-cancer drug, an NSAID, a steroid, an analgesic, a toxin or other pharmaceutically active agent. Anti-cancer drugs may include cytotoxic or cytostatic drugs.

In some embodiments, a molecule having biological activity in the fusion protein is a therapeutic agent, e.g., an anti-inflammatory drug (e.g., a steroid or a non-steroidal anti-inflammatory drug), an anti-cancer drug, a cytotoxic agent (e.g., a toxin, such as cholera toxin, or a radionuclide comprising a radioactive element for therapeutic or diagnostic use), or a cytostatic or an analgesic agent.

In some embodiments, the HER2-binding agent contains a fusion protein comprising a HER2-binding VHH domain provided herein fused to another immunoglobulin variable or constant region, or another VHH domain having specific binding affinity for HER2 or another antigen. In some embodiments, the fusion protein contains dimers, trimers, tetramers, or higher multimers (i.e., pentamers, hexamers, heptamers, octamers, nonamers, or decamers, or greater) of VHH binding domains.

In some embodiments, the HER2-binding VHH domain is fused to a heterologous amino acid sequence comprising an immunoglobulin domain. In some embodiments, the HER2-binding VHH domain is fused to a heterologous amino acid sequence of an antibody, or an engineered fragment thereof, including Fab, Fc, F(ab′)₂ (including chemically linked F(ab′)₂ chains), Fab′, scFv (including multimer forms thereof, i.e., di-scFv, or tri-scFv), or BiTE (bi-specific T-cell engager)). Antibody fragments also include variable domains and fragments thereof, as well as other single domain antibody type fragments. In some embodiments, the fusion protein is a chimeric antigen receptor. In some embodiments, the immunoglobulin Fc region is of an IgG isotype selected from the group consisting of IgG1 isotype, IgG2 isotype, IgG3 isotype, and IgG4 isotype.

In some embodiments, the HER2-binding VHH domain is fused to a heterologous amino acid sequence comprising an Fc domain or a portion thereof (such as a human Fc). In certain embodiments, the Fc portion increases the half-life and/or production of a polypeptide comprising the HER2-binding domain. In some embodiments, the Fc domain or portion thereof increases the half-life of a polypeptide comprising the HER2-binding domain. In some embodiments, the multispecific HER2-binding agent further comprises an Fc domain, such as described herein. In some embodiments, the multispecific HER2-binding agent comprises at least one VHH domain that binds HER2, at least one additional binding domain that binds a different epitope or domain of HER2 (e.g., HER2 Domain I, II, III, or IV) and an Fc domain. In some embodiments, the multispecific HER2-binding agent comprises at least one VHH domain that binds HER2, at least one additional binding domain that binds a different antigen, and an Fc domain. In some embodiments, one or more single domain antibodies of the disclosure may be linked (optionally via a suitable linker or hinge region) to one or more constant domains (for example, 2 or 3 constant domains that can be used as part of/to form an Fc portion), to an Fc portion and/or to one or more antibody parts, fragments or domains that confer one or more effector functions to the polypeptide of the disclosure and/or may confer the ability to bind to one or more Fc receptors. For example, for this purpose, and without being limited thereto, the one or more further amino acid sequences may comprise one or more CH2 and/or CH3 domains of an antibody, such as from a heavy chain antibody of from a conventional human chain antibody; and/or may form an Fc region, for example from IgG (e.g., from IgG1, IgG2, IgG3 or IgG4), from IgE or from another human Ig such as IgA, IgD or IgM. For example, Intl. Publ. No. WO1994/04678 describes heavy chain antibodies comprising a Camelid VHH domain or a humanized derivative thereof, in which the Camelidae CH2 and/or CH3 domain have been replaced by human CH2 and CH3 domains, so as to provide an immunoglobulin that consists of two heavy chains each comprising a single domain antibody and human CH2 and CH3 domains (but no CH1 domain), which has the effector function provided by the CH2 and CH3 domains and can function without the presence of any light chains.

In some embodiments, the multispecific HER2-binding agent is a bispecific construct that comprises at least one HER2-binding VHH domain provided herein, and at least one additional binding domain capable of binding to a surface molecule expressed on an immune effector cell (e.g., a T cell). In some embodiments, the surface molecule is an activating component of a T cell, such as a component of the T cell receptor complex. In some embodiments, the surface molecule is an activating T cell antigen that is expressed on a T cell and is capable of inducing T cell activation upon interaction with an antigen binding molecule. For example, in some embodiments, interaction of an antigen binding molecule with an activating T cell antigen may induce T cell activation by triggering the signaling cascade of the T cell receptor complex. In some embodiments, the activating T cell antigen is CD3 or CD2. In some embodiments, a provided bispecific HER2-binding agent is capable of specifically binding an activating T cell antigen expressed on a human T cell, such as human CD3 or human CD2. In particular embodiments, the additional binding domain that is specific to the activating T cell antigen (e.g., CD3 or CD2) is an antibody (e.g., a VHH (single domain antibody)) or an antigen-binding fragment of an antibody. In some embodiments, the one or more of the additional binding domain that is specific to the activating T cell antigen is a VHH domain or an antigen-binding fragment or variant of a VHH domain.

In some embodiments, the provided HER2-binding agent is a bispecific antibody T cell-engager comprising at least one HER2-binding VHH domain provided herein and an additional binding domain that is an antibody (e.g., a VHH (single domain antibody)) or antigen-binding fragment specific for an activating component of a T cell (e.g., a T cell surface molecule, e.g., CD3, a CD3 complex, or CD2).

### Linkers

The polypeptide components of the fusion proteins contained in the HER2-binding agents provided herein can be directly fused (operably linked) via a linker moiety to other components of the fusion protein. In some embodiments, the linker is a peptide, peptide nucleic acid, or polyamide linkage.

In some embodiments, the HER2-binding agent contains a protein that is a fusion protein comprising a HER2-binding VHH domain fused to one or more polypeptide sequences via one or more polypeptide linkers. In some embodiments, the linker is a flexible linker. In some embodiments, the linker is a rigid linker. Suitable peptide linkers include a plurality of amino acid residues, for example, 4, 5, 6, 7, 8, 9, 10, 15, 20, 25, or 30 amino acids. In some embodiments, the linkers are composed predominately of the amino acids Glycine and Serine, denoted as GS-linkers herein. The GS-linkers can be of various lengths, for example, 4, 5, 6, 7, 8, 9, 10, 11, 12, 13, 14, 15, 16, 17, 18, 19, 20 or 25 amino acids in length. In some embodiments, the GS-linker comprises an amino acid sequence selected from the group consisting of GGSGGS (SEQ ID NO: 172); GGSGGSGGS (SEQ ID NO: 173); GGSGGSGGSGGS (SEQ ID NO: 174); and GGSGGSGGSGGSGGS (SEQ ID NO: 175). In some embodiments, the linker is a flexible linker comprising Glycine residues, such as, by way of non-limiting example, GG, GGG, GGGG (SEQ ID NO: 176), GGGGG (SEQ ID NO: 177), and GGGGGG (SEQ ID NO: 178). In some embodiments, the linker is (GGGGS)n, wherein n is 1 to 5 (SEQ ID NO: 179); (GGGGGS)n, wherein n is 1 to 4 (SEQ ID NO: 180); GGGGS (SEQ ID NO: 181); GGGGGS (SEQ ID NO: 182); GGGGGSGGGGGSGGGGGS (SEQ ID NO: 183); GGGGSG GGGSGGGGS (SEQ ID NO: 184); GGSGGGGSGGGGSGGGGS (SEQ ID NO: 185); or PGGGG (SEQ ID NO: 186). In some embodiments, the HER2-binding agent comprises a combination of a GS-linker and a Glycine linker.

In some embodiments, the HER2-binding agent contains a protein that is a fusion protein comprising a HER2-binding VHH domain fused to one or more polypeptide sequences via one or more polypeptide linkers that comprise the sequence Gn, wherein n=2-8. In some embodiments, the linker comprises the sequence (GS)n, (GGS)n, (GGGS)n (SEQ ID NO: 187), or (GGGGS)n (SEQ ID NO: 188), wherein n=1-16. In some embodiments, the linker comprises the sequence (GGGGS)₃ (SEQ ID NO: 189). In some embodiments, the linker comprises the sequence (EGKSSGSGSESKST)n (SEQ ID NO: 190) or (KESGSVSSEQLAQFRSLD)n (SEQ ID NO: 191), wherein n=1-4. In some embodiments, the linker comprises the sequence GGGSLVPRGSGGGS (SEQ ID NO: 192) or (GAGSAAGSGEF)n (SEQ ID NO: 193), wherein n=1-5. In some embodiments, the linker comprises the sequence (GAGAGX)n (SEQ ID NO: 194), wherein X=A, Y, V, or S (SEQ ID NO: 195), and n=1-16. In some embodiments, the linker comprises the sequence (VPGXG)n, wherein X=A, V, G, or S (SEQ ID NO: 196), and n=1-40.

In some embodiments, the HER2-binding agent contains a protein that is a fusion protein comprising a HER2-binding VHH domain fused to one or more polypeptide sequences via one or more polypeptide linkers that are rigid. In some embodiments, the linker comprises the sequence (APAP)n (SEQ ID NO: 197), wherein n=10-34. In some embodiments, the linker comprises the sequence (EAAAK)n (SEQ ID NO: 198), wherein n=1-3. In some embodiments, the linker comprises the sequence A(EAAAK)₄ALEA(EAAAK)₄A (SEQ ID NO: 199). In some embodiments, the linker comprises the sequence (A)₃(H)₆ (SEQ ID NO: 200).

In some embodiments, the linker is a cross-linkable linker. In certain embodiments the linker is PEG, POEGMA, or a carbonyl chain.

### Chimeric Receptors and Engineered Cells

In some embodiments, the disclosure provides a chimeric antigen receptor (CAR) having an extracellular domain that comprises one or more HER2-binding VHH domains provided herein. CAR constructs provided herein include an extracellular domain comprising one or more HER2-binding VHH domains provided herein, a transmembrane domain and an intracellular signaling region. The one or more HER2-binding VHH domains contained in the extracellular antigen binding unit of the CAR “bind” or are “capable of binding” HER2 with sufficient affinity such that the CAR is useful in therapy in targeting a cell or tissue expressing HER2.

CARs are synthetic receptors typically containing an extracellular targeting/binding moiety that is associated with one or more signaling domains in a single fusion molecule, and that are expressed on the surface of an immune effector cell, such as a T cell. Thus, CARs combine antigen-specificity and T cell activating properties in a single fusion molecule. In some embodiments, the CAR contains the signaling domain of a costimulatory molecule, such as CD28, and CD3 zeta to provide dual signaling to direct combined activating and co-stimulatory signals. In some embodiments, the CAR comprises three or more signaling domains.

In some embodiments, the CAR contains an antigen binding domain that comprises two HER2-binding VHH domains, thus providing a bivalent binding molecule. In certain embodiments, the antigen binding domain comprises two or more of the same HER2-binding VHH domains. In certain embodiments, the antigen binding domain comprises two or more different HER2-binding VHH domains. In certain embodiments, the antigen binding domain comprises two or more HER2-binding VHH domains that bind different epitopes of HER2. In certain embodiments, the antigen binding domain comprises one or more HER2-binding VHH domains and one or more VHH binding domains that specifically binds a different antigen.

In particular embodiments, the CAR further comprises a hinge or spacer region which connects the extracellular antigen binding domain and the transmembrane domain. This hinge or spacer region can be used to achieve different lengths and flexibility of the resulting CAR. Examples of the hinge or spacer region that can be used include, but are not limited to, Fc fragments of antibodies or fragments or derivatives thereof, hinge regions of antibodies or fragments or derivatives thereof, CH2 regions of antibodies, CH3 regions of antibodies, artificial spacer sequences (for example, peptide sequences), or combinations thereof. Other hinge or spacer regions will be apparent to those of skill in the art and may be used. In some embodiments, the hinge is an IgG4 hinge or a CD8A hinge. In some embodiments, the spacer and transmembrane domain are the hinge and transmembrane domain derived from CD8.

The transmembrane domain of a CAR provided herein is a domain that typically crosses or is capable of crossing or spanning the plasma membrane and is connected, directly or indirectly (e.g., via a spacer, such as an immunoglobulin hinge sequence) to the extracellular antigen binding domain and the endoplasmic portion containing the intracellular signaling domain. In some embodiments, the transmembrane domain of the CAR is a transmembrane region of a transmembrane protein (for example, a Type I transmembrane protein), an artificial hydrophobic sequence or a combination thereof. In certain embodiments, the transmembrane domain comprises the CD3 zeta domain or CD28 transmembrane domain. Other transmembrane domains will be apparent to those of skill in the art and may be used in connection with embodiments of a CAR provided herein.

The intracellular signaling region of a CAR provided herein contains one or more intracellular signaling domain that transmits a signal to a T cell upon engagement of the antigen binding domain of the CAR, such as upon binding antigen. In some embodiments, the intracellular region contains an intracellular signaling domain that is or contains an ITAM signaling domain. Exemplary intracellular signaling domains include, for example, a signaling domain derived from zeta chain of the T-cell receptor complex or any of its homologs (e.g., η chain, FcRIγ and R chains, MB 1 (Iga) chain, and B29 (Ig) chain), human CD3 zeta chain, CD3 polypeptides (delta, gamma or epsilon), syk family tyrosine kinases (e.g., Syk or ZAP 70), src family tyrosine kinases (e.g., Lck, Fyn, or Lyn) and other molecules involved in T-cell transduction, such as CD2, CD5, OX40 and CD28. In particular embodiments, the intracellular signaling region contains an intracellular signaling domain derived from the human CD3 zeta chain.

In some embodiments, the endoplasmic portion of the CAR comprises a CD3 zeta signaling domain. In some embodiments, the costimulatory signaling domain is derived from CD28 or 4-1BB and retains the activity of T cell costimulatory signaling.

In some embodiments, the disclosure provides an isolated nucleic acid construct comprising at least one nucleic acid encoding a CAR as provided herein. In some embodiments, the construct is an expression vector for expression of the CAR in a cell. In some embodiments, the expression vector is a viral vector (e.g., a lentivirus vector). Viral vector technology is well known in the art and is described, for example, in Sambrook et al., (Molecular Cloning: A Laboratory Manual, Cold Spring Harbor Laboratory, New York, 2013). A number of viral based systems have been developed for gene transfer into mammalian cells and are known in the art.

In some embodiments, the disclosure provides an isolated cell or cell population comprising one or more nucleic acid construct as described herein. Also provided is an isolated cell or cell population that has been genetically modified to express a CAR provided herein. Thus, provided herein are genetically engineered cells which comprise, such as stably express, a CAR provided herein. In some embodiments, the cell is selected from the group consisting of a T cell, a Natural Killer (NK) cell, a cytotoxic T lymphocyte (CTL), a regulatory T cell, hematopoietic stem cells and/or pluripotent embryonic/induced stem cells. In some cases, the cell is a T cell, such as a CD4 and/or CD8 T cell. In some embodiments, the cells are autologous to the subject. For example, in some embodiments, T cells may be isolated from a patient (also called primary T cells) for engineering, e.g., transfection or transduction, with a CAR nucleic acid construct.

In particular embodiments, primary T-cells can be purified ex vivo (CD4 cells or CD8 cells or both) and stimulated with a TCR/CD28 agonists, such as anti-CD3/anti-CD28 coated beads. After a two- or three-day activation process, a recombinant expression vector encoding the CAR can be stably introduced into the primary T cells through standard lentiviral or retroviral transduction protocols or plasmid electroporation strategies. Cells can be monitored for CAR expression by, for example, flow cytometry using anti-epitope tag or antibodies that cross-react with native parental molecule. T cells that express the CAR can be enriched through sorting with anti-epitope tag antibodies or enriched for high or low expression depending on the application.

The CAR engineered T cells can be assayed for appropriate function by a variety of means. In some cases, in vitro cytotoxicity, proliferation, or cytokine assays (e.g., IFN-gamma expression) can be used to assess the function of engineered T cells. Exemplary standard endpoints are percent lysis of a tumor line, proliferation of the engineered T cell, or IFN-gamma protein expression in culture supernatant. In some cases, the ability to stimulate activation of T cells upon stimulation of the CAR, e.g., via antigen, can be assessed, such as by monitoring expression of activation markers such as CD69, CD44, or CD62L, proliferation and/or cytokine production.

### II. Nucleic Acids

In some embodiments, the disclosure provides nucleic acids encoding the HER2-binding agents provided herein.

In some embodiments, the disclosure provides an isolated nucleic acid encoding a HER2-binding VHH domain comprising:
(a) a VHH amino acid sequence disclosed in Table 2; or
(b) a VHH sequence selected from SEQ ID NOs: 1-81 and 205.

In some embodiments, the disclosure provides an isolated nucleic acid encoding a HER2-binding VHH domain comprising:
(a) an amino acid sequence having at least 80%, 85%, 90%, 95% 96%, 97%, 98% or 99% sequence identity with a VHH sequence disclosed in Table 2; or
(b) an amino acid sequence having at least 80%, 85%, 90%, 95% 96%, 97%, 98% or 99% sequence identity with a VHH sequence selected from SEQ ID NOs: 1-81 and 205.

In some embodiments, the disclosure provides an isolated nucleic acid encoding a HER2-binding VHH domain comprising:
(a) an amino acid sequence that has 1-25, 1-20, 1-15, 1-10, 1-5 or 1-3 additions, substitutions or deletions compared to a reference VHH sequence disclosed in Table 2; or
(b) an amino acid sequence that has 1-25, 1-20, 1-15, 1-10, 1-5 or 1-3 additions, substitutions or deletions compared to a reference VHH sequence selected from SEQ ID NOs: 1-81 and 205.

In some embodiments, the disclosure provides an isolated nucleic acid encoding a HER2-binding VHH domain comprising:
(a) a complementarity determining region (CDR) 1 comprising an amino acid sequence selected from SEQ ID NOs: 82-101; a CDR2 comprising an amino acid sequence selected from SEQ ID NOs: 102-123; and a CDR3 comprising an amino acid sequence selected from SEQ ID NOs: 124-157 and 206; or
(b) a CDR1, CDR2 and CDR3 comprising the amino acid sequence of a CDR1, CDR2 and CDR3 contained in a VHH sequence disclosed in Table 2.

In some embodiments, the disclosure provides an isolated nucleic acid encoding a HER2-binding VHH domain comprising:
(a) a CDR1 comprising the amino acid sequence of SEQ ID NO: 101; a CDR2 comprising the amino acid sequence of SEQ ID NO: 111; and a CDR3 comprising the amino acid sequence of SEQ ID NO: 135 or 136;
(b) a CDR1 comprising the amino acid sequence of SEQ ID NO: 101; a CDR2 comprising the amino acid sequence of SEQ ID NO: 111; and a CDR3 comprising the amino acid sequence of SEQ ID NO: 135;
(c) a CDR1 comprising the amino acid sequence of SEQ ID NO: 101; a CDR2 comprising the amino acid sequence of SEQ ID NO: 111; and a CDR3 comprising the amino acid sequence of SEQ ID NO: 136;
(d) a CDR1 comprising the amino acid sequence of SEQ ID NO: 82; a CDR2 comprising the amino acid sequence of SEQ ID NO: 102; and a CDR3 comprising the amino acid sequence of SEQ ID NO: 124, 145, 146 or 147;
(e) a CDR1 comprising the amino acid sequence of SEQ ID NO: 83; a CDR2 comprising the amino acid sequence of SEQ ID NO: 103; and a CDR3 comprising the amino acid sequence of SEQ ID NO: 125;
(f) a CDR1 comprising the amino acid sequence of SEQ ID NO: 84; a CDR2 comprising the amino acid sequence of SEQ ID NO: 104; and a CDR3 comprising the amino acid sequence of SEQ ID NO: 126;
(g) a CDR1 comprising the amino acid sequence of SEQ ID NO: 85; a CDR2 comprising the amino acid sequence of SEQ ID NO: 105; and a CDR3 comprising the amino acid sequence of SEQ ID NO: 127;
(h) a CDR1 comprising the amino acid sequence of SEQ ID NO: 86; a CDR2 comprising the amino acid sequence of SEQ ID NO: 106; and a CDR3 comprising the amino acid sequence of SEQ ID NO: 128, 130, 134 or 155;
(i) a CDR1 comprising the amino acid sequence of SEQ ID NO: 87; a CDR2 comprising the amino acid sequence selected from SEQ ID NO: 117; and a CDR3 comprising the amino acid sequence of SEQ ID NO: 129;
(j) a CDR1 comprising the amino acid sequence of SEQ ID NO: 88; a CDR2 comprising the amino acid sequence of SEQ ID NO: 118; and a CDR3 comprising the amino acid sequence selected from SEQ ID NO: 130;
(k) a CDR1 comprising the amino acid sequence of SEQ ID NO: 89; a CDR2 comprising the amino acid sequence of SEQ ID NO: 108 or 112; and a CDR3 comprising the amino acid sequence of SEQ ID NO: 131 or 157;
(l) a CDR1 comprising the amino acid sequence of SEQ ID NO: 89; a CDR2 comprising the amino acid sequence of SEQ ID NO: 108; and a CDR3 comprising the amino acid sequence of SEQ ID NO: 131;
(m) a CDR1 comprising the amino acid sequence of SEQ ID NO: 89; a CDR2 comprising the amino acid sequence of SEQ ID NO: 108 or 112; and a CDR3 comprising the amino acid sequence of SEQ ID NO: 156;
(n) a CDR1 comprising the amino acid sequence of SEQ ID NO: 89; a CDR2 comprising the amino acid sequence of SEQ ID NO: 108 or 112; and a CDR3 comprising the amino acid sequence of SEQ ID NO: 157;
(o) a CDR1 comprising the amino acid sequence of SEQ ID NO: 90; a CDR2 comprising the amino acid sequence of SEQ ID NO: 109 and a CDR3 comprising the amino acid sequence of SEQ ID NO: 132;
(p) a CDR1 comprising the amino acid sequence of SEQ ID NO: 91; a CDR2 comprising the amino acid sequence of SEQ ID NO: 119 and a CDR3 comprising the amino acid sequence of SEQ ID NO: 137;
(q) a CDR1 comprising the amino acid sequence of SEQ ID NO: 92; a CDR2 comprising the amino acid sequence of SEQ ID NO: 120 and a CDR3 comprising the amino acid sequence of SEQ ID NO: 138;
(r) a CDR1 comprising the amino acid sequence of SEQ ID NO: 93; a CDR2 comprising the amino acid sequence of SEQ ID NO: 113 or 114 and a CDR3 comprising the amino acid sequence of SEQ ID NO: 139;
(s) a CDR1 comprising the amino acid sequence of SEQ ID NO: 93; a CDR2 comprising the amino acid sequence of SEQ ID NO: 114 and a CDR3 comprising the amino acid sequence of SEQ ID NO: 206;
(t) a CDR1 comprising the amino acid sequence of SEQ ID NO: 94; a CDR2 comprising the amino acid sequence of SEQ ID NO: 115 and a CDR3 comprising the amino acid sequence of SEQ ID NO: 140 or 149;
(u) a CDR1 comprising the amino acid sequence of SEQ ID NO: 95; a CDR2 comprising the amino acid sequence of SEQ ID NO: 121 and a CDR3 comprising the amino acid sequence of SEQ ID NO: 141;
(v) a CDR1 comprising the amino acid sequence of SEQ ID NO: 96; a CDR2 comprising the amino acid sequence of SEQ ID NO: 122; and a CDR3 comprising the amino acid sequence of SEQ ID NO: 142;
(w) a CDR1 comprising the amino acid sequence of SEQ ID NO: 97; a CDR2 comprising the amino acid sequence of SEQ ID NO: 116; and a CDR3 comprising the amino acid sequence of SEQ ID NO: 150;
(x) a CDR1 comprising the amino acid sequence of SEQ ID NO: 98; a CDR2 comprising the amino acid sequence of SEQ ID NO: 107; and a CDR3 comprising the amino acid sequence of SEQ ID NO: 144, 151, 152 or 153;
(y) a CDR1 comprising the amino acid sequence of SEQ ID NO: 99; a CDR2 comprising the amino acid sequence of SEQ ID NO: 109; and a CDR3 comprising the amino acid sequence of SEQ ID NO: 132 or 148; or
(z) a CDR1 comprising the amino acid sequence of SEQ ID NO: 100; a CDR2 comprising the amino acid sequence of SEQ ID NO: 110; and a CDR3 comprising the amino acid sequence of SEQ ID NO: 133 or 154;
or
an isolated nucleic acid encoding a HER2-binding VHH domain comprising:
(ab) a CDR1 comprising SEQ ID NO: 97; a CDR2 comprising SEQ ID NO: 123; and a CDR3 comprising SEQ ID NO: 143; or
(ab) a CDR1 comprising SEQ ID NO: 101; a CDR2 comprising SEQ ID NO: 111; and a CDR3 comprising SEQ ID NO: 136.

In some embodiments, the provided nucleic acid sequences encode a fusion protein as provided herein. In some embodiments, the nucleic acid sequence further comprises one or more regulatory sequences that are suitable for replication and/or expression of the encoded protein in a selected host cell (e.g., a transcriptional promoter, operator, or enhancer, or an, mRNA ribosomal binding site). In some embodiments, the nucleic acid further encodes a leader sequence that directs secretion of the protein, which leader sequence is typically cleaved such that it is not present in the secreted protein. The leader sequence may be a native heavy chain (or VHH) leader sequence or may be a heterologous leader sequence. Nucleic acids can be constructed using recombinant DNA techniques and/or chemical synthesis methods known in the art.

In some embodiments, the disclosure provides a vector comprising a nucleic acid encoding a HER2-binding protein provided herein. Vectors generally include a selectable marker and an origin of replication, for propagation in a host. The provided vectors include, but are not limited to, DNA vectors, phage vectors, viral vectors, and retroviral vectors.

In some embodiments, the disclosure provides an expression vector comprising a nucleic acid encoding a HER2-binding protein provided herein. Generally, expression vectors include nucleic acid encoding the HER2-binding protein operably linked to suitable transcriptional or translational regulatory sequences for the host cell in which the protein is to be expressed, such as those derived from a mammalian, microbial, viral, or insect genes. Examples of regulatory sequences include transcriptional promoters, operators, or enhancers, mRNA ribosomal binding sites, and appropriate sequences which control transcription and translation.

A variety of host-vector expression systems can be used to express the protein component of the provided HER2-binding agents. These systems include, but are not limited to, microorganisms such as bacteria transformed with recombinant bacteriophage, plasmid or cosmid DNA expression vectors; yeast transformed with yeast expression vectors (Giga-Hama et al., Biotechnology and Applied Biochemistry (1999) 30(3):235-44); insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid; see, e.g., Babe et al., Canadian Journal of Communication (2000) 25(1):19); or animal cell systems infected with virus expression vectors (e.g., vaccinia virus, adenovirus, etc.). Those of skill in the art are aware of various techniques for optimizing mammalian expression of proteins: see, e.g., Kaufman, Mol Biotechnol. (2000) 16(2):151-60; Colosimo et al., Biotechniques (2000) 29(2):314-31. Mammalian cells that are useful in recombinant protein production include but are not limited to VERO cells, HeLa cells, Chinese hamster ovary (CHO) cell lines COS cells, including COS 7 cells; HEK cells; 293 cells, including 293-6E cells; CHO cells, including CHO-S, DG44. Lec13 CHO cells, and FUT8 CHO cells; PER.C6 cells (Crucell); NSO cell W138, BHK, HepG2, 3T3, RIN, MDCK, A549, PC 12, K562 and 293 cells. Exemplary protocols for the recombinant expression of the HER2-binding polypeptide in bacteria, yeast and other invertebrates are known in the art. In some embodiments, the protein comprising a HER2-binding VHH domain is expressed in yeast (e.g., P. pastoris). See, e.g., U.S. Publ. No. US 2006/0270045 A1. Mammalian host systems for the expression of recombinant proteins also are well known to those of skill in the art. Host cell strains may be chosen for a particular ability to process the expressed protein or produce certain post-translation modifications that are useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein. In some embodiments, a particular eukaryotic host cell is selected based on its ability to make desired post-translational modifications to the polypeptide.

In some embodiments, the disclosure provides a host cell comprising a nucleic acid described herein. In some embodiments, the disclosure provides a host cell comprising a vector containing a nucleic acid disclosed herein. In some embodiments, the disclosure provides a host cell comprising an expression vector disclosed herein. In some embodiments, the host cell is a eukaryotic cell such as a mammalian cell. In some embodiments, the host cell is a prokaryotic cell. In certain embodiments, the host cell expresses a HER2-binding polypeptide disclosed herein.

The introduction of nucleic acids (including vectors) into a desired host cell can be accomplished by any method known in the art, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

### III. Polypeptide Expression and Production

The protein component of a HER2-binding agent provided herein can routinely be produced by conventional recombinant technology and/or peptide synthesis methods known in the art.

In some embodiments, the disclosure provides a recombinant method for producing a protein comprising a HER2-binding VHH domain as provided herein. In some embodiments, the method comprises culturing a host cell containing a nucleic acid encoding a HER2-binding protein under conditions wherein the protein is produced. In some embodiments, the method further comprises recovering the HER2-binding protein produced by the host cell.

In some embodiments, the HER2-binding protein is expressed in a prokaryotic cell, such as a bacterial cell. In some embodiments, the HER2-binding protein is expressed in a eukaryotic cell. In certain embodiments, the HER2-binding protein is expressed in a fungal cell (e.g., such as yeast), a plant cell, an insect cell, or a mammalian cell. Such expression may be carried out, for example, according to procedures known in the art. In some embodiments, the disclosure provides a cell culture media comprising the HER2-binding protein expressed by the cell. In some embodiments, the protein is purified from the cell culture media.

In some embodiments, the HER2-binding VHH domain is produced in a cell-free system. Nonlimiting exemplary cell-free systems are described, for example, in Sitaraman et al., Methods Mol Biol. (2009) 498:229-44; Spirin, Trends Biotechnol. (2004) 22:538-45; Endo et al., Biotechnol Adv. (2003) 21:695-713. In some embodiments, the disclosure provides a cell-free solution comprising the HER2-binding protein. In some embodiments, the protein is purified from the cell-free solution.

As an alternative to recombinant DNA technology, the proteins contained in the HER2-binding agents provided herein and/or the nucleic acid encoding the proteins can be produced through automated polypeptide or nucleic acid synthesis, respectively. In some embodiments, the protein component of a HER2-binding agent provided herein is synthesized in solution or on a solid support in accordance with conventional techniques. Various automatic synthesizers are commercially available and can be used in accordance with known protocols as described in Stewart and Young, Solid Phase Peptide Synthesis, second edition, Pierce Chemical Co., Rockford, Ill., pp. 11 and 12; Tam et al., J Am Chem Soc (1983) 105:6442; Merrifield, Science (1986) 232(4748):341-7; and Barany, G. and Merrifield, R. B., “The Peptides, Vol. 2”, Academic Press, Inc., New York, 1979, pp. 1-284; Gross et al., The Peptides, Analysis, Synthesis, Biology, Vol. 2, Academic Press, 1980, pp. 3-25. The polypeptide components of a HER2-binding agent of the disclosure may also be synthesized by solid-phase technology employing an exemplary peptide synthesizer such as a Model 433A from Applied Biosystems Inc. The purity of any given polypeptide; generated through automated peptide synthesis or through recombinant methods may be determined using reverse phase HPLC analysis. Chemical authenticity of each polypeptide may be established by any method well known to those of skill in the art.

Proteins can be recovered and purified from recombinant cell cultures and other solutions by methods known in the art including but not limited to, ammonium sulphate or ethanol precipitation, acid extraction, anion or cation exchange chromatography, phosphocellulose chromatography, hydrophobic interaction chromatography, affinity chromatography (e.g., using protein A), hydroxyapatite chromatography, lectin and/or heparin chromatography. For therapy, the nucleic acid construct e.g., in the form of a recombinant vector, may be purified by techniques known in the art, such as by means of column chromatography as described in Sambrook et al., Molecular Cloning, a Laboratory Manual, Second Edition, Cold Spring Harbor Laboratory Press, Cold Spring Harbor, N.Y. (1989).

In specific embodiments, it is contemplated that the proteins used in the therapeutic methods of the present disclosure may be modified in order to improve their therapeutic efficacy. Such modification of therapeutic compounds may be used, for example, to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. For example, polyethylene glycol (PEG) has been widely used as a drug carrier, given its high degree of biocompatibility and ease of modification. Attachment of PEG to various drugs has been shown to improve residence time and decrease toxicity.

### IV. Labeled HER2-Binding Agents

### A. Conjugates

In some embodiments, the HER2-binding agents provided herein contain a protein comprising a HER2-binding VHH domain that is conjugated to one or more further moieties via a covalent or non-covalent interaction, to form a HER2-binding agent conjugate. In some embodiments, the HER2-binding agent conjugate contains 1, 2, 3, 4, 5 or more further moieties. In some embodiments, the conjugated moieties of the HER2-binding agent conjugate are the same. In some embodiments, two or more of the conjugated moieties of the HER2-binding agent conjugate are different.

The moieties of the provided HER2-binding agent conjugates can be attached to a protein comprising one or more VHH domains that specifically bind HER2 using any of the various molecular biological or chemical conjugation and linkage methods known in the art. For example, conjugates of a protein comprising at least one HER2-binding VHH domain provided herein and cytotoxic agent(s) can routinely be made using any of a variety of bifunctional protein-coupling agents known in the art, such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl substrate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, linkers such as peptide linkers, cleavable linkers, non-cleavable linkers or linkers that aid in the conjugation reaction, are used to link or conjugate the HER2-binding protein to the moiety (e.g., an effector moiety such as a drug moiety or a radionuclide). In some embodiments, one or more moieties of the provided HER2-binding agent conjugates are attached to a protein comprising one or more VHH domains that specifically bind HER2 by a linker that comprises amino acid residues. Exemplary amino acid linker components include, but are not limited to, a dipeptide, a tripeptide, a tetrapeptide or a pentapeptide. Exemplary dipeptides include, but are not limited to, valine-citrulline and alanine-phenylalanine. Exemplary tripeptides include, but are not limited to, glycine-valine-citrulline and glycine-glycine-glycine. Amino acid residues that can comprise an amino acid linker component can include those occurring naturally, as well as minor amino acids and non-naturally occurring amino acid analogs, such as citrulline. In some embodiments, the amino acid linker component of a HER2-binding agent conjugate provided herein is designed in its selectivity for enzymatic cleavage by a particular enzyme (e.g., a tumor-associated protease, cathepsin B, C and D, and a plasmin protease). Additional exemplary linker components of the provided HER2-binding agent conjugates include, but are not limited to, 6-maleimidocaproyl (“MC”), maleimidopropanoyl (“MP”), valine-citrulline (“val-cit”), a alanine-phenylalanine (“ala-phe”), p-aminobenzyloxycarbonyl (“PAB”), N-Succinimidyl 4-(2-pyridylthio)-pentanoate (“SPP”), N-Succinimidyl 4-(N-maleimidomethyl)cyclohexane-I carboxylate (“SMCC”), and N-Succinimidyl (4-iodo-acetyl)aminobenzoate (“SIAB”).

In certain embodiments, the linker can be composed of one or more linker components. For covalent attachment of the protein and drug moiety the linker typically has two reactive functional groups, i.e., bivalency in a reactive sense. Bivalent linker reagents useful to attach two or more functional or biologically active moieties, such as peptides, nucleic acids, drugs, toxins, antibodies, haptens, and reporter groups and their methods of use in conjugation are known in the art.

In some embodiments, the protein of the HER2-binding agent conjugate comprises at least one HER2-binding VHH domain and the protein is conjugated to a drug moiety. In certain embodiments, the protein of the HER2-binding agent conjugate is conjugated to about 1 to about 20 drug moieties. In some embodiments, the protein of the HER2-binding agent conjugate comprises at least one HER2-binding VHH domain and the protein is conjugated to about 1 to about 20 drug moieties through a linker (L). In some embodiments, the HER2-binding agent conjugate comprises the following components: (VHH domain), (L)_{q} and (moiety)ₘ, wherein the VHH domain is any of the HER2-binding VHH domains provided herein; L is a linker for linking a protein of the HER2-binding agent to the moiety; m is at least 1; q is 0 or more; and the resulting HER2-binding conjugate binds to HER2. In particular embodiments, m is 1 to 4 and q is 0 to 8.

In some embodiments, the disclosure provides a HER2-binding agent conjugate comprising at least one HER2-binding VHH domain provided herein that is conjugated/attached to a therapeutic agent, such as a cytotoxic agent a cytostatic agent. In some embodiments, the therapeutic agent is a targeting moiety, a small molecule drug (a non-polypeptide drug of less than 500 Daltons), a toxin (e.g., an enzymatically active toxin of bacterial, fungal, plant, or animal origin, or fragments thereof), a cytostatic agent, a cytotoxic agent, an immunosuppressive agent, a radioactive isotope (i.e., a radioconjugate), a prodrug-activating enzyme, or an agent that increases biological half-life.

In some embodiments, the HER2-binding agent conjugate is a drug conjugate (i.e., an antibody drug conjugate (ADC)) comprising one or more HER2-binding domain provided herein conjugated to a therapeutic agent, which is either cytotoxic, cytostatic or otherwise provides some therapeutic benefit. In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

In some embodiments, the HER2-binding agent conjugate comprises at least one HER2-binding VHH domain conjugated with a toxin. In some embodiments, the toxin exerts its cytotoxic and/or cytostatic effect by tubulin binding, DNA binding, or topoisomerase inhibition. In some embodiments, the toxin is a bacterial toxin such as diphtheria toxin, a plant toxin such as ricin, a small molecule toxin such as geldanamycin, a maytansinoid, or calicheamicin.

In some embodiments, the HER2-binding agent conjugate comprises at least one HER2-binding VHH domain conjugated with a chemotherapeutic agent. In certain embodiments, the chemotherapeutic agent is selected from daunomycin, doxorubicin, methotrexate, and vindesine.

In some embodiments, the HER2-binding antibody drug conjugates of the disclosure provide for the targeted-delivery of their conjugated therapeutic agents to tumors. In some cases, this results in targeted killing of the tumor cell. In some embodiments, the HER2-binding antibody drug conjugate is internalized in a cell. In some embodiments, the conjugated therapeutic of the ADC has an intracellular activity. In certain embodiments, the HER2-binding agent conjugate is internalized and the therapeutic agent is a cytotoxin that blocks the protein synthesis of the cell. In certain embodiments, the HER2-binding agent conjugate is internalized and the therapeutic agent is a cytotoxin that elicits a cytotoxic and cytostatic effects by a mechanism selected from tubulin binding, DNA binding, and topoisomerase inhibition. In some embodiments, the therapeutic agent is a cytotoxin comprising a polypeptide having ribosome-inactivating activity. In certain embodiments, the therapeutic agent is selected from gelonin, bouganin, saporin, ricin, ricin A chain, bryodin, diphtheria toxin, restrictocin, and Pseudomonas exotoxin A, or a variant thereof.

In some embodiments, the HER2-binding agent contains a HER2-binding protein that is directly or indirectly conjugated with a label to facilitate treatment and/or detection of the bound or unbound HER2-binding agent. Suitable label substances include but are not limited to various enzymes, prosthetic groups, fluorescent materials, luminescent materials, imaging agents, metal ions, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹²⁴I, ¹³¹I, ¹⁷⁷Lu, ¹⁶⁶Ho, ¹⁵³Sm, ¹⁸F, ⁸⁹Zr, ⁶⁷Ga, ⁶⁸Ga, ⁶⁴Cu, and ⁵²Mn. For example, in some embodiments, the label is a radio-opaque or a radioisotope, such as ³H, ¹⁴C, ³²P, ³⁵S, ¹²³I, ¹²⁵I, ¹³¹I. In some embodiments, the label is a radioactive atom for scintigraphic studies, such as ⁹⁹Tc or ¹²³I, or a spin label for nuclear magnetic resonance (NMR/MRI) imaging, such as ⁸⁹Zr, ¹²³I, ¹³¹I, ¹¹¹In, ¹⁹F, ¹³C, ¹⁵N, ¹⁷O, gadolinium, manganese or iron.

In particular embodiments, the label is a radionuclide. In some embodiments, the radionuclide is a radiohalogen isotope. In certain embodiments, the radionuclide is a radiohalogen isotope selected from: ¹⁸F, ⁷⁶Br, ¹²³I, ¹²⁴I, ¹²⁵I, and ¹³¹I or ⁷⁵Br, ⁷⁷Br, ¹²²I, ¹²⁴I, ¹²⁵I, ¹³¹I, and ²¹¹At. In particular embodiments, the radionuclide is ¹³¹I, ²¹¹At, ¹²⁵I, or ¹²⁴I. In some embodiments, the radionuclide is a radiometal isotope. In certain embodiments, the radionuclide is a member radiometal isotope selected from ¹⁴⁴Sc, ⁴⁵Ti, ⁵¹Cr, ⁶²Cu, ⁶⁴Cu, ⁶⁶Ga, ⁶⁸Ga, ⁶⁸Ge, ⁷⁵Se, ⁸²Sr, ⁸⁶Y, ⁹⁹Mo, ^{99m}Tc, ^{110m}In, ¹¹¹In, ¹⁶⁶Ho, ¹⁸⁶Re, ^{195m}Pt, and ²⁰¹Tl; or ⁴⁷Sc, ⁵²Mn, ⁶⁴Cu, ⁶⁷Cu, ⁶⁷Ga, ⁸⁹Zr, ⁹⁰Y, ¹¹¹In, ¹⁵³Sm, ¹⁴⁹Tb, ¹⁶¹Tb, ¹⁶⁶Ho, ¹⁷⁷Lu, ¹⁸⁸Re, ²¹²Pb, ²¹²Bi, ²¹³Bi, ²²⁵Ac, ²²⁶Th, and ²²⁷Th. In particular embodiments, the radionuclide is ¹⁷⁷Lu, ¹³¹I, ²¹¹At, or ²²⁵Ac.

In some embodiments, the HER2-binding agent contains a HER2-binding protein that is conjugated to a label via a chelating moiety. In some embodiments, the chelating moiety is covalently linked to the protein via a lysine residue. In some embodiments, the label forms a complex with a metal, and the complex is chelated by the chelating moiety.

The HER2-binding agent conjugates provided herein can be prepared by any of a variety of methods, such as organic chemistry reactions, conditions, and reagents known in the art. In some embodiments, the method includes: (1) reaction of a nucleophilic group of a protein comprising a HER2-binding VHH domain provided herein with a bivalent linker reagent, to form HER2-binding VHH-L, via a covalent bond, followed by reaction with a therapeutic agent moiety T; and (2) reaction of a nucleophilic group of a therapeutic agent moiety with a bivalent linker reagent, to form T-L, via a covalent bond, followed by reaction with the nucleophilic group of the protein comprising the HER2-binding VHH domain.

Nucleophilic groups on proteins include, but are not limited to: (a) N-terminal amine groups, (b) side chain amine groups, e.g., lysine, (c) side chain thiol groups, e.g., cysteine, and (d) sugar hydroxyl or amino groups where the protein is glycosylated. Amine, thiol, and hydroxyl groups are nucleophilic and capable of reacting to form covalent bonds with electrophilic groups on linker moieties and linker reagents including: (a) active esters such as NHS esters, HOBt esters, haloformates, and acid halides; (b) alkyl and benzyl halides such as haloacetamides; (c) aldehydes, ketones, carboxyl, and maleimide groups. Additional nucleophilic groups can be introduced into proteins through the reaction of lysines with 2-iminothiolane (Traut's reagent) resulting in conversion of an amine into a thiol. Reactive thiol groups can be introduced into protein (or fragment thereof) by introducing one, two, three, four, or more cysteine residues (e.g., preparing mutant proteins comprising one or more non-native cysteine amino acid residues).

Conjugates, such as HER2-binding agent conjugates, can also be produced by modification of a protein, such as a HER2-binding protein disclosed herein, to introduce electrophilic moieties, which can react with nucleophilic substituents on the linker reagent or the therapeutic agent. The sugars of glycosylated proteins can be oxidized, e.g., with periodate oxidizing reagents, to form aldehyde or ketone groups which can lead with the amine group of linker reagents or therapeutic agent moieties. The resulting imine Schiff base groups can form a stable linkage, or can be reduced, e.g., by borohydride reagents to form stable amine linkages. In certain embodiments, reaction of the carbohydrate portion of a glycosylated protein with either galactose oxidase or sodium meta-periodate can yield carbonyl (aldehyde and ketone) groups in the protein that can react with appropriate groups on the therapeutic agent moiety (Hermanson, Bioconjugate Techniques). In certain embodiments, proteins containing N-terminal serine or threonine residues can react with sodium meta-periodate, resulting in production of an aldehyde in place of the first amino acid. Such an aldehyde can be reacted with a therapeutic agent moiety or linker nucleophile.

Likewise, nucleophilic groups on a therapeutic agent moiety may include but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (a) active esters such as NHS esters, HOBi esters, haloformates, and acid halides; (b) alkyl and benzyl halides such as haloacetamides; and (c) aldehydes, ketones, carboxyl, and maleimide groups.

The HER2-binding agent conjugates provided herein may be prepared using any method known in the art. See, e.g., Intl. Publ. Nos. WO 2009/067800 and WO 2011/133886, and U.S. Publ. No. 2014322129, each of which is incorporated by reference herein in its entirety

### B. Targeted Radiotherapeutic HER2-Binding Agents

In some embodiments, the disclosure provides HER2-binding targeted radiotherapeutic agents for therapeutic and/or diagnostic purposes comprising a HER2-binding agent provided herein. Such targeted agents may comprise, in addition to a HER2-binding VHH domain described herein, a radiolabeled prosthetic agent/group and optionally, a chelating agent (either macrocyclic or acyclic).

It is noted that, although such agents are described herein as “radiotherapeutic” agents, this term is intended to cover agents suitable for both therapeutic and diagnostic purposes. Such targeted radiotherapeutic agents may comprise, in addition to a HER2-binding agent as described herein (i.e., a targeting moiety), a radiolabeled prosthetic agent/group and, optionally, a chelating agent (either macrocyclic or acyclic). Radiolabeled prosthetic agents are compounds or radicals that generally include a radiolabel, a charged group (CG), and a macromolecule conjugating moiety (MMCM), suitable for attachment to the HER2-binding agent. Each of these components can optionally be associated with one or more cleavable (or non-cleavable) linkers.

Prosthetic agents that can be attached to a HER2-binding agent disclosed herein to provide targeted radiotherapeutic agents are not particularly limited. Generally, any prosthetic agent comprising a radiolabel, wherein the prosthetic agent can be suitably attached to a HER2-binding agent as provided herein, can be used in accordance with the present disclosure.

The radiolabel can, in some embodiments, be a radioactive halogen. Radiohalogens can include, e.g., alpha emitters, beta emitters, or Auger electron emitters. They can include radiohalogens suitable for PET, SPECT, or intra-operative imaging. See, e.g., U.S. Pat. No. 5,302,700, which is incorporated herein by reference in its entirety. The radioactive halogen can be, e.g., selected from the group consisting of ¹⁸F, ⁷⁵Br, ⁷⁶Br, ⁷⁷Br, ¹²³I, ¹²⁴I, ¹²⁵I, ¹³¹I and ²¹¹At. In some embodiments, the radioactive halogen is ¹⁸F. In some embodiments, the radioactive halogen is selected from the group consisting of ⁷⁵Br, ⁷⁶Br, ⁷⁷Br, ¹²³I, ¹²⁴I, ¹²⁵I, ¹³¹I and ²¹¹At. In some embodiments, the radioactive halogen is selected from ¹²³I, ¹²⁴I, ¹²⁵I, ¹³¹I and ²¹¹At. In some embodiments, the radioactive halogen is selected from ¹²³I, ¹²⁴I, ¹²⁵I, and ¹³¹I. In some embodiments, the radioactive halogen is ²¹¹At.

In some embodiments, the radiolabel is a metallic radionuclide. Metallic radionuclides that can be complexed within the prosthetic agent (e.g., within a chelating agent as referenced above) include, but are not limited to, ⁶⁴Cu, ⁶⁷Cu, ⁶⁷Ga, ⁶⁸Ga, ⁸⁹Zr, ⁹⁰Y, ¹¹¹In, ¹⁷⁷Lu, ²¹²P, ²¹²Bi, ²¹³Bi, and ²²⁵Ac.

The charged group (CG) is typically a group that is charged under the physiological conditions of the internal cell environment. In some embodiments, the charged group (CG) comprises a guanidine, a PO₃H group, or an SO₃H group. In some embodiments, CG is a guanidino-alkyl group containing more than one carbon. In some embodiments, CG is a guanidino-hydrophilic group (such as an amino- or hydroxyl-containing group), and/or an alkyloxycarbonylguanidine group. In some embodiments, CG comprises one or more charged D-amino acids such as arginine, glutamate, aspartate, lysine, and/or phosphono/sulfo phenylalanine. In some embodiments, CG comprises a hydrophilic carbohydrate moiety. The compounds, in some embodiments, may contain one, two or three CG moieties (and, optionally, corresponding linker groups) to increase intracellular trapping in cancer cells. The prosthetic groups provided herein can comprise a single CG or may comprise two or more CGs. Where more than one CG is associated with a given prosthetic group, each such CG can be the same or different.

The MMCM is a moiety that can bind to the HER2-binding agent as provided herein. In some embodiments, MMCM is an active ester. An active ester is defined herein as an ester that can be conjugated with a group (e.g., amine group) present on the HER2-binding agent, typically under mild conditions, i.e., conditions that will not result in loss of biological function of the HER2-binding agent. Exemplary such MMCM groups include, but are not limited to, N-hydroxysuccinimide (NHS) or tetrafluorophenol (TFP) ester, pentafluorophenol (PFP), paranitrolphenol (PNP), an isothiocyanate group, or a maleimide group. Such MMCMs generally result in random (non-site specific) labeling of amine groups on the HER2-binding agent. In some embodiments, MMCM is, e.g., the tripeptide GGG which provides for site-specific conjugation to be performed, e.g., using the enzyme sortase, which results in conjugation to only one site (either the N-terminus or the C-terminus of the HER2-binding agent). Enzymes that may be employed for obtaining suitable conjugation include, for example, transglutaminase, lipoic acid ligase, farnesyl transferase, and many more, including those disclosed, e.g., in Massa et al., Exp Opin Drug Del. (2016) 13(8):1149-63; Zhang et al., Chem Soc Rev. (2018) 47:9106-36; Falck et al., Antibodies (2018) 7(4):1-19, and van Berkel et al., Drug Disc. Today: Tech. (2018) 30:3-10, each of which is incorporated herein by reference in its entirety. It is understood that, while the prosthetic agent is referred to as comprising an MMCM and the targeted radiotherapeutic agent is generally described as comprising a prosthetic agent attached to a HER2-binding agent, the targeted radiotherapeutic agent will not typically comprise an intact MMCM; rather, it will comprise a moiety resulting from the coordination of the MMCM to the HER2-binding agent.

Certain, non-limiting prosthetic groups conjugated to a HER2-binding agent to give targeted radiotherapeutic agents encompassed by the present disclosure include, for example, those of Formula 1 and derivatives and variants thereof.

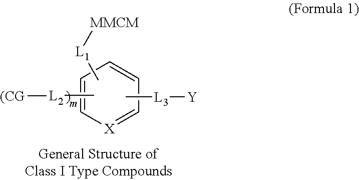

Certain such Class I type compounds are generally disclosed in Intl. Publ. No. WO2018/178936 (Duke University), which is incorporated herein by reference in its entirety. Generally, such prosthetic agents can comprise a homo (X=CH) or hetero (X=N) aromatic ring having attached thereto: a MMCM to couple the prosthetic compound/radical to the HER2-binding agent, a radioactive halogen as described above (Y); and one or more CGs. Each of these components can be attached to the aromatic ring through a linker (L₁, L₂, L₃) or can be directly bonded to the aromatic ring (i.e., where L₁ and/or L₂ and/or L₃ is a bond). Each of these components shown in Formula 1 is described in further detail below.

The charged group (CG) is typically present in the prosthetic groups of Formula 1, i.e., m is 1 or greater. Typically, m is 1; however, more than one CG can be attached to the ring such that m=2, m=3, and (where X=CH), m can be 4. Where more than one CG is attached to the ring, each such CG (and corresponding L₂) can be the same or different. In certain embodiments, as referenced below (as shown in Formula 2), another moiety can be attached to the ring of Formula 1 and, where such additional moiety is charged, m can be 0 (i.e., the additional moiety may, in some embodiments, effectively serve as the “charged group”). CG can be directly bound to the aromatic ring of Formula 1 (L₂=a direct bond) or can be bound to the aromatic ring through a linker (L₂). L₂ can be, e.g., a spacer such as a substituted or unsubstituted alkyl chain (e.g., a simple substituted or unsubstituted alkyl chain such as a methylene), a substituted or unsubstituted alkenyl chain, a substituted or unsubstituted alkynyl chain, a PEG chain of at least three oxygens, or any of the foregoing containing a Brush Border enzyme-cleavable peptide such as GK, GY, or GFK. It is noted that, in certain embodiments, where CG is a guanidine and L₂ is an unsubstituted alkyl chain, the unsubstituted alkyl chain comprises two or more carbon atoms.

In some embodiments, a metabolizable spacer or cleavable linker, L₂ (e.g., a Brush Border enzyme cleavable linker), is located between CG and the aromatic ring of Formula 1. With these formulations, increased uptake and retention of radioactivity in the kidneys can be avoided as the CG moiety is cleaved off in the kidneys, eliminating the charge and allowing the radioactive species (now neutral or less charged) to escape from the renal tubule cells in the kidney after which they are rapidly excreted into the urine. While Brush Border enzyme cleavable linkers have been used before with radioactivity, they have not been used in this way to create a “charge switch” where the labeling reagent is charged in the tumor so is retained but loses charge in the kidney, so it is cleared.

Such linkers include linker sequences targeting meprin β, a metalloprotease expressed in the kidney brush-border membrane (Jodal et al., PLoS One (2015) 10(4):e0123443); C-terminal lysines linked to antibody fragments via the epsilon-amino group of lysine or a C-terminal (N(epsilon)-amino-1,6-hexane-bis-vinyl sulfone)lysine that show reduced kidney uptake by taking advantage of the lysine specific carboxypeptidase activity of the kidney brush border enzymes that cleave off the radiolabeled peptide linker prior to uptake by proximal tubule cells (Li et al., Bioconjugate Chem. (2002) 13(5): 985-95); L-tyrosine O-methyl, L-asparagine, L-glutamine, N-Boc-L-lysine (Akizawa et al., Bioconjugate Chem. (2013) 24:291-9); and glycyl-lysine (Arano et al., Cancer Res. (1999) 59:128-34); each of which is herein incorporated by reference in its entirety.

MMCM can be directly bound to the aromatic ring of Formula 1 (L₁=a direct bond) or can be bound to the aromatic ring through a linker (L₁). L₁ can be, e.g., a spacer such as a substituted or unsubstituted alkyl chain, a substituted or unsubstituted alkenyl chain, a substituted or unsubstituted alkynyl chain, or a short polyethylene glycol (PEG) chain (1-10 ethylene glycol units).

The positions of these three moieties (-L₁-MMCM, -L₂-CG, and -L₃-Y) on the aromatic ring of Formula 1 can vary. Where X is CH, these three moieties can be placed at any of the positions of the aromatic ring. In some such embodiments, the -L₂-CG, and -L₃-Y moieties are located at the 3 and 4 positions, respectively (or the 4 and 3 positions, respectively) relative to the -L₁-MMCM moiety (at the 1 position). In some such embodiments, the -L₂-CG, and -L₃-Y moieties are located at the 3 and 5 positions with respect to the -L₁-MMCM moiety, such that the aromatic ring comprises the referenced moieties at the 1, 3, and 5 positions. Where X is N, these three moieties can be placed at any of the remaining five positions of the ring, e.g., including, but not limited to, at the 2, 4, and 6 positions of the ring.

In particular embodiments, X is CH; L₃ is a bond (i.e., the radioactive halogen, Y, is connected directly to the phenyl ring); m is 1; L₂ is CH₂ and CG is Z-guanidine (such that L₂-CG is an alkyl guanidine, which can alternatively be described as L₂ is a bond and CG is an alkyl (e.g., methyl) guanidine); L₁ is a bond, and MMCM is a moiety (e.g., an NHS ester) suitable for reaction with an amine on the HER2-binding agent (forming an amide bond in the resulting targeted radiotherapeutic agent).

Certain prosthetic compounds within the scope of Formula 1 that can be complexed with HER2-binding agents to provide targeted radiotherapeutic agents as provided herein include compounds of Formula 1A and derivatives and variants thereof, as shown below. As shown, in Formula 1A, X is CH (i.e., the aromatic ring is a benzene ring), L₂ is a methylene group, and the three moieties (-L₁-MMCM, -L₃-Y, and —CH₂-CG) are present at the 1, 3, and 5 positions of the aromatic ring.

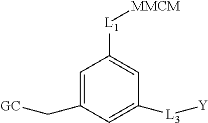

(Formula 1A: General Structure of Sub-Class IA Type Compounds)

Further, non-limiting prosthetic groups that can be coordinated to HER2-binding agents to give targeted radiotherapeutic agents encompassed by the present disclosure include, for example, those with the general structure of Formula 2 shown below (referred to as “Class II Type Compounds”).

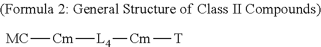

Certain such Class II type compounds are generally disclosed in Intl. Publ. No. WO2018/178936 (Duke University), which is incorporated herein by reference in its entirety. Such compounds include a polydentate metal chelating moiety (MC), a linker (L₄) with a conjugating moiety (Cm) at both ends of L₄, and a radiohalogenated template (T). T is a prosthetic compound and can be, for example, a compound of Formula 1 or a compound of Formula 1A, as shown above (a compound containing a MMCM). In some such embodiments, as referenced above, m=0, where the “MC-Cm-L₄-Cm” moiety of Formula 2 provides the desired function of the L₂-CG moiety in Formula 1, above (i.e., the MC-Cm-L₄-Cm substituent is a sufficiently “charged group”). In other such embodiments, m=1, 2, or 3, such that the aromatic ring of “T” has at least four substituents, i.e., L₁-MMCM, L₃-Y, L₂-CG, and Cm-L₄-Cm-MC, and may optionally comprise one or more additional L₂-CG substituents.

L₄ can be as defined above for L₁ and L₃. As such, L₄ can be a direct bond or can be, e.g., a spacer such as a substituted or unsubstituted alkyl chain, a substituted or unsubstituted alkenyl chain, a substituted or unsubstituted alkynyl chain, or a short polyethylene glycol (PEG) chain (1-10 ethylene glycol units). L₄ is again, as defined above but has NH, CO (carbonyl), or S (thioether) on one or both termini.

Cm can be, e.g., a thiourea, an amide, or a thioether. For example, in some embodiments, Cm is thiourea (e.g., when the conjugating functionality in the chelating moiety and T is an isothiocyanate), an amide (when the conjugating functionality in the chelating moiety and T is NHS or TFP active ester, or acyl halide), or thioether (when the conjugating functionality in the chelating moiety and T is maleimide).

T is generally a radiolabeled moiety containing a MMCM via which a macromolecule can be coupled to the compound to provide a targeted radiotherapeutic agent. As referenced above, T can, in some embodiments, be a compound/radical of Formula 1 or a compound/radical of Formula 1A. In other embodiments, other radiohalogen templates (T) can be used, including, but not limited to, iso-SGMIB, as disclosed in Choi et al., Nucl Med Biol. (2014) 41(10):802-12, SIPC, as disclosed in Reist et al., (1997) Nucl Med Biol. 24(7):639-48, which is incorporated herein by reference in its entirety; or SDMB, as disclosed in U.S. Pat. No. 5,302,700, which are all incorporated herein by reference in their entireties.

MC can be any polydentate moiety and can be cyclic or acyclic. The composition of MC can vary. MC can be either uncomplexed (lacking a metal) or complexed with the stable (nonradioactive) or radioactive form of a metal, e.g., a trivalent metal (M⁺³) such as lutetium, yttrium, indium, actinium, or gallium and the MC is connected to the linker either using one of the free COOH groups present on the MC or via other positions on the MC including one of the MC backbone carbons. Certain specific radioactive metals that can be complexed with the MC include, but are not limited to, radioactive metals selected from the group consisting of ¹⁷⁷Lu, ⁶⁴Cu, ¹¹¹In, ⁹⁰Y, ²²⁵Ac, ²¹³Bi, ²¹²Pb, ²¹²Bi, ⁶⁷Ga, ⁶⁸Ga, ⁸⁹Zr, and ²²⁷Th. It is noted that this list is not exhaustive and, although these exemplified radioactive metals are trivalent, certain MCs that may be used according to the present disclosure may bind metals of other valences, and such MCs and radioactive metals are also encompassed herein.

In some embodiments, the inclusion of a radioactive metal associated with the MC can eliminate the need for a radioactive atom elsewhere on the molecule (e.g., as “Y” when T of Formula 2=a moiety of Formula 1/1a). As such, in Formula 2 compounds, “T” may or may not include a radioactive atom (e.g., halogen). In some embodiments, T comprises a moiety as shown in Formula 1/1a above, wherein the “Y” group is a non-radioactive halogen (e.g., a non-radioactive bromine or iodine). In other embodiments, a compound of Formula 2 is provided which comprises both a radiohalogen (e.g., as “Y” when T of Formula 2=a moiety of Formula 1/1a) and a radiometal (associated with MC, such as the radioactive metals referenced above). In certain particular embodiments, such a strategy would allow, e.g., for use of the same prosthetic agent for multiple isotopes. In certain specific examples, a compound of Formula 2 is provided with a low energy beta emitter (e.g., ¹³¹I) plus a high energy beta emitter (e.g., ⁹⁰Y); or an alpha emitter (e.g., ²²⁵Ac) metal and a beta emitter halogen (e.g., ¹³¹I); or an alpha emitter halogen (e.g., ²¹¹At) and a beta emitter radiometal (e.g., ¹⁷⁷Lu).

In some embodiments, MC is a macrocyclic ligand, consisting of a ring containing 8 or more atoms, bearing at least 3 negatively charged substituents such as carboxyl or phosphonate groups. Exemplary macrocyclic ligands suitable as the MC group include 1,4,7,10-tetraazacyclododecane-1,4,7,10-tetraacetic acid (DOTA), 1,4,7-triazacyclononane-1,4,7-triacetic acid (NOTA), 1,4,8,11-tetraazacyclotetradecane-1,4,8,11-tetraacetic acid (TETA), and 1,4,7-triazacyclononane-1,4,7-tri(methylene phosphonic acid) (NOTP). In other embodiments, MC is MeO-DOTA, as disclosed in Gali et al., Anticancer Research (2001) 21(4A):2785-92, which is incorporated herein by reference in its entirety.

An example of a Class II compound suitable for conjugation to a HER2-binding agent to form a targeted radiotherapeutic agent according to the present disclosure is illustrated below in Formula 2A, wherein MC is a macrocyclic ligand comprising DOTA, and wherein the radiohalogenated template T is a moiety corresponding to Formula 1.

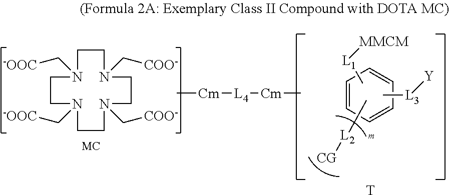

The left-hand brackets in Formula 2A are intended to convey that the specific site on the MC (DOTA) to which the Cm group is bonded is not limited, i.e., the Cm may be bonded to DOTA at various sites thereon. Similarly, the right-hand brackets in Formula 2A are intended to convey that the specific site on the ring of “T” to which the Cm group is bonded is not limited, i.e., Cm may be bonded to T at various sites on the ring. Again, as referenced above, CG-L₂ may or may not be present. In some embodiments, the benzene ring of T in Formula 2A comprises four substituents (including the linked MC, L₂-MMCM, L₃-Y, and L₂-CG). In other embodiments, the benzene ring of T in Formula 2A comprises three substituents (including the linked MC, L₂-MMCM, and L₃-Y). The latter embodiments are particularly relevant when the linked MC is charged, i.e., it can take the place in providing the desired function of the “L₂-CG” substituent.

In some embodiments, MC is an acyclic ligand, consisting of a chain containing 6 or more atoms bearing at least 3 negatively charged substituents such as carboxyl or phosphonate groups. Exemplary acyclic ligands suitable as the MC group include diethylenetriaminepentaacetic acid (DTPA), ethylenediaminetetramethylenephosphonic acid (EDTMP), and ethylenediaminetetraacetic acid (EDTA). An example of a Class II compound is illustrated below in Formula 2B, wherein MC is an acyclic ligand comprising DTPA, and wherein the radiohalogenated template T is a moiety corresponding to Formula 1.

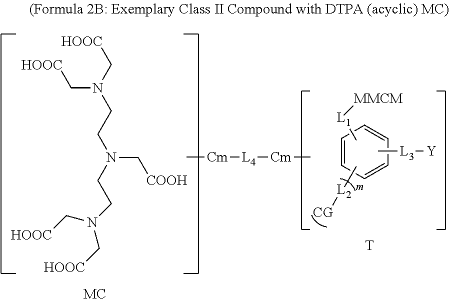

As referenced above with respect to Formula 2A, the left-hand brackets in Formula 2B are intended to convey that the specific site on the MC (DTPA) to which the Cm group is bonded is not limited, i.e., the Cm may be bonded to DTPA at various sites thereon. Similarly, the right-hand brackets in Formula 2B are intended to convey that the specific site on the ring of “T” to which the Cm group is bonded is not limited, i.e., Cm may be bonded to T at various sites on the ring. Again, as referenced above, CG-L₂ may or may not be present. In some embodiments, the benzene ring of T in Formula 2B comprises four substituents (including the linked MC, L₂-MMCM, L₃-Y, and L₂-CG). In other embodiments, the benzene ring of T in Formula 2A comprises three substituents (including the linked MC, L₂-MMCM, and L₃-Y). The latter embodiments are particularly relevant when the linked MC is charged, i.e., it can take the place in providing the desired function of the “L₂-CG” substituent.

In some specific embodiments, a targeted radiotherapeutic agent is provided by attaching a HER2-binding agent as described herein to a compound of Formula 2 wherein MC=DOTA, L₄=—NH(CH₂)₆NH—, T=3-iodo-5-succinimidyloxycarbonyl-benzoyl, Cm=amide and MMCM=N-hydroxysuccinimide ester, a maleimide-containing moiety, or (Gly) n for site-specific conjugation using sortase (refer to the Formulas above).

Further, non-limiting prosthetic groups that can be coordinated to HER2-binding agents to give targeted radiotherapeutic agents encompassed by the present disclosure include, for example, those with the general structure of Formula 3 shown below (referred to as “Class III Type Compounds”).

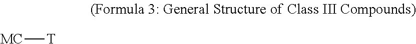

Certain such Class IIII type compounds are generally disclosed in WO 2021/096968 (Duke University), which is incorporated herein by reference in its entirety. In such compounds, MC can, in some embodiments, be as defined above (and can thus be cyclic or acyclic and can be uncomplexed or complexed with a metal). In some embodiments, the disclosed prosthetic agents and corresponding targeted radiotherapeutic agents comprise a functionalized MC, e.g., a modified DOTA or a modified DOTA. One exemplary modified DOTA is DOTA-tris(t-Bu ester), i.e., DOTA comprising tert-butyl acetate groups associated with each nitrogen atom other than the nitrogen atom when it is used to connect to “T.” Such a compound, suitable for reaction to provide MC-T is also referred to as tri-tert-butyl 1,4,7,10-tetraazacyclododecane-1,4,7-tricarboxylate. Upon removal of the tert-butyl protecting groups, a deprotected form is provided, which can be referred to as 1,4,7,10-tetraazacyclododecane-1,4,7-tricarboxylic acid. A further modified DOTA that may find use in certain embodiments of the disclosure is an acyclic analogue, e.g., an acyclic analogue of DOTA or an acyclic analogue of a modified DOTA. One exemplary tert-butyl acetate-functionalized acyclic DOTA that can be reacted to form MC-T is tert-butyl (2-((2-((2-aminoethyl)(tert-butoxycarbonyl)-amino)ethyl)(tert-butoxycarbonyl)amino)ethyl)glycinate. Upon removal of the tert-butyl protecting groups, a deprotected form is provided, which can be referred to as (2-((2-((2-aminoethyl)(carboxy)amino)ethyl)(carboxy)amino)ethyl)glycine. Similarly, in some embodiments, a modified analogue of NOTA is employed, which comprises tert-butyl acetate groups associated with each nitrogen atom other than the nitrogen atom when it is used to connect to “T.” Such a modified NOTA can be di-tert-butyl 1,4,7-triazonane-1,4-dicarboxylate, which can be reacted via the nitrogen atom without a tert-butyl group attached thereto to give MC-T. It is understood that, in all embodiments herein providing a tert-butyl acetate group associated with the MC group, the deprotected form (comprising an acetic acid group in place of the tert-butyl acetate group) is also intended to be encompassed herein.

Certain such modified “MC” groups are shown below in Formulas A-1, A-2, and A-3, respectively, with one feasible point of attachment to “T” shown on each depicted “MC” moiety, providing relevant radiohalogen prosthetic moieties, which can be attached to a HER2-binding agent to provide targeted radiotherapeutic agents comprising the same.

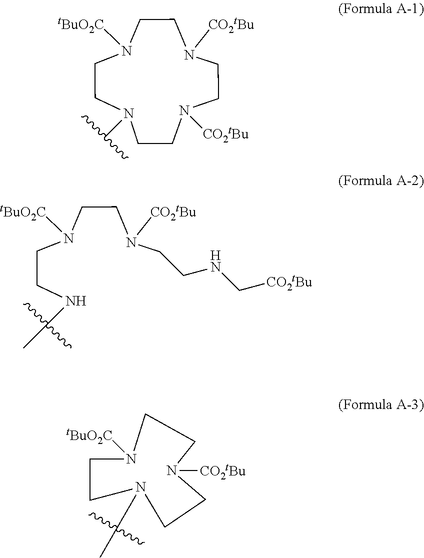 [SMILES: *N1CCN(C(=O)OC(C)(C)C)CCN(C(=O)OC(C)(C)C)CCN(C(=O)OC(C)(C)C)CC1 ; *NCCN(CCN(CCNCC(=O)OC(C)(C)C)C(=O)OC(C)(C)C)C(=O)OC(C)(C)C ; *N1CCN(C(=O)OC(C)(C)C)CCN(C(=O)OC(C)(C)C)CC1]

In such embodiments, MC is connected to the remainder of the prosthetic moiety (comprising “T”) via any relevant atom, e.g., via a carbon or nitrogen associated with the MC (although not limited thereto) to give corresponding radiohalogen prosthetic moieties. For the modified DOTA and NOTA groups referenced herein, MC can be advantageously connected to T via a nitrogen present on the modified DOTA/NOTA (as shown in Formulas A-1, A-2, and A-3 above) or via other positions on the MC (including, e.g., via one of the MC backbone carbons).

T is a radiohalogenated template, as referenced in detail above. In certain embodiments, T can be a compound of Formula 1 or a compound of Formula 1A. In some embodiments, T is a radiohalogenated template of Formula B, below, where its connection to MC is shown by the wavy line. T can be directly bonded to the MC, i.e., with a direct bond between the carbon atom shown adjacent to R₁ and a moiety on MC (e.g., where MC is DOTA/NOTA or a modified DOTA/NOTA) or the carbon atom shown below can be directly bonded to a nitrogen atom of the backbone DOTA/NOTA structure.

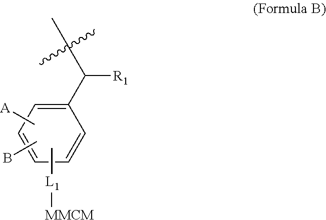

In Formula B, the following definitions are applicable:
R₁=H, an ester, or a carboxylic acid.
A is R₂-R₃—Y or Y.
B is H, an alkoxy (e.g., methoxy, ethoxy, propoxy, or butoxy) group, or a halogen (e.g., a non-radioactive halogen, such as selected from the group consisting of Cl, F, I, and Br).
R₂=a direct bond, an alkyl group, or an oxygen-containing moiety. Certain examples of “oxygen-containing moieties” as provided herein include, but are not limited to: —O—, —O—CH₂—, —O—CH₂CH₂—, and the like.

R₃=a direct bond, an alkyl group, or an aromatic moiety (e.g., a phenyl ring).

Other definitions are as presented above with respect to compounds of other formulas.

In certain embodiments, R₂ is —O—CH₂— and R₃ is a phenyl ring, such that R₂-R₃ is a benzyloxy group attached to the central phenyl ring of Formula C. The positions of the substituents on the phenyl ring relative to one another can vary. L₁-MMCM on the aromatic ring relative to the other substituents (A, B, and the connection to MC) can vary. In certain embodiments, the L₁-MMCM group is meta to the connection to MC. The central phenyl ring of the compound, in some embodiments, comprises no substituents ortho to L₁-MMCM (such that the carbon to which L₁-MMCM is connected is ortho to two non-substituted carbon atoms). In particular embodiments, A is ortho to the carbon connected to MC. Where A is ortho to the carbon connected to MC, in some such embodiments, B is ortho to the A substituent, and is H. In particular embodiments, A is meta to the carbon connected to MC. Where A is meta to the carbon connected to MC, in some such embodiments, B is ortho to the carbon connected to MC (and meta to the A substituent), and in certain embodiments, B is alkoxy (e.g., methoxy).

Various embodiments within the scope of Formula B are shown below. For example, in some embodiments, R₁ is an ester group, e.g., CO₂^{t}Bu, e.g., as shown in Formula B-1, below. In some embodiments, the central aromatic ring of T is functionalized with an alkoxy group as substituent B and, in particular, a methoxy (OCH₃) group, e.g., with three other substituents on the aromatic ring, e.g., as shown in Formula B-2 below. In some embodiments, the halogenation site or precursor moiety is placed on an aromatic ring that is attached (directly or indirectly) to the central aryl ring (e.g., including, but not limited to, being connected to the central aryl ring via an O-alkyl- moiety), as shown in Formula B-3 where, although the location of the halogen on the phenyl ring can vary, the halogen is para to the connection to the remainder of the molecule. In some such embodiments as shown in Formula C-3, B is H. In certain embodiments, as shown in Formula B-4, the halogen site is on an aromatic ring connected to the central aryl ring, where the aromatic ring is directly bonded to the central aryl ring, as shown. In some such embodiments as shown in Formula B-4, B is H. It is noted that, in embodiments wherein the halogenation site or precursor moiety is on an aromatic ring attached to the central aryl ring, the aromatic ring may comprise no other substituents, or in some embodiments, may comprise one or more additional substituents at varying positions with respect to the halogenation site or precursor moiety. For example, in some embodiments, the aromatic ring further comprises a guanidinomethyl. Thus, although, e.g., Formulas B-3 and B-4 below are shown with an aromatic ring substituted only with I (which can alternatively be any “Y” group provided herein), the disclosure is not limited thereto, and other substituents may be present on that ring in various embodiments. In particular embodiments, a moiety of Formula C-3 is provided wherein the aryl ring is substituted with both I (or other Y) and a guanidinomethyl group.

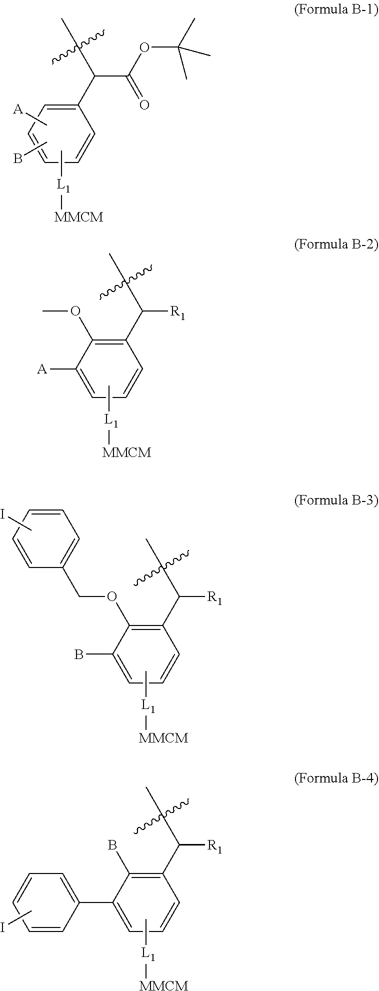

In some embodiments, the halogenation site/precursor moiety is directly on the central aryl ring (e.g., connected via a direct bond), as explicitly shown in Formulas B-5 and B-6. In some such embodiments as shown in Formula B-5, B is H. In Formula B-5, the iodination site is proximal the chelation. In some such embodiments as shown in Formula B3-6, B is advantageously OCH₃.

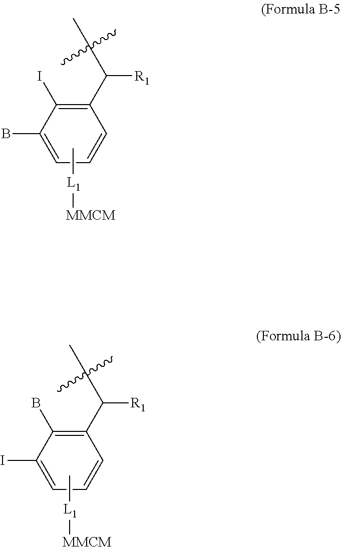

In specific embodiments, DOTA-SIB or NOTA-SIB hybrid radiohalogen prosthetic moieties, shown below, are attached to a HER2-binding agent as described herein to provide a targeted radiotherapeutic agent. Although “I” is shown as the radiohalogen in the structures, it is to be understood that various “Y” groups as provided herein may be substituted at that position; further, the N-hydroxysuccinimidyl ester shown can be replaced with other groups (e.g., a TFP ester group) for attachment to the HER2-binding agent. Formulas 3A, 3B, 3B′, 3C, and 3D depict non-limiting prosthetic moieties with DOTA-SIBs (with 3B′ providing a guanidinomethyl group on the aromatic ring (which can be ortho or meta to the I substituent), and where X and Y can be placed in any of the five positions on the aromatic ring; if X is an iodine radioisotope or another radiohalogen (or a precursor thereto), Y is guanidinomethyl, and vice versa). Formula 3E depicts a non-limiting prosthetic moiety with a NOTA-SIB respectively,

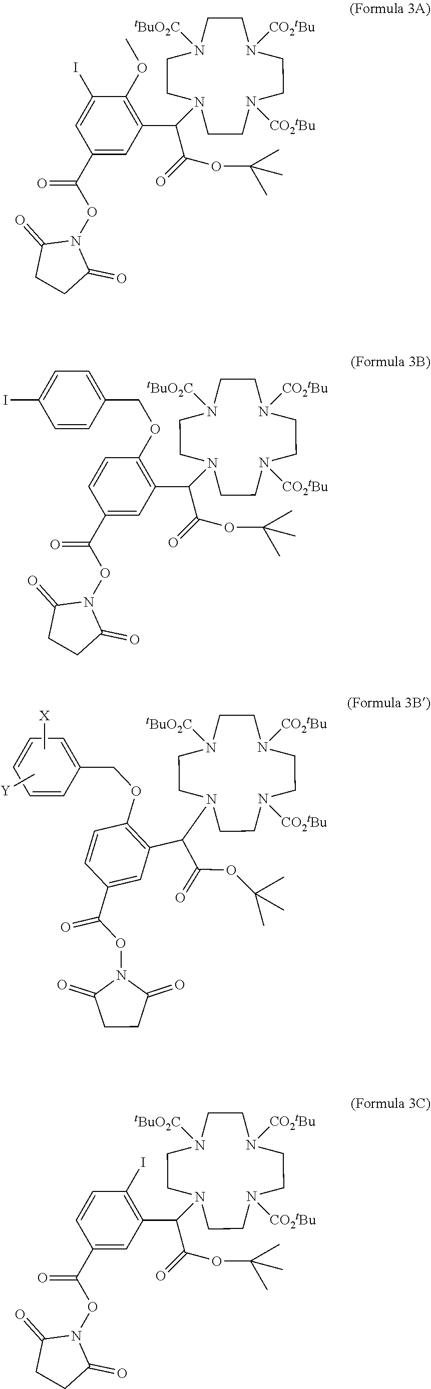

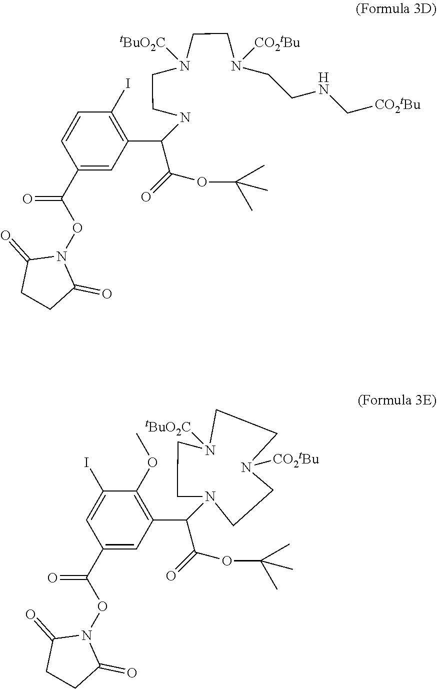 [SMILES: CC(C)(C)OC(=O)CNCCN(CCN(CCNC(C(=O)OC(C)(C)C)c1cc(C(=O)ON2C(=O)CCC2=O)ccc1I)C(=O)OC(C)(C)C)C(=O)OC(C)(C)C ; COc1c(I)cc(C(=O)ON2C(=O)CCC2=O)cc1C(C(=O)OC(C)(C)C)N1CCN(C(=O)OC(C)(C)C)CCN(C(=O)OC(C)(C)C)CC1]

Non-limiting examples of structures wherein L₁-MC is a tetrafluorophenol (TFP) ester are shown in Formulas 3F to 3H below, and these can be further modified as described above (e.g., including, but not limited to, replacement of the I with other Y groups, and addition of one or more substituents on the O-alkyl phenyl group of Formula 3G or 3H).

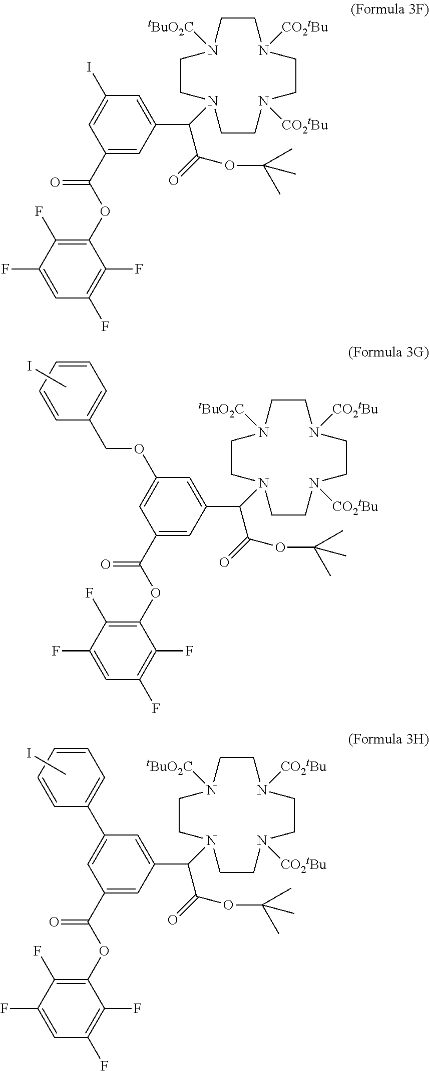

Although, in most structures within Formula 3 shown comprise an ester-containing R₁-substituent (e.g., a CO₂^{t}Bu group), in other embodiments, a radiohalogen prosthetic moiety or precursor thereof is provided wherein R₁ is H, as shown in Formula 3I, below.

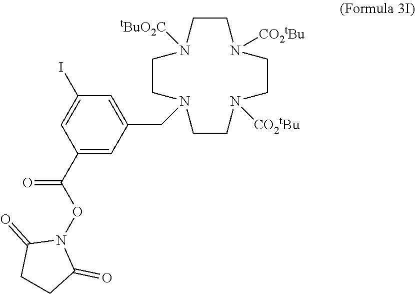 [SMILES: CC(C)(C)OC(=O)N1CCN(Cc2cc(I)cc(C(=O)ON3C(=O)CCC3=O)c2)CCN(C(=O)OC(C)(C)C)CCN(C(=O)OC(C)(C)C)CC1]

Additional, non-limiting radiohalogen prosthetic moieties that can be attached to HER2-binding agents to give targeted radiotherapeutic agents encompassed by the present disclosure include, for example, N-succinimidyl 5-[*I]iodo-3-pyridinecarboxyl ([*I]SIPC); SFPC, SAPC, or any other radiohalogen derivatives; N-succinimidyl-3-[*I]-iodobenzoate ([*I]SIB) and SFB, SAB or other halogen radionuclides; N-succinimidyl-4-guanidinomethyl-3-[*I]iodobenzoate ([*I]SGMIB), as well as SAGMB, SFGMB, or other radiohalogen derivatives; N-maleimidoethyl-3-(guanidinomethyl)-4-[*I]-iodobenzamide ([*I]MEGMIB); compounds comprising an acyclic bifunctional chelator, including, for example, p-SCN-Bz-DTPA; tetra-t-Bu-DTPA; mercaptoacetyltriglycine (MAG3); MX-DTPA; CHX-DTPA (where CHX can be CHX-A or CHX-B), or a macrocyclic bifunctional chelator, including P-SCN-Bn-DOTA; maleimidocysteineamido-DOTA derivatives; and benzyl TETA derivatives.

### Definitions

“Cₘ-Cₙalkyl” on its own or in composite expressions such as Cₘ-Cₙhaloalkyl, Cₘ-Cₙalkylcarbonyl, Cₘ-Cₙalkylamine, etc., represents a straight or branched aliphatic hydrocarbon radical having the number of carbon atoms designated, e.g., C₁-C₄alkyl means an alkyl radical having from 1 to 4 carbon atoms. C₁-C₆alkyl has a corresponding meaning, including also all straight and branched chain isomers of pentyl and hexyl. Exemplary alkyl radicals for use in the preparing the provided compositions are C₁-C₆alkyl, including methyl, ethyl, n-propyl, isopropyl, n-butyl, isobutyl, sec-butyl, tert-butyl, n-pentyl and n-hexyl, especially C₁-C₄alkyl such as methyl, ethyl, n-propyl, isopropyl, t-butyl, n-butyl and isobutyl. In particular embodiments, the alkyl radical(s) are methyl and/or isopropyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(═O)-alkyl, —O—C(═O)-aryl, —O—C(═O)-cycloalkyl, —C(═O)OH and —C(═O)O-alkyl. In particular embodiments, the alkyl group is unsubstituted, unless otherwise indicated.

“C₂-Cₙalkenyl” represents a straight or branched aliphatic hydrocarbon radical containing at least one carbon-carbon double bond and having the number of carbon atoms designated, e.g., C₂-C₄alkenyl means an alkenyl radical having from 2 to 4 carbon atoms; C₂-C₆alkenyl means an alkenyl radical having from 2 to 6 carbon atoms. Non-limiting alkenyl groups include ethenyl, propenyl, n-butenyl, 3-methylbut-2-enyl, n-pentenyl and hexenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(═O)-alkyl, —O—C(═O)-aryl, —O—C(═O)-cycloalkyl, —C(═O)OH and —C(═O)O-alkyl. It particular embodiments, the alkenyl group is unsubstituted, unless otherwise indicated.

“C₂-Cₙalkynyl” represents a straight or branched aliphatic hydrocarbon radical containing at least one carbon-carbon triple bond and having the number of carbon atoms designated, e.g., C₂-C₄alkynyl means an alkynyl radical having from 2 to 4 carbon atoms; C₂-C₆alkynyl means an alkynyl radical having from 2 to 6 carbon atoms. Non-limiting alkenyl groups include ethynyl, propynyl, 2-butynyl and 3-methylbutynyl pentynyl and hexynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In particular embodiments, the alkynyl group is unsubstituted, unless otherwise indicated.

The term “Cₘ-Cₙhaloalkyl” as used herein represents Cₘ-Cₙalkyl wherein at least one C atom is substituted with a halogen (e.g., the Cₘ-Cₙhaloalkyl group may contain one to three halogen atoms), e.g., iodine, bromine, or fluorine. Typical haloalkyl groups are C₁-C₂haloalkyl, in which halo suitably represents iodo. Exemplary haloalkyl groups include iodomethyl, diiodomethyl and triiodomethyl. As used herein, only one of the halogens can be radioactive.

The term “Cₘ-Cₙhydroxyalkyl” as used herein represents Cₘ-Cₙalkyl wherein at least one C atom is substituted with one hydroxy group. Typical Cₘ-Cₙhydroxyalkyl groups are Cₘ-Cₙalkyl wherein one C atom is substituted with one hydroxy group. Exemplary hydroxyalkyl groups include hydroxymethyl and hydroxyethyl.

The term “Cₘ-Cₙalkylene” as used herein represents a straight or branched bivalent alkyl radical having the number of carbon atoms indicated. In particular embodiments, Cₘ-Cₙalkylene radicals for used in preparing the provided compositions are C₁-C₃alkylene. Non-limiting examples of alkylene groups include —CH₂—, —CH₂CH₂—, —CH₂CH₂CH₂—, —CH(CH₃)CH₂CH₂—, —CH(CH₃)— and —CH(CH(CH₃)₂)—.

“Cₘ-Cₙalkoxy” represents a radical Cₘ-Cₙalkyl-O— wherein Cₘ-Cₙalkyl is as defined above. In particular embodiments, Cₘ-Cₙalkoxy is C₁-C₄alkoxy which includes methoxy, ethoxy, n-propoxy, isopropoxy, t-butoxy, n-butoxy, sec-butoxy and isobutoxy. In particular embodiments, the Cₘ-Cₙalkoxy is methoxy or isopropoxy. C₁-C₆alkoxy has a corresponding meaning, expanded to include all straight and branched chain isomers of pentoxy and hexoxy.

The term “Me” means methyl, and “MeO” means methoxy. The term “amino” represents the radical —NH₂. The term “halo” represents a halogen radical such as fluoro, chloro, bromo, iodo, or astato. Typically, halo groups are iodo, bromo or astato. The term “aryl” represents an aromatic ring, for example a phenyl, biphenyl or naphthyl group.

The term “heterocycloalkyl” represents a stable saturated monocyclic 3-12 membered ring containing 1-4 heteroatoms independently selected from O, S and N. In some embodiments, the stable saturated monocyclic 3-12 membered ring contains 4 N heteroatoms. In a second embodiment the stable saturated monocyclic 3-12 membered ring contains 2 heteroatoms independently selected from O, S and N. In a third embodiment the stable saturated monocyclic 3-12 membered ring contains 3 heteroatoms independently selected from O, S and N. A heterocycloalkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O— alkyl. In particular embodiments, the heterocycloalkyl group is unsubstituted, unless otherwise indicated.

The term “heteroaryl” represents a stable aromatic ring containing 1-4 heteroatoms independently selected from O, S and N. In particular embodiments, heteroaryl moieties useful in the present disclosure have 6 ring atoms. In some embodiments, the stable aromatic ring system contains one heteroatom that is N.

The term “aminoCₘ-Cₙalkyl” represents a Cₘ-Cₙalkyl radical as defined above which is substituted with an amino group, i.e., one hydrogen atom of the alkyl moiety is replaced by an NH₂-group. Typically, “aminoCₘ-Cₙalkyl” is aminoC₁-C₆alkyl.

The term “aminoCₘ-Cₙalkylcarbonyl” represents a Cₘ-Cₙalkylcarbonyl radical as defined above, wherein one hydrogen atom of the alkyl moiety is replaced by an NH₂-group. Typically, “aminoCₘ-Cₙalkylcarbonyl” is aminoC₁-C₆alkylcarbonyl. Examples of aminoCₘ-Cₙalkylcarbonyl include but are not limited to glycyl: C(═O)CH₂NH₂, alanyl: C(═O)CH(NH₂)CH₃, valinyl: C═OCH(NH₂)CH(CH₃)₂, leucinyl: C(═O)CH(NH₂)(CH₂)₃CH₃, isoleucinyl: C(═O)CH(NH₂)CH(CH₃)(CH₂CH₃) and norleucinyl: C(═O)CH(NH₂)(CH₂)₃CH₃ and the like. This definition is not limited to naturally occurring amino acids.

Related terms are to be interpreted accordingly in line with the definitions provided above and the common usage in the technical field.

As used herein, the term “(═O)” forms a carbonyl moiety when attached to a carbon atom. It should be noted that an atom can only carry an oxo group when the valency of that atom so permits.

The term “monophosphate, diphosphate and triphosphate ester” refers to groups:

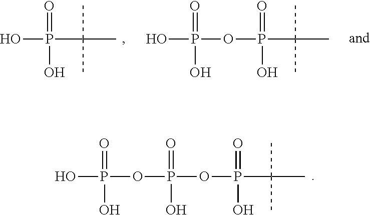

The term “thio-monophosphate, thio-diphosphate and thio-triphosphate ester” refers to groups:

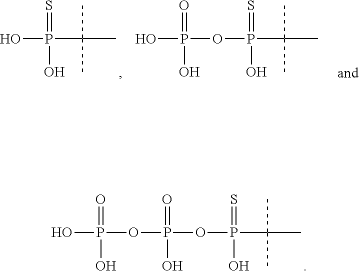

As used herein, the radical positions on any molecular moiety used in the definitions may be anywhere on such a moiety as long as it is chemically stable. When any variable present occurs more than once in any moiety, each definition is independent.

Whenever used herein, the phrases “compounds of Formula 1”, “compounds of Formula 1A,” “compounds of Formula 2,” “compounds of Formula 3,” or “the compounds of the disclosure” or similar phrases, are meant to include the compounds of Formula 1 and subgroups of compounds of Formula 1, the compounds of Formula 2 and subgroups of compounds of Formula 2, and the compounds of Formula 3 and subgroups of Formula 3, respectively, including the possible stereochemically isomeric forms, and their pharmaceutically acceptable salts and solvates.

The term “solvates” covers any pharmaceutically acceptable solvates that the compounds of Formula 1, 2, and 3 as well as the salts thereof, are able to form. Such solvates are, for example, hydrates, alcoholates, e.g., ethanolates, propanolates, and the like, especially hydrates.

In general, the names of compounds provided herein are generated using ChemDraw Professional 16.0. In addition, if the stereochemistry of a structure or a portion of a structure is not indicated with for example bold or dashed lines, the structure or portion of that structure is to be interpreted as encompassing all stereoisomers of it.

Linkers may also be selected to facilitate bonding of the respective moieties to the core structure. For example, as discussed in greater details below with respect to an exemplary synthesis pathway for the prosthetic compound, a representative linker is a bifunctional alkyl chain (e.g., —CH₂—, —C₂H₄—, —C₃H₆—, etc.) having from 1 to 6 carbon atoms, in which one carbon atom may be substituted with a cyclic (hydrocarbon ring) radical or heterocyclic (heterocyclic ring) radical. Representative heterocyclic radicals have at least one nitrogen atom in the heterocyclic ring. Specific examples of such heterocyclic radicals are therefore diazinyl, diazolyl, triazinyl, triazolyl, tetrazinyl, and tetrazolyl radicals. These and other heterocyclic radicals, or otherwise cyclic radicals, may optionally be fused to a another cyclic or heterocyclic radical, or otherwise fused to a another cyclic or heterocyclic radical that is itself part of a fused ring system (e.g., a triazolyl radical may be fused to an 8-membered cyclic or heterocyclic radical that is itself fused to two 6-membered cyclic rings, as in the case of the triazolyl radical (or other nitrogen atom-substituted heterocyclic hydrocarbon radical) being fused to a dibenzoazocanyl radical). Therefore, linkers containing three or more fused rings, such as hydrocarbon rings, heterocyclic rings, and combinations of these rings, are possible. A representative charged group linker, L₂, is a bivalent substituted or unsubstituted alkyl chain having from 1 to 6 carbon atoms, a substituted or unsubstituted alkenyl chain, or a substituted or unsubstituted alkynyl chain. Generally, L₁, L₂, L₃ and/or L₄ may be (or may comprise) substituted or unsubstituted bivalent alkyl radicals, having from 1 to 6 carbon atoms, wherein one or more carbon atoms may be substituted with and/or replaced by a heteroatom such as NH, O, or S, or otherwise may be substituted with or replaced by another alkyl radical (e.g., resulting in the formation of a branched alkyl radical) having from 1 to 8 carbon atoms that may be linear, branched, or cyclic. For example, one carbon atom of an alkyl radical may be substituted to provide a carbonyl (C═O) group, and an adjacent carbon atom replaced by NH, thereby resulting in a peptide/amide linkage (C═O)—NH—. Representative linkers L₁, L₂, L₃, and L₄ can therefore include divalent alkyl radicals having one or more of such peptide linkages, NH linkages, (C═O) linkages, and/or cyclic —C₆H₄— linkages, including combinations of any two, three, or four of such linkages, incorporated into the alkyl chain. In addition, in the case of bivalent alkyl radicals for L₁, L₂, and/or L₃, a carbon-carbon double bond and/or a carbon-carbon triple bond may be formed between one or more pairs of adjacent carbon atoms, to provide bivalent, unsaturated (e.g., olefinic) alkyl radicals.

In some embodiments, the HER2-binding agents are targeted radiotherapeutic agents having the structure:

VHH-RLC,

wherein VHH is a protein comprising a HER2-binding VHH domain provided herein (e.g., having a sequence of any one of SEQ ID NOs: 1-81 and 205, or a fragment or variant thereof), and RLC, is a radiolabeling chemistry used to facilitate attachment of a radionuclide to the VHH, either directly or indirectly. In some embodiments, RLC is a Class I compound, Class II compound, Class III compound, compound, an indirect halogenation prosthetic compound, an Acyclic Bifunctional chelator.

In some embodiments, RLC is a Class I compound. Examples of certain, non-limiting targeted radiotherapeutic agents falling within the scope of the disclosure are as follows, wherein VHH is a protein comprising a HER2-binding VHH domain provided herein (e.g., having a sequence of any one of SEQ ID NOs: 1-81 and 205, or a fragment or variant thereof):

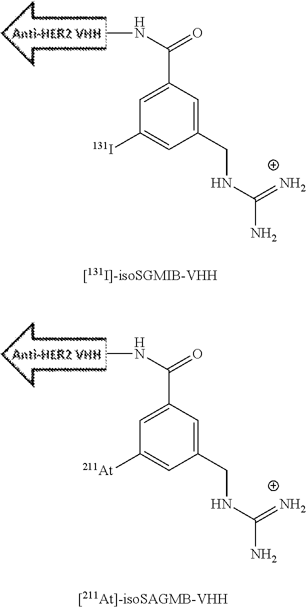

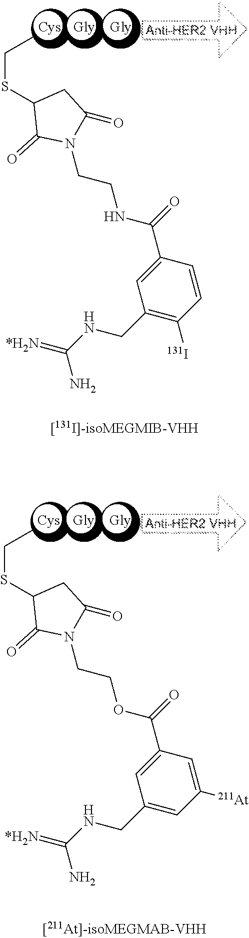

In some embodiments, RLC is

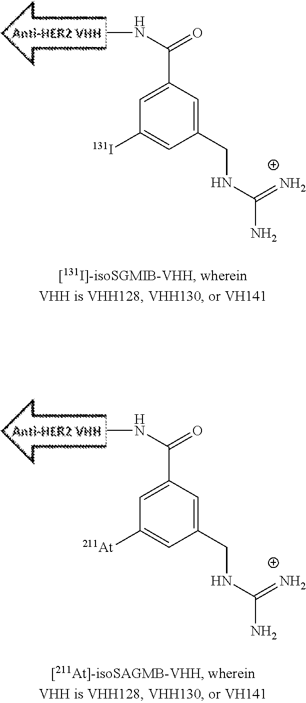

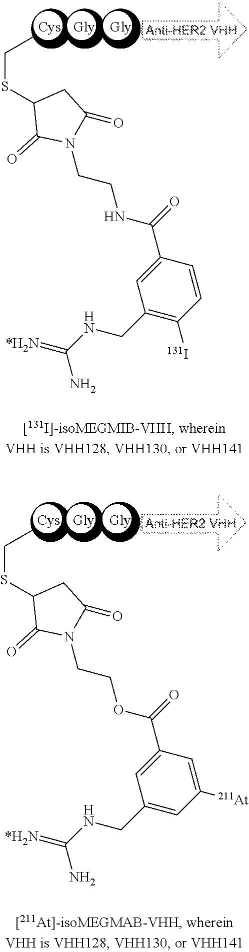

In some embodiments, RLC is a Class II compound. Examples of certain, non-limiting targeted radiotherapeutic agents falling within the scope of the disclosure are as follows, wherein VHH is a protein comprising a HER2-binding VHH domain provided herein (e.g., having a sequence of any one of SEQ ID NOs: 1-81 and 205, or a fragment or variant thereof):

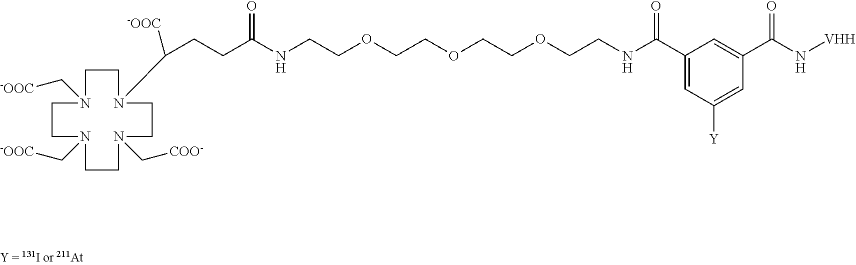

In some embodiments, RLC is a Class III compound. Examples of certain, non-limiting targeted radiotherapeutic agents falling within the scope of the disclosure are as follows: wherein VHH is a protein comprising a HER2-binding VHH domain provided herein (e.g., having a sequence of any one of SEQ ID NOs: 1-81 and 205, or a fragment or variant thereof),

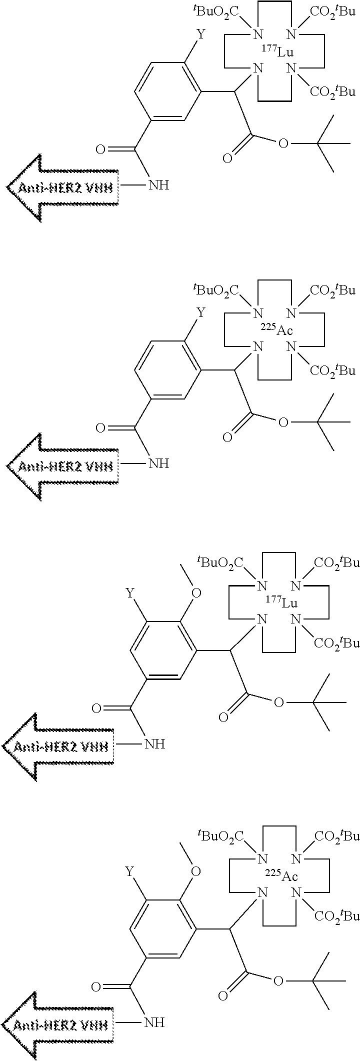

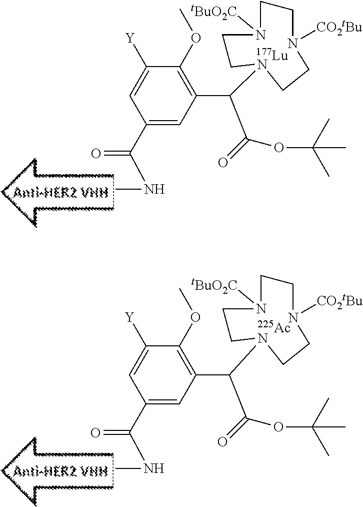

In some embodiments, RLC is a direct radiolabeling using electrophilic substitution reactions for radiohalogenation. In some embodiments, RLC is a direct radiolabeling using electrophilic substitution reactions using an Iodogen, chloragen T, and N-halosuccinimides for radiohalogenation.

In some embodiments, RLC is an indirect halogenation prosthetic compound. In some embodiments, RLC is N-succinimidyl 5-[*I]iodo-3-pyridinecarboxyl ([*I]SIPC). In some embodiments, RLC is SFPC, SAPC, or any other radiohalogen derivative known in the art. In some embodiments, RLC is N-succinimidyl-3-[*I]-iodobenzoate ([*I]SIB). In some embodiments, RLC is SFB, SAB or any other halogen radionuclide known in the art. In some embodiments, RLC is N-succinimidyl-4-guanidinomethyl-3-[*I]iodobenzbate ([*I]SGMIB). In some embodiments, RLC is SAGMB, SFGMB, or any other radiohalogen derivative known in the art. In some embodiments, RLC is N-maleimidoethyl-3-(guanidinomethyl)-4-[*I]-iodobenzamide ([*I]MEGMIB).

In some embodiments, RLC is an Acyclic Bifunctional chelator. In some embodiments, RLC is p-SCN-Bz-DTPA, tetra-t-Bu-DTPA, mercaptoacetyltriglycine (MAG3), or MX-DTPA. In some embodiments, RLC is CHX-DTPA (where CHX can be CHX-A or CHX-B).

In some embodiments, RLC is an Acyclic Bifunctional chelator. In some embodiments, RLC is P-SCN-Bn-DOTA. In some embodiments, RLC is an maleimidocysteineamido-DOTA derivative. In some embodiments, RLC is a benzyl TETA derivative.

Radiohalogen prosthetic moieties and corresponding targeted radiotherapeutic agents can be prepared in various ways, e.g., according to known methods in the art. Targeted radiotherapeutic agents can be provided, e.g., by attaching a radionuclide/radiohalogen prosthetic moiety to a HER2-binding agent as provided herein, either directly or indirectly. Certain such methods are described, for example, in Intl. Publ. Nos. WO2018/178936 and WO2021/096968 (Duke University), each of which is incorporated herein by reference in its entirety.

For example, in some embodiments, a precursor to a radiohalogen prosthetic moiety is prepared (e.g., comprising a tin or other alkyl metal moiety, or a boronic acid moiety or boronic ester moiety, such as wherein Y in the formulas above is, e.g., a tin or other alkyl metal moiety, or a boronic acid moiety or boronic ester moiety). The precursor can then be radiolabeled and purified. Such radiolabeling approaches may employ, in some embodiments, electrophilic substitution reactions and can be performed using one or more catalysts, e.g., including, but not limited to, 1,2,3,6-tetrachloro-3α,6α-diphenylglycouril (Iodo-gen®), Chloramine-T, or an N-halosuccinimide. The resulting radiohalogen prosthetic moiety is then conjugated with a HER2-binding agent using standard conditions to give the targeted radiotherapeutic agent. Non-limiting examples of the preparation of certain radioiodinated prosthetic agents and use of such radioiodinated prosthetic agents to label HER2-binding agents are provided, e.g., in Vaidyanathan et al., Nat Prot. (2007) 2:282-6; and Choi et al., Nucl Med Biol. (2014) 1:802-12, which are incorporated herein by reference in their entireties; a non-limiting example of the preparation of ²¹¹At-labeled prosthetic agents and use of such agents to label HER2-binding agents is provided in Intl. Publ. No. WO2018/178936, which is incorporated herein by reference in its entirety.

In some embodiments, a “preconjugation” approach is employed. For example, a precursor to a radiohalogen prosthetic moiety is first prepared and reacted with the HER2-binding agent and the HER2-binding agent-precursor conjugate is then radiolabeled to give the targeted radiotherapeutic agent. In some such embodiments, one drawback (particularly for radioiodination) is that constituent tyrosine, phenylalanine, and even histidine residues that may be present on the HER2-binding agent also can get radioiodinated in addition to the intended prosthetic site(s) for radiolabeling (i.e., moieties bearing the alkyl metal group or boronic acid or boronic ester group). The problem with the radiohalogens on the tyrosines is that the radioactivity would come off once the targeted radiotherapeutic agent is in the body due to the action of endogenous deiodinases, and would not be localized with the HER2-binding agent at the targeted cells. Although this can, in some embodiments, be minimized by conducting the radioiodination at a lower pH (4-5), it cannot be completely avoided. One approach to avoid this potential problem is to introduce non-radioactive iodine onto those tyrosine residues first, before subjecting the macromolecule to radioiodination. It is highly likely that, mediated by these same endogenous deiodinases, the nonradioactive iodine on the constituent tyrosine residues would be removed, thereby restoring the original tyrosine structure and maintaining the affinity of the HER2-binding agent for the envisioned target. Non-radioactive iodination of the HER2-binding agent can be accomplished. In some embodiments, by treating the HER2-binding agent with an excess of sodium iodide in the presence of an oxidizing agent (e.g., Chloramine-T).

### V. Pharmaceutical Compositions and Formulations

The disclosure also provides pharmaceutical compositions comprising a HER2-binding agent or nucleic acid disclosed herein and a pharmaceutically acceptable carrier. In some embodiments, the disclosure provides pharmaceutical compositions that comprise a therapeutically effective amount of a HER2-binding agent or nucleic acid described herein and a pharmaceutically acceptable carrier. The pharmaceutical compositions comprise any suitable and pharmaceutically acceptable carrier, diluent, adjuvant or buffer solution.

The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science (1990) 249:1527-33).

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, e.g., sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In some embodiments, a pharmaceutical composition may contain nanoparticles, e.g., polymeric nanoparticles, liposomes, or micelles (See Anselmo et al., Bioeng Transl Med. (2016) 1:10-29).

In some embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate, or poly-D(−)-3-hydroxybutyric acid. Sustained release compositions may also include liposomes that can be prepared by any of several methods known in the art.

Pharmaceutical compositions containing a HER2-binding agent disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a recombinant human sialidase, a recombinant human sialidase fusion protein, or an antibody conjugate disclosed herein is administered by IV infusion. In certain embodiments, a recombinant human sialidase, a recombinant human sialidase fusion protein, or an antibody conjugate disclosed herein is administered by intratumoral injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

An intravenous drug delivery formulation may be contained in a syringe, pen, or bag. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation and stored as about 250 mg/vial to about 1,000 mg/vial. In certain embodiments, the formulation may be a liquid formulation and stored as about 600 mg/vial. In certain embodiments, the formulation may be a liquid formulation and stored as about 250 mg/vial.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, in certain embodiments between 5 and 9 or between 6 and 8, and in particular embodiments between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g., 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes 1-1.5 mg/ml of citric acid, 0.25 to 0.5 mg/ml of sodium citrate, 1.25 to 1.75 mg/ml of disodium phosphate dihydrate, 0.7 to 1.1 mg/ml of sodium dihydrogen phosphate dihydrate, and 6.0 to 6.4 mg/ml of sodium chloride. In certain embodiments, the pH of the formulation is adjusted with sodium hydroxide.

A polyol, which acts as a tonicifier and may stabilize the multi-specific binding protein, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to 15 mg/ml. In certain embodiments, the concentration of mannitol may be about 10-14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

The multi-specific binding protein may be lyophilized to produce a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5. In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5,000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta (2001) 308:43-53; Steimer et al., Clinica Chimica Acta (2001) 308:33-41).

In general, dosages based on body weight are from about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, or about 50 mg to about 100 mg/kg of body weight.

Doses may be administered one or more times daily, weekly, monthly or yearly. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The pharmaceutical composition may be administered as needed to subjects. In some embodiments, an effective dose of the pharmaceutical composition is administered to a subject one or more times. In various embodiments, an effective dose of the pharmaceutical composition is administered to the subject once a month, less than once a month, such as, for example, every two months, every three months, or every six months. In other embodiments, an effective dose of the pharmaceutical composition is administered more than once a month, such as, for example, every two weeks, every week, twice per week, three times per week, daily, or multiple times per day. An effective dose of the pharmaceutical composition is administered to the subject at least once. In some embodiments, the effective dose of the pharmaceutical composition is administered multiple times, including for periods of at least a month, at least six months, or at least a year. In some embodiments, the pharmaceutical composition is administered to a subject as needed to alleviate one or more symptoms of a condition.

In some embodiments, the disclosure provides a HER2-binding agent for use in medicine. In some embodiments, the disclosure provides a HER2-binding agent for use in treatment of a disease. In certain embodiments, the disclosure provides a HER2-binding agent for use in treatment of cancer.

In some embodiments, the disclosure provides use of a HER2-binding agent in the manufacture of a medicament for the treatment of a disease in a patient in need thereof. In some embodiments, the disclosure provides use of a HER2-binding agent in the manufacture of a medicament for the treatment of cancer in a patient in need thereof. In some embodiments, the disclosure provides use of a HER2-binding agent in the manufacture of a medicament for the treatment of HER2⁺ cancer in a patient in need thereof. In certain embodiments, the disclosure provides use of a HER2-binding agent in the manufacture of a medicament for the treatment of a disease in a patient in need thereof.

### VI. Methods of Use

In some embodiments, the disclosure provides the use of a HER2-binding agent (including a labeled HER2-binding agent) for treatment or diagnosis of a disease or condition in a subject, and use of a HER2-binding agent (including a labeled HER2-binding agent) in the preparation of a medicament for treatment or diagnosis of a disease or condition in a subject.

In some embodiments, the disclosure provides a method of detecting HER2⁺ cells in a subject, the method comprising: a) administering a labeled HER2-binding agent or pharmaceutical composition provided herein to the subject; and b) detecting binding of the labeled HER2-binding agent to HER2⁺ cells in the subject, wherein the detection of the binding indicates the presence of HER2⁺ cells. In some embodiments, the HER2⁺ cells are HER2⁺ tumor cells. In some embodiments, the subject has cancer. In some embodiments, the subject is a human or a non-human primate. In some embodiments, the non-human primate is a cynomolgus monkey or a rhesus monkey.

In some embodiments, the disclosure provides a method of imaging a site of disease in a subject, comprising administering a labeled HER2-binding agent or pharmaceutical composition provided herein to the subject. In some embodiments, imaging HER2⁺ cells in the subject comprises performing a positron emission tomography (PET) scan or positron emission tomography/computed tomography (PET/CT) scan on the subject. In some embodiments, the HER2⁺ cells are HER2⁺ tumor cells. In some embodiments, the subject has cancer. In some embodiments, the subject is a human or a non-human primate. In some embodiments, the non-human primate is a cynomolgus monkey or a rhesus monkey.

In some embodiments, the disclosure provides a method of diagnosing a disease or medical condition in a subject comprising administering a labeled HER2-binding agent or pharmaceutical composition provided herein to the subject. In some embodiments, the subject has cancer. In some embodiments, the subject is a human or a non-human primate. In some embodiments, the non-human primate is a cynomolgus monkey or a rhesus monkey.

In some embodiments, the disclosure provides a method of detecting HER2⁺ cells in a subject, the method comprising: a) administering a labeled HER2-binding agent, to the subject; and b) detecting binding of the labeled HER2-binding agent to HER2⁺ cells in the subject, wherein the detection of the binding indicates the presence of HER2⁺ cells. In some embodiments, detecting binding of the labeled HER2-binding agent to HER2⁺ cells in the subject comprises imaging HER2⁺ cells in the subject. In some embodiments, imaging HER2⁺ cells in the subject comprises performing a positron emission tomography (PET) scan or positron emission tomography/computed tomography (PET/CT) scan on the subject. In some embodiments, the HER2⁺ cells are HER2⁺ T cells. In some embodiments, the HER2⁺ cells are HER2⁺ tumor cells. In some embodiments, the detecting is carried out within about 1 day or less (e.g., within about 6 hours, 4 hours, 2 hours, 90 minutes, 1 hour, 30 minutes or less) after the administering. In some embodiments, the method is repeated for one or more times, such as about 1 to 4 times per year. In some embodiments, the method is repeated after about 1 day after the prior administration of the HER2-binding agent. In some embodiments, the method is repeated for more than 1 year. In some embodiments, the method has a sensitivity of about 1 nM to about 30 nM. In some embodiments, the subject is a human or a non-human primate. In some embodiments, the subject is a cynomolgus monkey or a rhesus monkey. In some embodiments, the subject is human. In some embodiments, the subject has cancer.

In some embodiments, the HER2-binding agents provided herein are used to investigate the nature of a disease condition in a patient. The HER2-binding agents may be used to prepare images of sites of disease in the body of a subject using imaging techniques such as X-ray, gamma-ray, or PET scanning, or similar. In some embodiments, the HER2-binding agents provided herein are used in a method of imaging a site of disease in a subject, which comprises administering a suitably detectably labelled HER2-binding agent provided herein to a subject and scanning the subject's body subsequently. Alternatively, administering a HER2-binding agent provided herein to a subject can provide for a test result by analyzing a sample from the subject following administration of the molecule. Such embodiments may include a method of diagnosis of a disease or medical condition in a subject comprising administration of a HER2-binding agent provided herein. The embodiment includes methods of detecting a site of disease or medical condition and the use of HER2-binding agents provided herein in such methods.

In some embodiments, the HER2-binding agents provided herein are suitable for treating a disease or disorder. In some embodiments, the disease or disorder is a hyperproliferative disease or disorder. In some embodiments, the disease or disorder is cancer. In certain embodiments the disease or disorder is a HER2⁺ cancer. In some embodiments, the cancer is selected from breast cancer, ovarian cancer, cervical cancer, endometrial cancer, head and neck cancer, brain cancer, gastric cancer (e.g., colorectal and colon cancer), pancreatic cancer, renal cancer, prostate cancer, lung cancer (e.g., NSCLC), bladder cancer, urothelial cancer, an epidermal-derived cancer, and/or a metastasis originating therefrom. In some embodiments, the cancer is selected from HER2 expressing breast cancer, ovarian cancer, gastrointestinal cancer, bladder cancer, and pancreatic cancer. In particular embodiments, the HER2-binding agents provided herein are suitable for treating a metastasis. In particular embodiments, the HER2-binding agents provided herein are suitable for the treatment of breast cancer. In certain embodiments, the HER2-binding agents provided herein are suitable for treating a breast cancer metastasis. In particular embodiments, the HER2-binding agent is suitable for treating breast cancer that has metastasized to the brain.

In some embodiments, a HER2-binding agent provided herein is suitable for the treatment of a HER2⁺ cancer in a patient for whom a treatment with trastuzumab or pertuzumab is not eligible. Typically, the patient is scored HER2 (1+), HER2 (2+) or HER2 (3+) by immunohistochemistry. Accordingly, in some embodiments, the disclosure provides a method for treating a HER2⁺ cancer in a patient in need thereof comprising the steps consisting of i) staging the patient for its HER2 expression and ii) administering the patient with an effective amount of a HER2-binding agent provided herein when the patient is considered at step i) as HER2⁺ or HER2⁺⁺. Typically, testing is usually done at the same time as the initial biopsy or cancer surgery. Samples of cancer tissue from previous biopsies or an earlier surgery also may also be used. One skilled in the art can easily select the most appropriate method for staging the patient for its HER2 expression. Typically, the two main methods used for HER2 testing are immunohistochemistry (IHC) and fluorescence in-situ hybridization (FISH). 0-1+ means that a normal amount of the HER2 protein is present and the result is HER2-negative. 2+ means that a moderate amount of the HER2 protein is present. 3+ means that there is a higher-than-normal level of HER2 protein and the result is HER2-positive. By way of example, the method of the present disclosure typically comprises the step of i) determining the level of HER2 in tumor sample obtained from the patient, ii) comparing the level determined at step i) with a predetermined reference level and iii) administering the patient with a therapeutically effective amount of a HER2-binding agent provided herein when the level determined at step i) is lower than the predetermined reference value.

In some embodiments, the disclosure provides a HER2-binding agent, or a pharmaceutical composition provided herein for use in medicine. In some embodiments, the disclosure provides the use of a HER2-binding agent or a pharmaceutical composition provided herein in the manufacture of a medicament for the treatment of a disease in a patient in need thereof.

In some embodiments, the disclosure provides a method of imaging a site of disease in a subject, that comprises administering a detectably labelled HER2-binding agent or a pharmaceutical composition provided herein to a subject.

In some embodiments, the disclosure provides a method of diagnosis of a disease or medical condition in a subject comprising administering a HER2-binding agent or a pharmaceutical composition provided herein to a subject.

In some embodiments, the disclosure provides a method of treating a disease in a subject in need thereof comprising administering to the subject a therapeutically effective amount of a HER2-binding agent or a pharmaceutical composition provided herein.

In some embodiments, the disclosure provides a method of treating a HER2⁺ cancer in a subject in need thereof comprising administering to the subject a therapeutically effective amount of a HER2-binding agent or pharmaceutical composition provided herein. In some embodiments, the subject is a HER2⁺ or HER2⁺⁺ subject.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive (e.g., synergistic). The delivery can be such that an effect of the first treatment delivered is still detectable when the second is delivered.

In some embodiments, the disclosure provides a method of treating a subject by the administration of a second therapeutic agent in combination with a HER2-binding agent described herein.

Exemplary therapeutic agents that can be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

### VII. Kits and Articles of Manufacture

Further provided herein are kits and articles of manufacture comprising a HER2-binding agent provided herein, such as a labeled HER2-binding agent. In some embodiments, the kit or article of manufacture comprises an instruction for use of the HER2-binding agent according to any of the methods described above.

Also provided herein are methods for the prevention and/or treatment of a disease or condition in a subject, such as a cancer, that includes administering to a subject engineered cells comprising a CAR provided herein.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

The description above describes multiple embodiments of the invention. The patent application specifically contemplates all combinations and permutations of the embodiments. According to the present disclosure, back-references in the dependent claims are meant as short-hand writing for a direct and unambiguous disclosure of each and every combination of claims that is indicated by the back-reference. Any compound disclosed herein can be used in any of the treatment methods herein, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Generation of HER2-Binding VHH Domains

The development of VHH polypeptides that specifically bind HER2 was achieved by multiple methods. Internal ligand discovery was accomplished through phage screening of proprietary naïve llama and alpaca VHH libraries against recombinant HER2-Fc and HER2-DDDDK proteins. Libraries were initially pre-cleared using a counter screen against Fc and DDDDK protein sequences displayed on magnetic beads to ensure target specificity.

A total of 16 different screening conditions were utilized. The proprietary llama and alpaca VHH phage libraries were screened against HER2 target proteins. The HER2 target proteins used were recombinant forms of human HER2 with a C-terminal tag composed of either a human IgG1 Fc domain or DDDDK (SEQ ID NO: 207) amino acid sequence. Target proteins were immobilized onto magnetic beads through affinity linkage using the Fc or DDDDK (SEQ ID NO: 207) tags. Phage selections were completed in two different blocking solutions: 3% BSA or 3% milk in PBS. Binding interactions between the target protein and phage libraries or selection pools were assessed for various times (15, 60, or 120 minutes). Three rounds of phage selection were done with each selection strategy and clones were usually isolated from the round three phage pools.

The final isolated phage selection pools were evaluated in a 96-well ELISA assay against HER2-Fc as well as a control Fc protein. A total of 355 positive phagemid clones were identified as positive binders, defined as exhibiting >150% higher binding over background, and submitted for sequencing. Sequence analysis resulted in 87 unique VHH sequences being identified with HER2-binding affinity. Plasmids for these positive phagemid clones were then transferred into SS320 E. coli for direct protein expression and purification to directly evaluate protein affinity. Purified VHH were then retested in a human HER2-Fc and human HER2-His6 indirect ELISA format, resulting in 24 VHH with affinity stronger than 150 nM. Specificity was verified by assaying binding affinity to other human ErBB receptors, including EGFR, HER3, and HER4. Cross-species reactivity was characterized in murine, rat, canine, rhesus, and cynomolgus analogues of HER2-Fc. Top hits were further modified and evolved to produce higher affinity binding proteins and improve their compatibility and performance as radiolabeled compounds for targeted radiotherapy.

VHH sequences were also evaluated to identify the corresponding framework regions (FR) and complementary determining regions (CDR). In general, the linear sequence of a VHH protein corresponds to the following structural sequence: FR1-CDR1-FR2-CDR2-FR3-CDR3-FR4. To ensure maximal performance of VHH proteins as targeted drug conjugates (e.g., radioligands or other types of drug conjugates), all amino acids in the CDR regions that could provide sites of radiolabeling conjugations (or other types of drug conjugations) were mutated to prevent such conjugations. In addition, amino acids in the framework regions within five amino acids of CDRs were also modified to prevent steric interference with the binding pocket of the VHH post radiolabeling (or any other type of drug conjugation). Such sequence modifications ensured ligands maintained optimal binding and internalization characteristics required as radioligands. Sequence modifications included: the replacement of: lysine (K) residues with either arginine (R) or histidine (H) residues; the replacement of unpaired cysteine (C) residues with alanine (A) or serine (S) residues; or the replacement of glutamine (Q) residues with asparagine (N).

Identification of the structural CDR sequences for VHH_106 to VHH_172 is provided in Table 2, as well as exemplary C-terminal peptide fusions.

TABLE 2
Exemplary HER2 binding sequences

| VHH | | |
|---|---|---|
| ID | CDR Sequences | VHH Amino Acid Sequence |
| 106 | CDR1: GFEFSNYD (SEQ ID NO: 82) | QVHLVESGGGLVQPGGSLRLSCVASGFEFSNYDM |
| | CDR2: STRGRTT (SEQ ID NO: 102) | SWVRQAPGKGLEWVSTISTRGRTTYGYSAKGRFT |
| | CDR3: NKAIEKSTRPP (SEQ ID NO: | ISRDNAKNTLYLQMDSLESEDTAKYYCNKAIEKS |
| | 124) | TRPPGQGTQVTVSS (SEQ ID NO: 1) |
| 107 | CDR1: GSIFSINV (SEQ ID NO: 83) | QVQLVESGGGLVQPGGSLRLSCAASGSIFSINVM |
| | CDR2: TSGGSTN (SEQ ID NO: 103) | GWYRQAPGMQRQLVAIITSGGSTNYADSVKGRFT |
| | CDR3: ATIHVRESTWYYAY (SEQ ID NO: | VSRDNAKNTLYLRMNSLKPEDTAVYYCATIHVRE |
| | 125) | STWYYAYWGQGTQVTVSS (SEQ ID NO: 2) |
| 108 | CDR1: GRTESSYA (SEQ ID NO: 84) | QVQLVESGGGLVQAGGSLRLSCAASGRTESSYAM |
| | CDR2: ARGGVSTG (SEQ ID NO: 104) | GWFRQAPGLEREFVAAIARGGVSTGYADSVKGRF |
| | CDR3: ATYFWGSPHY (SEQ ID NO: | AISRDNAKNMVYLQMNSLKPEDTAVYYCATYFWG |
| | 126) | SPHYWGQGTQVTVSS (SEQ ID NO: 3) |
| 109 | CDR1: GFTEDDYG (SEQ ID NO: 85) | QVQLVESGGGLVQPGGSLRLSCEASGFTEDDYGM |
| | CDR2: TWNGGNPN (SEQ ID NO: 105) | SWVRQAPGKGLEWVSGITWNGGNPNYADSVKGRF |
| | CDR3: QRGYSRGYMY (SEQ ID NO: | TISRDNAKRTLYLQMNSLKAEDTAVYYCQRGYSR |
| | 127) | GYMYWGQGTQVTVSS (SEQ ID NO: 4) |
| 110 | CDR1: GLGFSALA (SEQ ID NO: 86) | AQVQLVESGGGSVQLGGSLRLSCAVSGLGESALA |
| | CDR2: SRTGGTTY (SEQ ID NO: 106) | MGWFRQAPGKERERVAAISRTGGTTYYTDSVKGR |
| | CDR3: ASRYTALLSTTARGYDY (SEQ ID | FTISRDNGKNMVYLQMNSLKPEDAAVYYCASRYT |
| | NO: 128) | ALLSTTARGYDYWGQGTQVTVSS |
| | | (SEQ ID NO: 5) |
| 111 | CDR1: GIIFRITT (SEQ ID NO: 87) | AQVQLVESGGGLVQSGGSLRLSCAASGIIFRLTT |
| | CDR2: TSGASTN (SEQ ID NO: 117) | MDWYRQAPGKQRELVATITSGASTNYADSVKGRF |
| | CDR3: YARRSYWASDY (SEQ ID NO: | TISRDNAKNTMYLQMNSLKAEDTAVYICYARRSY |
| | 129) | WASDYWGKGTLVTVSS (SEQ ID NO: 6) |
| 112 | CDR1: GLDESALA (SEQ ID NO: 88) | AQVQLVESGGGSVQLGGSLRLSCAVSGLDESALA |
| | CDR2: SRTGGTT (SEQ ID NO: 118) | MGWFRQAPGKERERVAAISRTGGTTYCTDSVKGR |
| | CDR3: ASRYTALLSTTAREYDY (SEQ ID | FTISRDNGKNMVYLQMNSLKPEDAAVYYCASRYT |
| | NO: 130) | ALLSTTAREYDYWGQGTLVTVSS |
| | | (SEQ ID NO: 7) |
| 113 | CDR1: EFTSDNFA (SEQ ID NO: 89) | AQVQLVESGGGLVQPGGSLRLSCAASEFTSDNFA |
| | CDR2: SSDGRMKN (SEQ ID NO: 108) | IGWFRQAPGKEREGVSCISSDGRMKNYADSVKGR |
| | CDR3: AAARVGAEYVCYEAYSAWYD(SEQ | FTISRDNADKAVYLQMNSLKPEDTATYYCAAARV |
| | ID NO: 131) | GAEYVCYEAYSAWYDYWGQGTQVTVSS(SEQ |
| | | ID NO: 8) |
| 114 | CDR1: GRTESSY (SEQ ID NO: 90) | AEVQLVESGGGLVQAGGSPRLSCAASGRTFSSYH |
| | CDR2: TWSGGSTY (SEQ ID NO: 109) | MGWFRQAPRKEREFVAAITWSGGSTYYADFVKGR |
| | CDR3: AVGKDFYGIIMRGEYDY (SEQ ID | FTISRDNAKNTVYLQMNSLKPEDTAVYYCAVGKD |
| | NO: 132) | FYGIIMRGEYDYWGQGTQVTVSS |
| | | (SEQ ID NO: 9) |
| 115 | CDR1: GFPFSSYD (SEQ ID NO: 91) | AQVQLVESGGGLVQPGGSLRVSCAASGEPFSSYD |
| | CDR2: SPDGRSTF (SEQ ID NO: 119) | MRWVRQAPGKGLEWVSAISPDGRSTFYADSVKGR |
| | CDR3: ARGVLDY (SEQ ID NO: 137) | FTISRDNLKNTLYLQMNSLKPEDTALYYCARGVL |
| | | DYWGQGTKVTVSS (SEQ ID NO: 10) |
| 116 | CDR1: GRIFSGWV (SEQ ID NO: 92) | AEVQLVESGGGSVQAGGSLRLSCAASGRIFSGWV |
| | CDR2: RWSSGVAT (SEQ ID NO: 120) | MGWFREAPGKEREFVAFIRWSSGVATYADSVKGR |
| | CDR3: NLGIIYSSGTYQPGSS (SEQ ID | FDLSIDYAKNTVYLQMNALKPEDTAVYYCNLGII |
| | NO: 138) | YSSGTYQPGSSWGQGTLVTVSS |
| | | (SEQ ID NO: 11) |
| 117 | CDR1: GLSLRPLA (SEQ ID NO: 93) | AQVQLVESGGGLAQPGGSLRLSCAVSGLSLRPLA |
| | CDR2: TFSGSTK (SEQ ID NO: 114) | IGWFRQAPGKPRELVAIVTFSGSTKYASSVKGRF |
| | CDR3: SAGRTYGGNTYVADY (SEQ ID | TISRDTAENTVYLQMNSLKPEDTAVYQCSAGRTY |
| | NO: 206) | GGNTYVADYWGQGTLVTVSS |
| | | (SEQ ID NO: 12) |
| 118 | CDR1: EFTESNYA (SEQ ID NO: 94) | AEVQLVESGGGLVQPGGSLRLSCAASEFTESNYA |
| | CDR2: NWSGGITT (SEQ ID NO: 115) | MSWVRQAPGKGLEWVSAINWSGGITTYADSVKGR |
| | CDR3: AKGWGTTTRDTGL (SEQ ID NO: | FTISRDNAKNTLHLQMNSLKPEDTAVYYCAKGWG |
| | 140) | TTTRDTGLLSQGTLVTVSS |
| | | (SEQ ID NO: 13) |
| 119 | CDR1: GSVFSILS (SEQ ID NO: 95) | AQVQLVESGGGLVQLGGSLRLSCEVSGSVESILS |
| | CDR2: SGRETA (SEQ ID NO: 121) | MGWYRQAPGKQRELVALLSGRETASHESVKGRET |
| | CDR3: NLNAREIDVGQYRDY (SEQ ID | LSRDDASDKVILQLNSLKPEDTAVYYCNLNAREI |
| | NO: 141) | DVGQYRDYWGQGTQVTVSS (SEQ ID NO: |
| | | 14) |
| 120 | CDR1: GRTESTYN (SEQ ID NO: 96) | AQVQLVESGGGLVQAGGSLRLSCTASGRTFSTYN |
| | CDR2: MWSGGSTH (SEQ ID NO: 122) | VGWFRQAPGKEREFVAAIMWSGGSTHYADSVKGR |
| | CDR3: VNLPWSRNLEG (SEQ ID NO: | FTISRDNAKNMLYLQMNSLKPEDTALYYCVNLPW |
| | 142) | SRNLEGWGPGTQVTVSS (SEQ ID NO: 15) |
| 121 | CDR1: GRADVINV (SEQ ID NO: 97) | AEVQLVESGGGSGQPGGSLSVSCVVSGRADVINV |
| | CDR2: TVGGTY (SEQ ID NO: 123) | MGWYRQAPGKERELVAAITVGGTYYADAVRGRFT |
| | CDR3: NMNEYRVATPTTGQQKFEY(SEQ | ISRDNAKNTLYLQMNSLKPEDTAVYICNMNEYRV |
| | ID NO: 143) | ATPTTGQQKFEYWGQGTQVTVSS |
| | | (SEQ ID NO: 16) |
| 122 | CDR1: GLGFSALA (SEQ ID NO: 86) | AQVQLVESGGGSVQLGGSLRLSCAVSGLGFSALA |
| | CDR2: SRTGGTTY (SEQ ID NO: 106) | MGWFRQAPGKERERVAAISRTGGTTYYTDSVKGR |
| | CDR3: ASRYTALLSTTAREYDY (SEQ ID | FTISRDNGKNMVYLQMNSLKPEDAAVYYCASRYT |
| | NO: 130) | ALLSTTAREYDYWGQGTQVTVSS |
| | | (SEQ ID NO: 17) |
| 123 | CDR1: GRTENSYFYT (SEQ ID NO: 98) | AQVQLVESGGGLVQAGGSLRLSCAASGRTENSYE |
| | CDR2: ARMNWNADST (SEQ ID NO: | YTMAWFRQAPGKEREFVARMNWNADSTYHADSVK |
| | 107) | GRFTISRENTVYLQMNSLEPEDTAVYYCAAKSIS |
| | CDR3: AAKSISAPKYDY (SEQ ID NO: | APKYDYWGQGTQVTVSS (SEQ ID NO: 18) |
| | 144) | |
| 124 | CDR1: EFTSDNFA (SEQ ID NO: 89) | AQVQLVESGGGLVQPGGSLRLSCAASEFTSDNFA |
| | CDR2: SSDGRMKN (SEQ ID NO: 108) | IGWFRQAPGKEREGVSCISSDGRMKNYADSVKGR |
| | CDR3: AAARVGAEYVCYEAYSAWYD(SEQ | LTISRDNADKTVYLQMNSLKPEDTATYYCAAARV |
| | ID NO: 131) | GAEYVCYEAYSAWYDHWGQGTQVTVSS(SEQ |
| | | ID NO: 19) |
| 125 | CDR1: GRTESSYH (SEQ ID NO: 99) | AQVQLVESGGGLVQAGGSPRLSCAASGRTFSSYH |
| | CDR2: TWSGGSTY (SEQ ID NO: 109) | MGWFRQAPRKEREFVAAITWSGGSTYYADFVKGR |
| | CDR3: AVGKDFYGIIMRGEYDY (SEQ ID | FTISRDNAKNTVYLQMNSLKPEDTAVYYCAVGKD |
| | NO: 132) | FYGIIMRGEYDYWGQGTLVTVSS |
| | | (SEQ ID NO: 20) |
| 126 | CDR1: GSIFGENV (SEQ ID NO: 100) | AEVQLVESGGGLVQAGGSLRLSCAASGSIFGEN |
| | CDR2: GIGTSAT (SEQ ID NO: 110) | VMEWYRQAPGKQRELVAVLGIGTSATYADSVKG |
| | CDR3: NAKFVY (SEQ ID NO: 133) | RFTISRDNAKNTVYLQMNSLKPEDTAVYYCNAK |
| | | FVYWGQGTLVTVSS (SEQ ID NO: 21) |
| 127 | CDR1: GLGFSALA (SEQ ID NO: 86) | AQVQLVESGGGSVQLGGSLRLSCAVSGLGFSALA |
| | CDR2: SRTGGTTY (SEQ ID NO: 106) | MGWFRQAPGKERERVAAISRTGGTTYYTDSVKGR |
| | CDR3: ASRYTALLSTTARKYDY (SEQ ID | FTISRDNGKNMVYLQMNSLKPEDAAVYYCASRYT |
| | NO: 134) | ALLSTTARKYDYWGQGTQVTVSS |
| | | (SEQ ID NO: 22) |
| 128 | CDR1: GITFSINT (SEQ ID NO: 101) | EVQLVESGGGLVQAGGSLRLSCAASGITESINTM |
| | CDR2: LISSIGDTYYADSVRG (SEQ ID | GWYRQAPGKQRELVALISSIGDTYYADSVRGRET |
| | NO: 111) | ISRDNAKNTVYLQMNSLKPEDTAVYYCKRERTAA |
| | CDR3: KRFRTAAQGTDY (SEQ ID NO: | QGTDYWGQGTQVTVSS (SEQ ID NO: 23) |
| | 135) | |
| 129 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVKGRET |
| | NO: 111) | ISRDNAKNTVYLQMNSLKPEDTAVYYCRRERTAA |
| | CDR3: RRFRTAAQGTDY (SEQ ID NO: | QGTDYWGQGTQVTVSS (SEQ ID NO: 24) |
| | 136) | |
| 130 | CDR1: GITESINT (SEQ ID NO: 101) | EVQLVESGGGLVQAGGSLRLSCAASGITESINTM |
| | CDR2: LISSIGDTYYADSVRG (SEQ ID | GWYRQAPGKQRELVALISSIGDTYYADSVRGRFT |
| | NO: 111) | ISRDNAKNTVYLQMNSLKPEDTAVYYCRRERTAA |
| | CDR3: RRERTAAQGTDY (SEQ ID NO: | QGTDYWGQGTQVTVSS (SEQ ID NO: 25) |
| | 136) | |
| 131 | CDR1: GITFSINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVRGRET |
| | NO: 111) | ISRDNAKNTVYLQMNSLRPEDTAVYYCRRERTAA |
| | CDR3: RRFRTAAQGTDY (SEQ ID NO: | QGTDYWGQGTQVTVSS (SEQ ID NO: 26) |
| | 136) | |
| 132 | CDR1: GITESINT (SEQ ID NO: 101) | EVQLVESGGGLVQAGGSLRLSCAASGITESINTM |
| | CDR2: LISSIGDTYYADSVRG (SEQ ID | |
| | NO: 111) | GWYRQAPGKQRELVALISSIGDTYYADSVRGRET |
| | | ISRDNARNTVYLQMNSLKPEDTAVYYCRRERTAA |
| | CDR3: RRFRTAAQGTDY (SEQ ID NO: | QGTDYWGQGTQVTVSS (SEQ ID NO: 27) |
| | 136) | |
| 133 | CDR1: GITESINT (SEQ ID NO: 101) | EVQLVESGGGLVQAGGSLRLSCAASGITESINTM |
| | CDR2: LISSIGDTYYADSVRG (SEQ ID | GWYRQAPGRQRELVALISSIGDTYYADSVRGRET |
| | NO: 111) | ISRDNAKNTVYLQMNSLKPEDTAVYYCRRERTAA |
| | CDR3: RRERTAAQGTDY (SEQ ID NO: | QGTDYWGQGTQVTVSS (SEQ ID NO: 28) |
| | 136) | |
| 134 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVRGRFT |
| | NO: 111) | ISRDNARNTVYLQMNSLRPEDTAVYYCRRERTAA |
| | CDR3: RRFRTAAQGTDY (SEQ ID NO: | QGTDYWGQGTQVTVSS (SEQ ID NO: 29) |
| | 136) | |
| 135 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGRQRELVALISSIGDTYYADSVRGRFT |
| | NO: 111) | ISRDNAKNTVYLQMNSLRPEDTAVYYCRRERTAA |
| | CDR3: RRERTAAQGTDY (SEQ ID NO: | QGTDYWGQGTQVTVSS (SEQ ID NO: 30) |
| | 136) | |
| 136 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGRQRELVALISSIGDTYYADSVRGRET |
| | NO: 111) | ISRDNARNTVYLQMNSLKPEDTAVYYCRRFRTAA |
| | CDR3: RRERTAAQGTDY (SEQ ID NO: | QGTDYWGQGTQVTVSS (SEQ ID NO: 31) |
| | 136) | |
| 137 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGRQRELVALISSIGDTYYADSVRGRET |
| | NO: 111) | ISRDNARNTVYLQMNSLRPEDTAVYYCKRERTAA |
| | CDR3: KRFRTAAQGTDY (SEQ ID NO: | QGTDYWGQGTQVTVSS (SEQ ID NO: 32) |
| | 135) | |
| 138 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGRQRELVALISSIGDTYYADSVKGRFT |
| | NO: 111) | ISRDNARNTVYLQMNSLRPEDTAVYYCRRERTAA |
| | CDR3: RRERTAAQGTDY (SEQ ID NO: | QGTDYWGQGTQVTVSS (SEQ ID NO: 33) |
| | 136) | |
| 139 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVRGRET |
| | NO: 111) | ISRDNAKNTVYLQMNSLKPEDTAVYYCKRERTAA |
| | CDR3: KRFRTAAQGTDY (SEQ ID NO: | QGTDYWGQGTQVTVSSGGCG |
| | 135) | (SEQ ID NO: 34) |
| 140 | CDR1: GITFSINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVRGRET |
| | NO: 111) | ISRDNAKNTVYLQMNSLKPEDTAVYYCKRFRTAA |
| | CDR3: KRFRTAAQGTDY (SEQ ID NO: | QGTDYWGQGTQVTVSSGGKG |
| | 135) | (SEQ ID NO: 35) |
| 141 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVRGRET |
| | NO: 111) | ISRDNAKNTVYLQMNSLKPEDTAVYYCKRERTAA |
| | CDR3: KRFRTAAQGTDY (SEQ ID NO: | QGTDYWGQGTQVTVSSGGC |
| | 135) | (SEQ ID NO: 36) |
| 142 | CDR1: GITFSINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVRGRET |
| | NO: 111) | |
| | CDR3: KRFRTAAQGTDY (SEQ ID NO: | ISRDNAKNTVYLQMNSLKPEDTAVYYCKRERTAA |
| | 135) | QGTDYWGQGTQVTVSSGGFKGGKG |
| | | (SEQ ID NO: 37) |
| 143 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVRGRET |
| | NO: 111) | ISRDNAKNTVYLQMNSLKPEDTAVYYCKRERTAA |
| | CDR3: KRFRTAAQGTDY (SEQ ID NO: | QGTDYWGQGTQVTVSSGGFKGGC |
| | 135) | (SEQ ID NO: 38) |
| 144 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVRGRFT |
| | NO: 111) | ISRDNAKNTVYLQMNSLKPEDTAVYYCRRERTAA |
| | CDR3: RRFRTAAQGTDY (SEQ ID NO: | QGTDYWGQGTQVTVSSGGC |
| | 136) | (SEQ ID NO: 39) |
| 145 | CDR1: GITESINT (SEQ ID NO: 101) | EVQLVESGGGLVQAGGSLRLSCAASGITFSINTM |
| | CDR2: LISSIGDTYYADSVRG (SEQ ID | GWYRQAPGKQRELVALISSIGDTYYADSVRGRFT |
| | NO: 111) | ISRDNAKNTVYLQMNSLKPEDTAVYYCRRFRTAA |
| | CDR3: RRFRTAAQGTDY (SEQ ID NO: | QGTDYWGQGTQVTVSSGGKG |
| | 136) | (SEQ ID NO: 40) |
| 146 | CDR1: GITESINT (SEQ ID NO: 101) | EVQLVESGGGLVQAGGSLRLSCAASGITESINTM |
| | CDR2: LISSIGDTYYADSVRG (SEQ ID | GWYRQAPGKQRELVALISSIGDTYYADSVRGRFT |
| | NO: 111) | ISRDNAKNTVYLQMNSLKPEDTAVYYCRRERTAA |
| | CDR3: RRFRTAAQGTDY (SEQ ID NO: | QGTDYWGQGTQVTVSSGGFKGGKG |
| | 136) | (SEQ ID NO: 41) |
| 147 | CDR1: GITESINT (SEQ ID NO: 101) | EVQLVESGGGLVQAGGSLRLSCAASGITESINTM |
| | CDR2: LISSIGDTYYADSVRG (SEQ ID | GWYRQAPGKQRELVALISSIGDTYYADSVRGRFT |
| | NO: 111) | ISRDNAKNTVYLQMNSLKPEDTAVYYCRRERTAA |
| | CDR3: RRERTAAQGTDY (SEQ ID NO: | QGTDYWGQGTQVTVSSGGFKGGC |
| | 136) | (SEQ ID NO: 42) |
| 148 | CDR1: GFEFSNYD (SEQ ID NO: 82) | QVHLVESGGGLVQPGGSLRLSCVASGFEFSNYDM |
| | CDR2: STRGRTT (SEQ ID NO: 102) | SWVRQAPGKGLEWVSTISTRGRTTYGYSARGRFT |
| | CDR3: NKAIEKSTRPP (SEQ ID NO: | ISRDNAKNTLYLQMDSLESEDTAKYYCNKAIEKS |
| | 124) | TRPPGQGTQVTVSS (SEQ ID NO: 43) |
| 149 | CDR1: GFEFSNYD (SEQ ID NO: 82) | QVHLVESGGGLVQPGGSLRLSCVASGFEFSNYDM |
| | CDR2: STRGRTT (SEQ ID NO: 102) | SWVRQAPGKGLEWVSTISTRGRTTYGYSAKGRFT |
| | CDR3: NRAIEKSTRPP (SEQ ID NO: | ISRDNAKNTLYLQMDSLESEDTAKYYCNRAIEKS |
| | 145) | TRPPGQGTQVTVSS (SEQ ID NO: 44) |
| 150 | CDR1: GFEFSNYD (SEQ ID NO: 82) | QVHLVESGGGLVQPGGSLRLSCVASGFEFSNYDM |
| | CDR2: STRGRTT (SEQ ID NO: 102) | SWVRQAPGKGLEWVSTISTRGRTTYGYSARGRFT |
| | CDR3: NRAIEKSTRPP (SEQ ID NO: | ISRDNAKNTLYLQMDSLESEDTAKYYCNRAIEKS |
| | 145) | TRPPGQGTQVTVSS (SEQ ID NO: 45) |
| 151 | CDR1: GFEFSNYD (SEQ ID NO: 82) | QVHLVESGGGLVQPGGSLRLSCVASGFEFSNYDM |
| | CDR2: STRGRTT (SEQ ID NO: 102) | SWVRQAPGKGLEWVSTISTRGRTTYGYSAKGRFT |
| | CDR3: NKAIERSTRPP (SEQ ID NO: | ISRDNAKNTLYLQMDSLESEDTAKYYCNKAIERS |
| | 146) | TRPPGQGTQVTVSS (SEQ ID NO: 46) |
| 152 | CDR1: GFEFSNYD (SEQ ID NO: 82) | QVHLVESGGGLVQPGGSLRLSCVASGFEFSNYDM |
| | CDR2: STRGRTT (SEQ ID NO: 102) | SWVRQAPGKGLEWVSTISTRGRTTYGYSARGRFT |
| | CDR3: NKAIERSTRPP (SEQ ID NO: | ISRDNAKNTLYLQMDSLESEDTAKYYCNKAIERS |
| | 146) | TRPPGQGTQVTVSS (SEQ ID NO: 47) |
| 153 | CDR1: GFEFSNYD (SEQ ID NO: 82) | QVHLVESGGGLVQPGGSLRLSCVASGFEFSNYDM |
| | CDR2: STRGRTT (SEQ ID NO: 102) | SWVRQAPGKGLEWVSTISTRGRTTYGYSAKGRFT |
| | CDR3: NRAIERSTRPP (SEQ ID NO: | ISRDNAKNTLYLQMDSLESEDTAKYYCNRAIERS |
| | 147) | TRPPGQGTQVTVSS (SEQ ID NO: 48) |
| 154 | CDR1: GFEFSNYD (SEQ ID NO: 82) | QVHLVESGGGLVQPGGSLRLSCVASGFEFSNYDM |
| | CDR2: STRGRTT (SEQ ID NO: 102) | SWVRQAPGKGLEWVSTISTRGRTTYGYSARGRFT |
| | CDR3: NRAIERSTRPP (SEQ ID NO: | ISRDNAKNTLYLQMDSLESEDTAKYYCNRAIERS |
| | 147) | TRPPGQGTQVTVSS (SEQ ID NO: 49) |
| 155 | CDR1: EFTSDNFA (SEQ ID NO: 89) | AQVQLVESGGGLVQPGGSLRLSCAASEFTSDNFA |
| | CDR2: SSDGRMRN (SEQ ID NO: 112) | IGWFRQAPGKEREGVSCISSDGRMRNYADSVKGR |
| | CDR3: AAARVGAEYVCYEAYSAWYDY | FTISRDNADKAVYLQMNSLKPEDTATYYCAAARV |
| | (SEQ ID NO: 156) | GAEYVCYEAYSAWYDYWGQGTQVTVSS(SEQ |
| | | ID NO: 50) |
| 156 | CDR1: EFTSDNFA (SEQ ID NO: 89) | AQVQLVESGGGLVQPGGSLRLSCAASEFTSDNFA |
| | CDR2: SSDGRMKN (SEQ ID NO: 108) | IGWFRQAPGKEREGVSCISSDGRMKNYADSVRGR |
| | CDR3: AAARVGAEYVCYEAYSAWYDY(SEQ | FTISRDNADKAVYLQMNSLKPEDTATYYCAAARV |
| | ID NO: 156) | GAEYVCYEAYSAWYDYWGQGTQVTVSS(SEQ |
| | | ID NO: 51) |
| 157 | CDR1: EFTSDNFA (SEQ ID NO: 89) | AQVQLVESGGGLVQPGGSLRLSCAASEFTSDNFA |
| | CDR2: SSDGRMRN (SEQ ID NO: 112) | IGWFRQAPGKEREGVSCISSDGRMRNYADSVRGR |
| | CDR3: AAARVGAEYVCYEAYSAWYDY(SEQ | FTISRDNADKAVYLQMNSLKPEDTATYYCAAARV |
| | ID NO: 156) | GAEYVCYEAYSAWYDYWGQGTQVTVSS(SEQ |
| | | ID NO: 52) |
| 158 | CDR1: GRTFSSYH (SEQ ID NO: 99) | AEVQLVESGGGLVQAGGSPRLSCAASGRTFSSYH |
| | CDR2: TWSGGSTY (SEQ ID NO: 109) | MGWFRQAPRKEREFVAAITWSGGSTYYADFVRGR |
| | CDR3: AVGKDFYGIIMRGEYDY (SEQ ID | FTISRDNAKNTVYLQMNSLKPEDTAVYYCAVGKD |
| | NO: 132) | FYGIIMRGEYDYWGQGTQVTVSS |
| | | (SEQ ID NO: 53) |
| 159 | CDR1: GRTESSYH (SEQ ID NO: 99) | AEVQLVESGGGLVQAGGSPRLSCAASGRTESSYH |
| | CDR2: TWSGGSTY (SEQ ID NO: 109) | MGWFRQAPRKEREFVAAITWSGGSTYYADFVKGR |
| | CDR3: AVGRDFYGIIMRGEYDY (SEQ ID | FTISRDNAKNTVYLQMNSLKPEDTAVYYCAVGRD |
| | NO: 148) | FYGIIMRGEYDYWGQGTQVTVSS |
| | | (SEQ ID NO: 54) |
| 160 | CDR1: GRTESSY (SEQ ID NO: 90) | AEVQLVESGGGLVQAGGSPRLSCAASGRTESSYH |
| | CDR2: TWSGGSTY (SEQ ID NO: 109) | MGWFRQAPRKEREFVAAITWSGGSTYYADFVRGR |
| | CDR3: AVGRDFYGIIMRGEYDY (SEQ ID | FTISRDNAKNTVYLQMNSLKPEDTAVYYCAVGRD |
| | NO: 148) | FYGIIMRGEYDYWGQGTQVTVSS |
| | | (SEQ ID NO: 55) |
| 161 | CDR1: GLSLRPLA (SEQ ID NO: 93) | AQVQLVESGGGLAQPGGSLRLSCAVSGLSLRPLA |
| | CDR2: TFSGSTR (SEQ ID NO: 113) | IGWFRQAPGKPRELVAIVTFSGSTRYASSVKGRF |
| | CDR3: SAGRTYGGNTYVA (SEQ ID NO: | TISRDTAENTVYLQMNSLKPEDTAVYQCSAGRTY |
| | 139) | GGNTYVADYWGQGTLVTVSS (SEQ ID NO: |
| | | 56) |
| 162 | CDR1: GLSLRPLA (SEQ ID NO: 93) | AQVQLVESGGGLAQPGGSLRLSCAVSGLSLRPLA |
| | CDR2: TESGSTK (SEQ ID NO: 114) | IGWFRQAPGKPRELVAIVTFSGSTKYASSVRGRE |
| | CDR3: SAGRTYGGNTYVA (SEQ ID NO: | TISRDTAENTVYLQMNSLKPEDTAVYQCSAGRTY |
| | 139) | GGNTYVADYWGQGTLVTVSS (SEQ ID NO: |
| | | 57) |
| 163 | CDR1: GLSLRPLA (SEQ ID NO: 93) | AQVQLVESGGGLAQPGGSLRLSCAVSGLSLRPLA |
| | CDR2: TFSGSTR (SEQ ID NO: 113) | IGWFRQAPGKPRELVAIVTESGSTRYASSVRGRF |
| | CDR3: SAGRTYGGNTYVA (SEQ ID NO: | TISRDTAENTVYLQMNSLKPEDTAVYQCSAGRTY |
| | 139) | GGNTYVADYWGQGTLVTVSS |
| | | (SEQ ID NO: 58) |
| 164 | CDR1: EFTFSNYA (SEQ ID NO: 94) | AEVQLVESGGGLVQPGGSLRLSCAASEFTFSNYA |
| | CDR2: NWSGGITT (SEQ ID NO: 115) | MSWVRQAPGKGLEWVSAINWSGGITTYADSVRGR |
| | | FTISRDNAKNTLHLQMNSLKPEDTAVYYCAKGWG |
| | CDR3: AKGWGTTTRDTGL (SEQ ID NO: | TTTRDTGLLSQGTLVTVSS (SEQ ID NO: |
| | 140) | 59) |
| 165 | CDR1: EFTESNYA (SEQ ID NO: 94) | AEVQLVESGGGLVQPGGSLRLSCAASEFTESNYA |
| | CDR2: NWSGGITT (SEQ ID NO: 115) | MSWVRQAPGKGLEWVSAINWSGGITTYADSVKGR |
| | CDR3: ARGWGTTTRDTGL (SEQ ID NO: | FTISRDNAKNTLHLQMNSLKPEDTAVYYCARGWG |
| | 149) | TTTRDTGLLSQGTLVTVSS (SEQ ID NO: |
| | | 60) |
| 166 | CDR1: EFTFSNYA (SEQ ID NO: 94) | AEVQLVESGGGLVQPGGSLRLSCAASEFTESNYA |
| | CDR2: NWSGGITT (SEQ ID NO: 115) | MSWVRQAPGKGLEWVSAINWSGGITTYADSVRGR |
| | CDR3: ARGWGTTTRDTGL (SEQ ID NO: | FTISRDNAKNTLHLQMNSLKPEDTAVYYCARGWG |
| | 149) | TTTRDTGLLSQGTLVTVSS (SEQ ID NO: |
| | | 61) |
| 167 | CDR1: GRADVINV (SEQ ID NO: 97) | AEVQLVESGGGSGQPGGSLSVSCVVSGRADVINV |
| | CDR2: TVGGTY (SEQ ID NO: 116) | MGWYRQAPGKERELVAAITVGGTYYADAVRGRFT |
| | CDR3: NMNEYRVATPTTGQQRFEY(SEQ | ISRDNAKNTLYLQMNSLKPEDTAVYICNMNEYRV |
| | ID NO: 150) | ATPTTGQQRFEYWGQGTQVTVSS |
| | | (SEQ ID NO: 62) |
| 168 | CDR1: GRTENSYFYT (SEQ ID NO: 98) | AQVQLVESGGGLVQAGGSLRLSCAASGRTENSYF |
| | CDR2: ARMNWNADST (SEQ ID NO: | YTMAWFRQAPGKEREFVARMNWNADSTYHADSVR |
| | 107) | GRFTISRENTVYLQMNSLEPEDTAVYYCAAKSIS |
| | CDR3: AAKSISAPKYDY (SEQ ID NO: | APKYDYWGQGTQVTVSS (SEQ ID NO: 63) |
| | 144) | |
| 169 | CDR1: GRTENSYFYT (SEQ ID NO: 98) | AQVQLVESGGGLVQAGGSLRLSCAASGRTENSYF |
| | CDR1: ARMNWNADST (SEQ ID NO: | YTMAWFRQAPGKEREFVARMNWNADSTYHADSVK |
| | 107) | GRFTISRENTVYLQMNSLEPEDTAVYYCAARSIS |
| | CDR1: AARSISAPKYDY (SEQ ID NO: | APKYDYWGQGTQVTVSS (SEQ ID NO: 64) |
| | 151) | |
| 170 | CDR1: GRTENSYFYT (SEQ ID NO: 98) | AQVQLVESGGGLVQAGGSLRLSCAASGRTENSYF |
| | CDR2: ARMNWNADST (SEQ ID NO: | YTMAWFRQAPGKEREFVARMNWNADSTYHADSVR |
| | 107) | GRFTISRENTVYLQMNSLEPEDTAVYYCAARSIS |
| | CDR3: AARSISAPKYDY (SEQ ID NO: | APKYDYWGQGTQVTVSS (SEQ ID NO: 65) |
| | 151) | |
| 171 | CDR1: GRTENSYFYT (SEQ ID NO: 98) | AQVQLVESGGGLVQAGGSLRLSCAASGRTENSYF |
| | CDR2: ARMNWNADST (SEQ ID NO: | YTMAWFRQAPGKEREFVARMNWNADSTYHADSVK |
| | 107) | GRFTISRENTVYLQMNSLEPEDTAVYYCAAKSIS |
| | CDR3: AAKSISAPRYDY (SEQ ID NO: | APRYDYWGQGTQVTVSS (SEQ ID NO: 66) |
| | 152) | |
| 172 | CDR1: GRTFNSYFYT (SEQ ID NO: 98) | AQVQLVESGGGLVQAGGSLRLSCAASGRTENSYF |
| | CDR2: ARMNWNADST (SEQ ID NO: | YTMAWFRQAPGKEREFVARMNWNADSTYHADSVR |
| | 107) | GRFTISRENTVYLQMNSLEPEDTAVYYCAAKSIS |
| | CDR3: AAKSISAPRYDY (SEQ ID NO: | APRYDYWGQGTQVTVSS (SEQ ID NO: 67) |
| | 152) | |
| 173 | CDR1: GRTFNSYFYT (SEQ ID NO: 98) | AQVQLVESGGGLVQAGGSLRLSCAASGRTENSYF |
| | CDR2: ARMNWNADST (SEQ ID NO: | YTMAWFRQAPGKEREFVARMNWNADSTYHADSVK |
| | 107) | GRFTISRENTVYLQMNSLPEDTAVYYCAARSISA |
| | CDR3: AARSISAPRYDY (SEQ ID NO: | PRYDYWGQGTQVTVSS (SEQ ID NO: 68) |
| | 153) | |
| 174 | CDR1: GRTENSYFYT (SEQ ID NO: 98) | AQVQLVESGGGLVQAGGSLRLSCAASGRTENSYF |
| | CDR2: ARMNWNADST (SEQ ID NO: | YTMAWFRQAPGKEREFVARMNWNADSTYHADSVR |
| | 107) | GRFTISRENTVYLQMNSLEPEDTAVYYCAARSIS |
| | CDR3: AARSISAPRYDY (SEQ ID NO: | APRYDYWGQGTQVTVSS (SEQ ID NO: 69) |
| | 153) | |
| 175 | CDR1: EFTSDNFA (SEQ ID NO: 89) | AQVQLVESGGGLVQPGGSLRLSCAASEFTSDNFA |
| | CDR2: SSDGRMRN (SEQ ID NO: 112) | IGWFRQAPGKEREGVSCISSDGRMRNYADSVKGR |
| | CDR3: AAARVGAEYVCYEAYSAWYDH(SEQ | LTISRDNADKTVYLQMNSLKPEDTATYYCAAARV |
| | ID NO: 157) | GAEYVCYEAYSAWYDHWGQGTQVTVSS(SEQ |
| | | ID NO: 70) |
| 176 | CDR1: EFTSDNFA (SEQ ID NO: 89) | AQVQLVESGGGLVQPGGSLRLSCAASEFTSDNFA |
| | CDR2: SSDGRMKN (SEQ ID NO: 108) | IGWFRQAPGKEREGVSCISSDGRMKNYADSVRGR |
| | CDR3: AAARVGAEYVCYEAYSAWYDH | LTISRDNADKTVYLQMNSLKPEDTATYYCAAARV |
| | (SEQ ID NO: 157) | GAEYVCYEAYSAWYDHWGQGTQVTVSS(SEQ |
| | | ID NO: 71) |
| 177 | CDR1: EFTSDNFA (SEQ ID NO: 89) | AQVQLVESGGGLVQPGGSLRLSCAASEFTSDNFA |
| | CDR2: SSDGRMRN (SEQ ID NO: 112) | IGWFRQAPGKEREGVSCISSDGRMRNYADSVRGR |
| | CDR3: AAARVGAEYVCYEAYSAWYDH(SEQ | LTISRDNADKTVYLQMNSLKPEDTATYYCAAARV |
| | ID NO: 157) | GAEYVCYEAYSAWYDHWGQGTQVTVSS(SEQ |
| | | ID NO: 72) |
| 178 | CDR1: GRTFSSYH (SEQ ID NO: 99) | AQVQLVESGGGLVQAGGSPRLSCAASGRTESSYH |
| | CDR2: TWSGGSTY (SEQ ID NO: 109) | MGWFRQAPRKEREFVAAITWSGGSTYYADFVRGR |
| | CDR3: AVGKDFYGIIMRGEYDY (SEQ ID | FTISRDNAKNTVYLQMNSLKPEDTAVYYCAVGKD |
| | NO: 132) | FYGIIMRGEYDYWGQGTLVTVSS (SEQ ID |
| | | NO: 73) |
| 179 | CDR1: GRTFSSYH (SEQ ID NO: 99) | AQVQLVESGGGLVQAGGSPRLSCAASGRTFSSYH |
| | CDR2: TWSGGSTY (SEQ ID NO: 109) | MGWFRQAPRKEREFVAAITWSGGSTYYADFVKGR |
| | CDR3: AVGRDFYGIIMRGEYDY (SEQ ID | FTISRDNAKNTVYLQMNSLKPEDTAVYYCAVGRD |
| | NO: 148) | FYGIIMRGEYDYWGQGTLVTVSS (SEQ ID |
| | | NO: 74) |
| 180 | CDR1: GRTFSSYH (SEQ ID NO: 99) | AQVQLVESGGGLVQAGGSPRLSCAASGRTESSYH |
| | CDR2: TWSGGSTY (SEQ ID NO: 109) | MGWFRQAPRKEREFVAAITWSGGSTYYADFVRGR |
| | CDR3: AVGRDFYGIIMRGEYDY (SEQ ID | FTISRDNAKNTVYLQMNSLKPEDTAVYYCAVGRD |
| | NO: 148) | FYGIIMRGEYDYWGQGTLVTVSS (SEQ ID |
| | | NO: 75) |
| 181 | CDR1: GSIFGFNV (SEQ ID NO: 100) | AEVQLVESGGGLVQAGGSLRLSCAASGSIFGENV |
| | CDR2: GIGTSAT (SEQ ID NO: 110) | MEWYRQAPGKQRELVAVLGIGTSATYADSVRGRF |
| | CDR3: NAKFVY (SEQ ID NO: 133) | TISRDNAKNTVYLQMNSLKPEDTAVYYCNAKFVY |
| | | WGQGTLVTVSS (SEQ ID NO: 76) |
| 182 | CDR1: GSIFGENV (SEQ ID NO: 100) | AEVQLVESGGGLVQAGGSLRLSCAASGSIFGENV |
| | CDR2: GIGTSAT (SEQ ID NO: 110) | MEWYRQAPGKQRELVAVLGIGTSATYADSVKGRF |
| | CDR3: NARFVY (SEQ ID NO: 154) | TISRDNAKNTVYLQMNSLKPEDTAVYYCNARFVY |
| | | WGQGTLVTVSS (SEQ ID NO: 77) |
| 183 | CDR1: GSIFGENV (SEQ ID NO: 100) | AEVQLVESGGGLVQAGGSLRLSCAASGSIFGENV |
| | CDR2: GIGTSAT (SEQ ID NO: 110) | MEWYRQAPGKQRELVAVLGIGTSATYADSVRGRF |
| | CDR3: NARFVY (SEQ ID NO: 154) | TISRDNAKNTVYLQMNSLKPEDTAVYYCNARFVY |
| | | WGQGTLVTVSS (SEQ ID NO: 78) |
| 184 | CDR1: GLGFSALA (SEQ ID NO: 86) | AQVQLVESGGGSVQLGGSLRLSCAVSGLGFSALA |
| | CDR2: SRTGGTTY (SEQ ID NO: 106) | MGWFRQAPGKERERVAAISRTGGTTYYTDSVRGR |
| | CDR3: ASRYTALLSTTARKYDY (SEQ ID | FTISRDNGKNMVYLQMNSLKPEDAAVYYCASRYT |
| | NO: 134) | ALLSTTARKYDYWGQGTQVTVSS (SEQ ID |
| | | NO: 79) |
| 185 | CDR1: GLGFSALA (SEQ ID NO: 86) | AQVQLVESGGGSVQLGGSLRLSCAVSGLGFSALA |
| | CDR2: SRTGGTTY (SEQ ID NO: 106) | MGWFRQAPGKERERVAAISRTGGTTYYTDSVKGR |
| | | FTISRDNGKNMVYLQMNSLKPEDAAVYYCASRYT |
| | CDR3: ASRYTALLSTTARRYDY (SEQ ID | ALLSTTARRYDYWGQGTQVTVSS (SEQ ID |
| | NO: 155) | NO: 80) |
| 186 | CDR1: GLGFSALA (SEQ ID NO: 86) | AQVQLVESGGGSVQLGGSLRLSCAVSGLGFSALA |
| | CDR2: SRTGGTTY (SEQ ID NO: 106) | MGWFRQAPGKERERVAAISRTGGTTYYTDSVRGR |
| | CDR3: ASRYTALLSTTARRYDY (SEQ ID | FTISRDNGKNMVYLQMNSLKPEDAAVYYCASRYT |
| | NO: 155) | ALLSTTARRYDYWGQGTQVTVSS (SEQ ID |
| | | NO: 81) |
| 187 | CDR1: GITFSINT (SEQ ID NO: 101) | EVQLVESGGGLVQAGGSLRLSCAASGITFSINTM |
| | CDR2: LISSIGDTYYADSVRG (SEQ ID | GWYRQAPGRQRELVALISSIGDTYYADSVRGRFT |
| | NO: 111) | ISRDNARNTVYLQMNSLRPEDTAVYYCRRERTAA |
| | CDR3: RRFRTAAQGTDY (SEQ ID NO: | QGTDYWGQGTQVTVSS (SEQ ID NO: 205) |
| | 136) | |

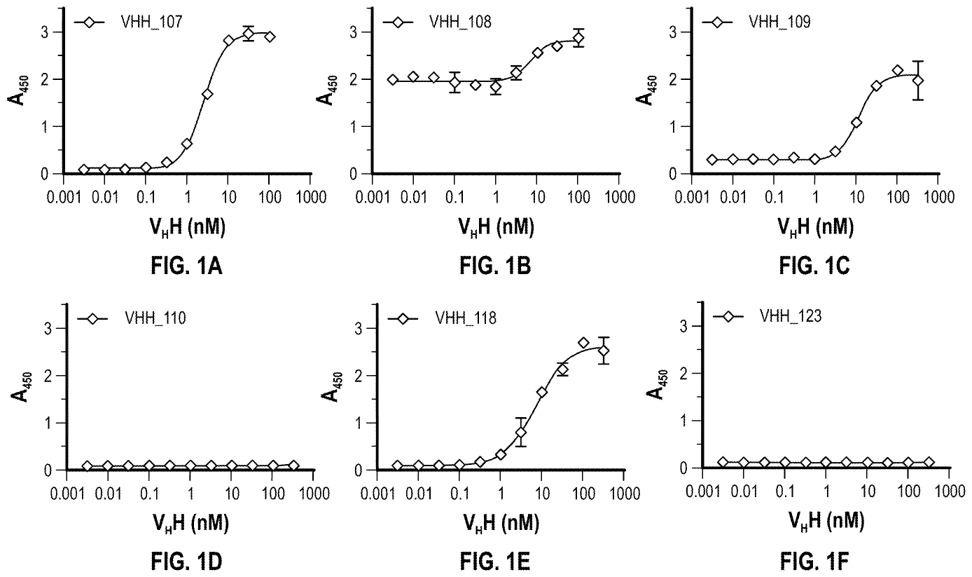

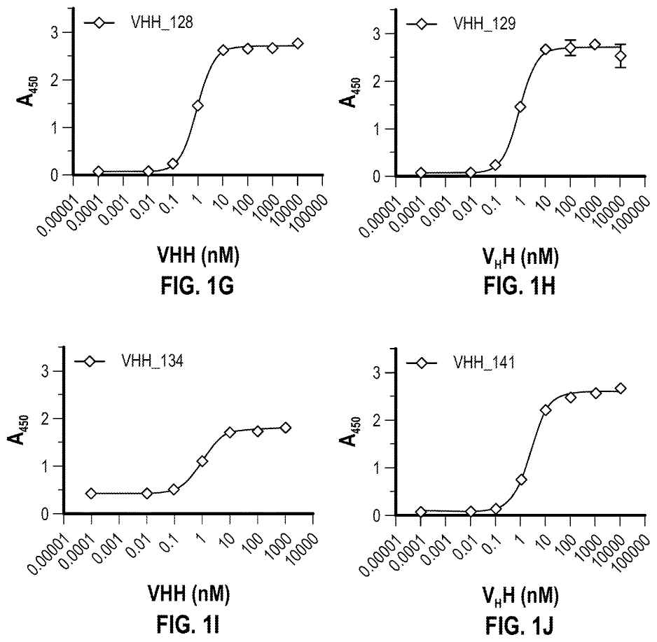

FIGS. 1A-1J present ELISA affinity binding curves of exemplary VHH proteins against the human HER2 receptor, after protein expression and purification.

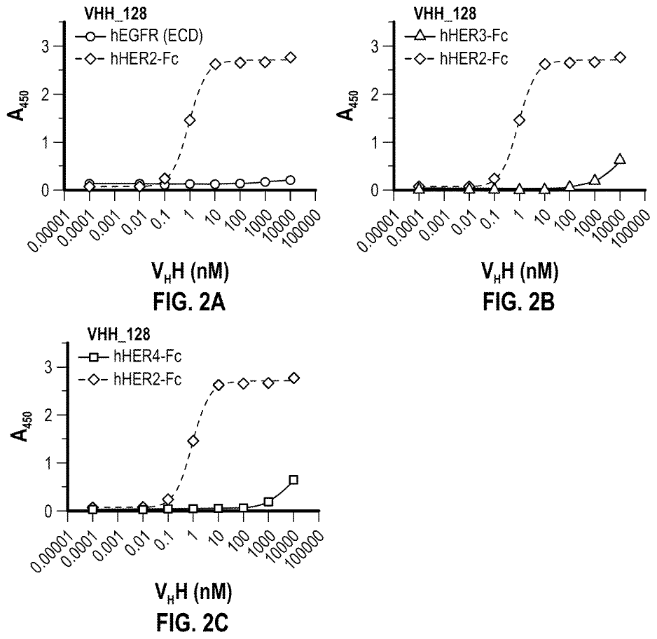

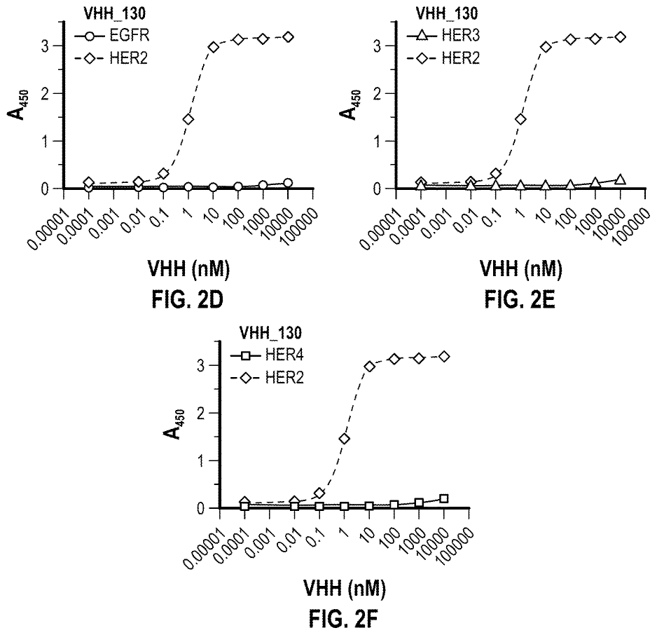

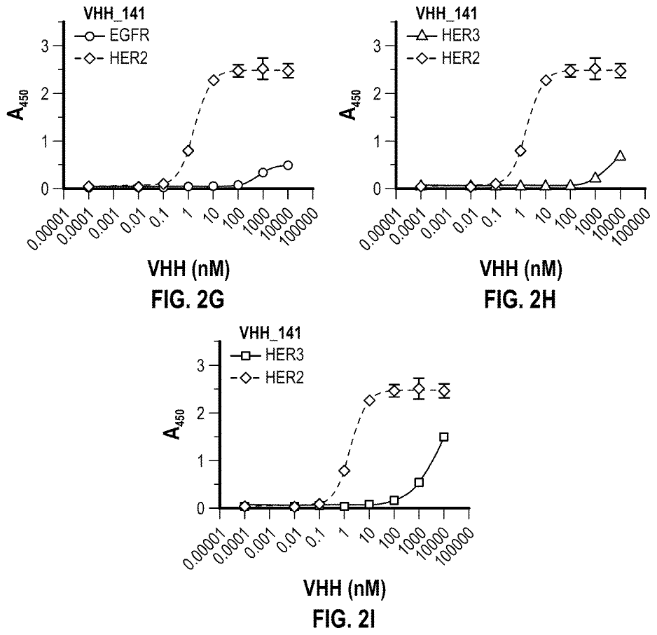

The binding specificity of exemplary VHH proteins to other receptors within the ErbB family is shown in FIGS. 2A-2I.

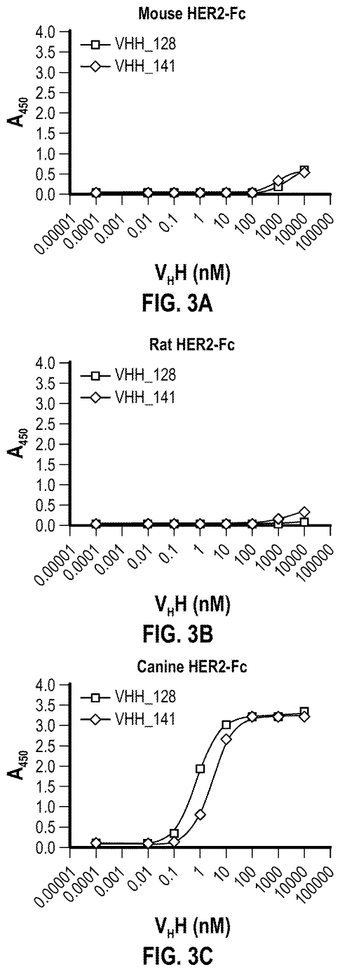

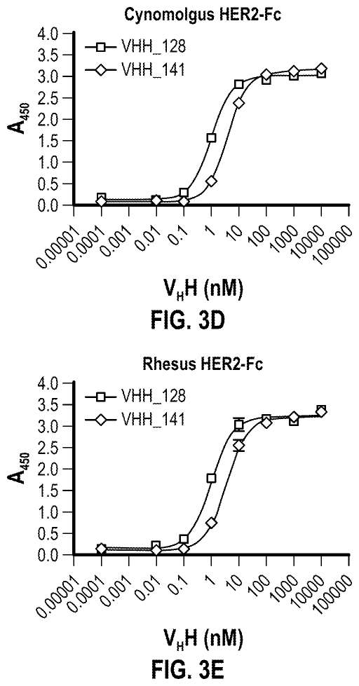

The cross-reactivity of exemplary VHH proteins to cross-species HER2 receptors found in other animals is presented in FIGS. 3A-3E.

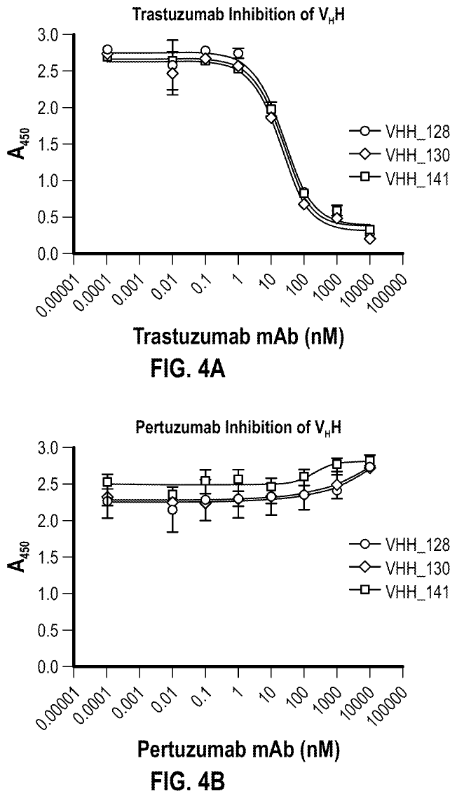

The competitive inhibition of exemplary VHH proteins with trastuzumab and pertuzumab is presented in FIGS. 4A-4B.

### Example 2: Radiolabeling of HER2-Binding VHH Domains and In Vitro Assays

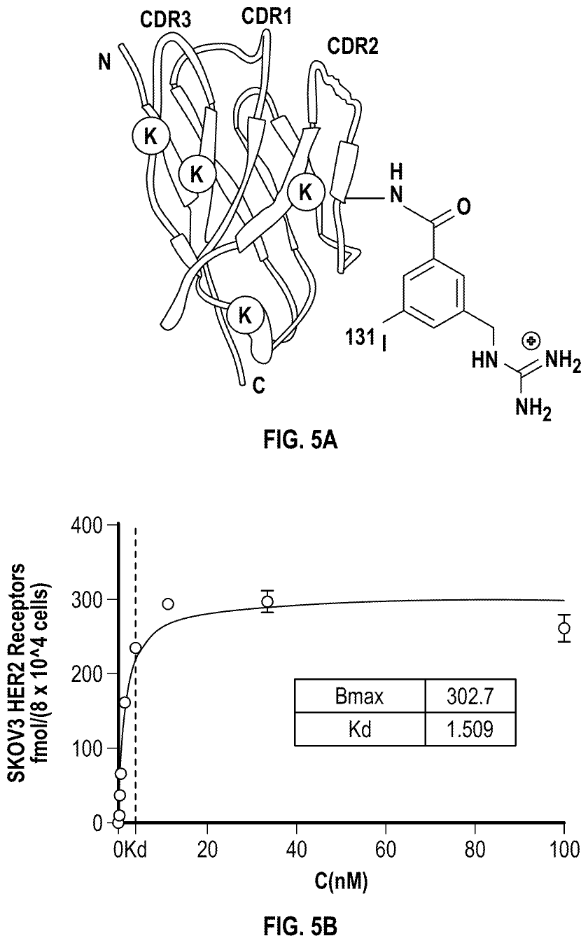

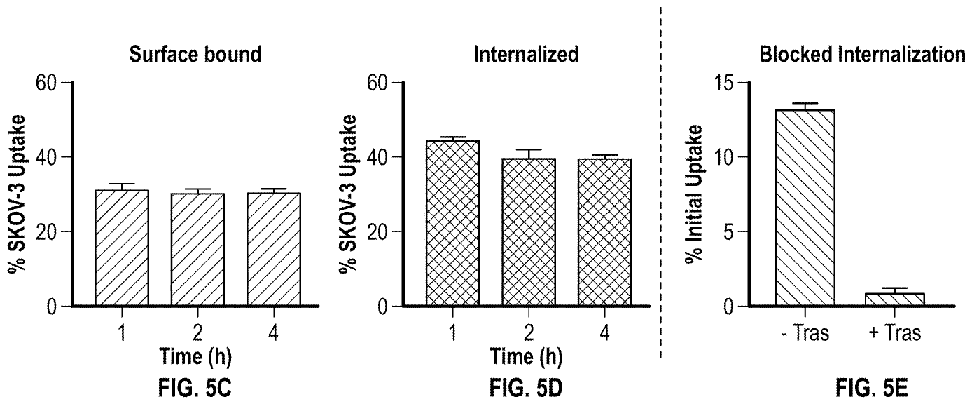

FIG. 5A depicts a structure of VHH radiolabeled with [¹³¹I]-isoSGMIB.

FIGS. 5B-5E present the results of an in vitro cell assay for evaluating VHH_128 HER2 binding after being radiolabeled with [¹³¹I]-isoSGMIB. (B) Affinity to SKOV-3 ovarian cancer cells. Levels of [¹³¹I]-isoSGMIB-VHH128 that are (C) surface bound or (D) internalized within SKOV-3 cells after a 2-hour incubation. (E) Measure of [131I] internalization in SKOV-3 cells in presence of 100-fold trastuzumab.

### Example 3: In Vivo Studies

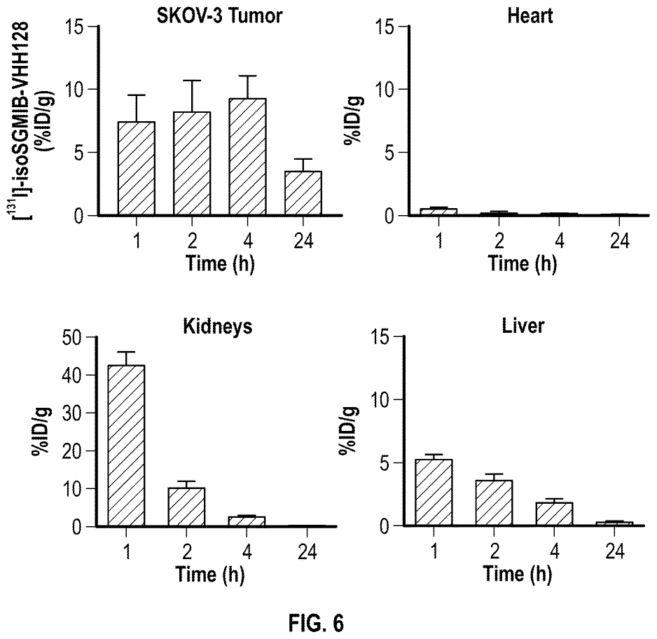

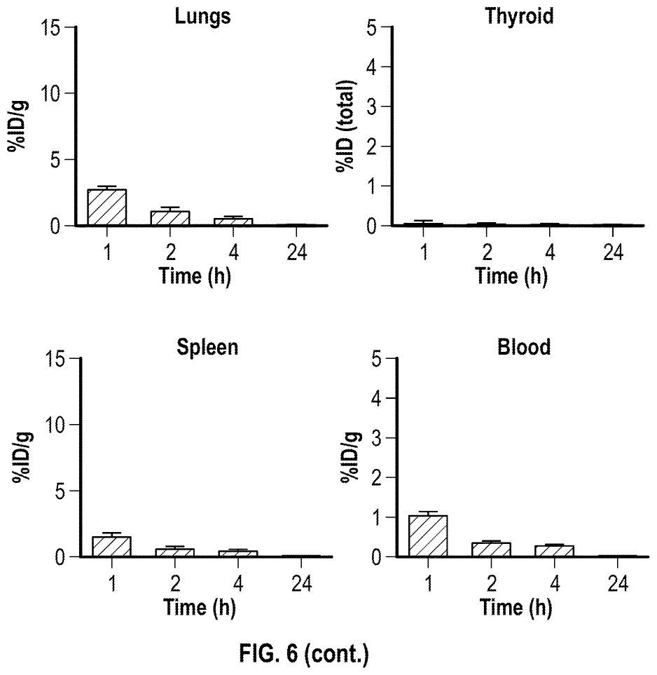

FIG. 6 depicts tissue accumulation levels over 24 hours for [¹³¹I]-isoSGMIB-VHH128 in a SKOV-3 xenograft tumor model in female athymic mice.

Table 3 presents a complete biodistribution accumulation tissue panel for [¹³¹I]-isoSGMIB-VHH128 in a SKOV-3 tumor xenograft in female athymic mice. Injected dose consisted of 5 μCi prepared at a specific activity of 1 μCi/ng and intravenously administered in 100 μL via the tail vein.

TABLE 3

| | Tissue Levels (% IA/g) | | | | | |
|---|---|---|---|---|---|---|
| | 1 h | 2 h | 4 h | 8 h | 16 h | 24 h |
| Liver | 1.04 + 0.12 | 0.76 + 0.05 | 0.22 + 0.03 | 0.09 + 0.01 | 0.04 + 0.02 | 0.03 + 0.01 |
| Spleen | 0.44 + 0.15 | 0.49 + 0.12 | 0.13 + 0.04 | 0.05 + 0.01 | 0.04 + 0.01 | 0.04 + 0.01 |
| Lungs | 1.22 + 0.46 | 0.88 + 0.14 | 0.27 + 0.07 | 0.18 + 0.07 | 0.10 + 0.03 | 0.09 + 0.03 |
| Heart | 1.51 + 2.30 | 0.26 + 0.02 | 0.10 + 0.02 | 0.04 + 0.01 | 0.04 + 0.01 | 0.02 + 0.01 |
| Kidneys | 35.63 + 10.98 | 10.03 + 2.67 | 2.43 + 0.69 | 0.47 + 0.09 | 0.19 + 0.03 | 0.11 + 0.02 |
| Bladder | 1.70 + 0.84 | 0.57 + 0.34 | 0.21 + 0.05 | 0.05 + 0.05 | 0.08 + 0.02 | 0.04 + 0.05 |
| Stomach | 1.00 + 0.18 | 0.98 + 0.21 | 0.19 + 0.06 | 0.17 + 0.09 | 0.03 + 0.02 | 0.02 + 0.01 |
| Sm. Int. | 0.72 + 0.21 | 0.83 + 0.16 | 0.21 + 0.04 | 0.10 + 0.03 | 0.03 + 0.01 | 0.02 + 0.01 |
| Lg. Int. | 0.31 + 0.11 | 0.27 + 0.10 | 0.98 + 0.33 | 0.45 + 0.25 | 0.08 + 0.02 | 0.05 + 0.01 |
| Thyroid | 0.44 + 0.14 | 1.03 + 0.24 | 0.40 + 0.09 | 0.28 + 0.07 | 0.89 + 0.24 | 0.83 + 0.74 |
| Muscle | 0.30 + 0.06 | 0.11 + 0.02 | 0.04 + 0.02 | 0.02 + 0.01 | 0.02 + 0.01 | 0.01 + 0.00 |
| Blood | 0.70 + 0.24 | 0.39 + 0.02 | 0.11 + 0.02 | 0.07 + 0.01 | 0.06 + 0.01 | 0.04 + 0.01 |
| Urine | 472.47 + 500.42 | 295.30 + 124.00 | 17.54 + 14.73 | 3.25 + 2.15 | 1.38 + 1.18 | 0.19 + 0.13 |
| Tumor | 5.58 + 1.90 | 4.83 + 0.98 | 6.97 + 2.61 | 4.67 + 2.51 | 6.53 + 1.18 | 3.11 + 1.25 |
| Bone | 0.63 + 0.17 | 0.16 + 0.04 | 0.09 + 0.02 | 0.01 + 0.02 | 0.02 + 0.01 | 0.01 + 0.01 |
| Skin | 1.73 + 1.07 | 0.88 + 0.92 | 0.22 + 0.09 | 0.12 + 0.04 | 0.08 + 0.03 | 0.04 + 0.01 |
| Brain | 0.04 + 0.00 | 0.02 + 0.00 | 0.01 + 0.00 | 0.00 + 0.00 | 0.00 + 0.00 | 0.00 + 0.00 |
| Tail | 1.30 + 0.45 | 0.47 + 0.28 | 0.24 + 0.09 | 0.21 + 0.14 | 0.06 + 0.03 | 0.04 + 0.02 |

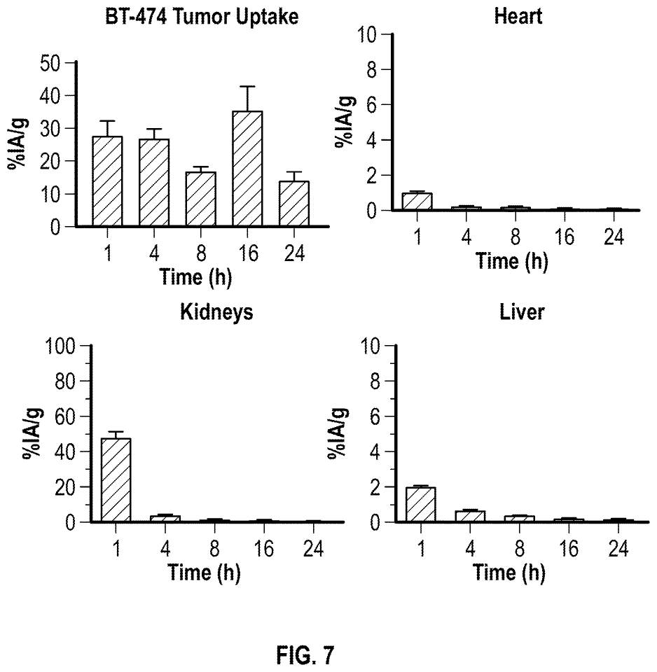

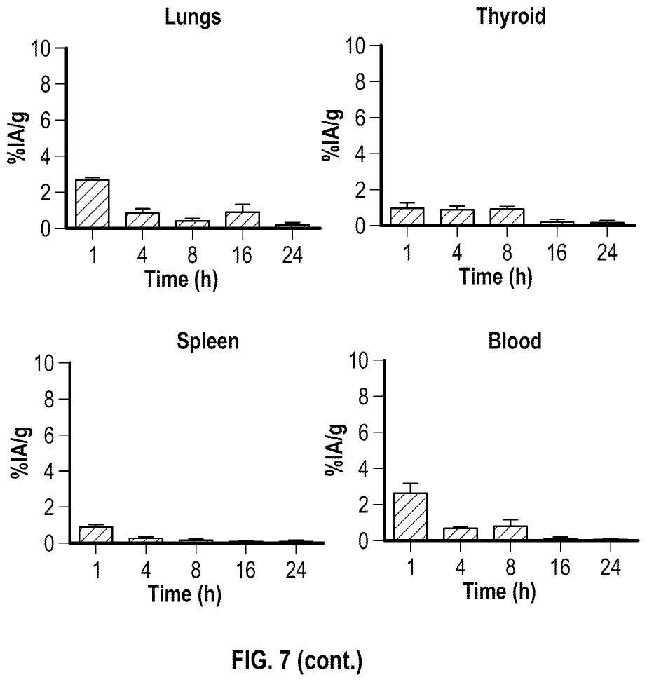

FIG. 7 depicts tissue accumulation levels over 24 hours for [¹³¹I]-isoSGMIB-VHH128 in a BT-474 xenograft tumor model in female NOD SCID mice.

Table 4 presents a complete biodistribution accumulation tissue panel for [¹³¹I]-isoSGMIB-VHH128 in a BT-474 tumor model in female NOD SCID mice. Injected dose consisted of 5 μCi prepared at a specific activity of 1 μCi/ng and intravenously administered in 100 μL via the tail vein.

TABLE 4

| | Tissue Levels (% IA/g) | | | | | |
|---|---|---|---|---|---|---|
| | 1 h | 2 h | 4 h | 8 h | 16 h | 24 h |
| Liver | 1.90 + 0.21 | 0.76 + 0.05 | 0.74 + 0.41 | 0.31 + 0.17 | 0.10 + 0.02 | 0.06 + 0.02 |
| Spleen | 0.89 + 0.18 | 0.49 + 0.12 | 0.67 + 0.84 | 0.18 + 0.08 | 0.04 + 0.02 | 0.08 + 0.11 |
| Lungs | 2.66 + 0.28 | 0.88 + 0.14 | 1.19 + 0.78 | 0.49 + 0.20 | 0.88 + 0.87 | 0.19 + 0.13 |
| Heart | 1.02 + 0.17 | 0.26 + 0.02 | 0.34 + 0.23 | 0.19 + 0.07 | 0.05 + 0.01 | 0.03 + 0.01 |
| Kidneys | 48.26 + 6.91 | 10.03 + 2.67 | 5.58 + 4.12 | 2.95 + 3.68 | 0.75 + 0.73 | 0.22 + 0.12 |
| Bladder | 7.40 + 2.37 | 0.57 + 0.34 | 11.14 + 6.06 | 4.09 + 3.12 | 0.67 + 0.28 | 0.33 + 0.29 |
| Stomach | 1.31 + 0.55 | 0.96 + 0.21 | 2.73 + 0.31 | 1.43 + 0.77 | 0.47 + 0.53 | 0.16 + 0.08 |
| Sm. Int. | 1.00 + 0.28 | 0.83 + 0.16 | 2.21 + 0.74 | 0.89 + 0.37 | 0.56 + 0.32 | 0.16 + 0.11 |
| Lg. Int. | 0.51 + 0.18 | 0.27 + 0.10 | 4.14 + 3.00 | 4.11 + 2.12 | 3.23 + 1.63 | 1.22 + 0.78 |
| Thyroid | 1.08 + 0.51 | 1.03 + 0.24 | 1.12 + 0.35 | 1.03 + 0.13 | 0.29 + 0.21 | 0.18 + 0.07 |
| Muscle | 0.44 + 0.11 | 0.11 + 0.02 | 0.25 + 0.11 | 0.16 + 0.09 | 0.05 + 0.02 | 0.04 + 0.03 |
| Blood | 2.64 + 1.10 | 0.39 + 0.02 | 0.75 + 0.21 | 0.75 + 0.64 | 0.09 + 0.07 | 0.03 + 0.01 |
| Urine | 307.35 + 123.64 | 295.30 + 124.00 | 75.22 + 21.01 | 48.19 + 19.80 | 8.34 + 4.41 | 3.18 + 2.09 |
| Tumor | 27.56 + 9.99 | 4.83 + 0.98 | 34.57 + 18.24 | 27.84 + 24.96 | 36.14 + 16.92 | 14.02 + 6.02 |
| Bone | 0.65 + 0.20 | 0.16 + 0.04 | 0.44 + 0.21 | 0.12 + 0.05 | 0.06 + 0.02 | 0.07 + 0.03 |
| Skin | 2.16 + 0.67 | 0.88 + 0.92 | 1.31 + 0.63 | 0.37 + 0.14 | 0.38 + 0.16 | 0.22 + 0.13 |
| Brain | 0.08 + 0.02 | 0.02 + 0.00 | 0.05 + 0.02 | 0.02 + 0.01 | 0.00 + 0.00 | 0.00 + 0.00 |
| Tail | 3.45 + 1.92 | 0.47 + 0.28 | 2.22 + 0.78 | 1.69 + 0.68 | 0.63 + 0.24 | 0.43 + 0.15 |

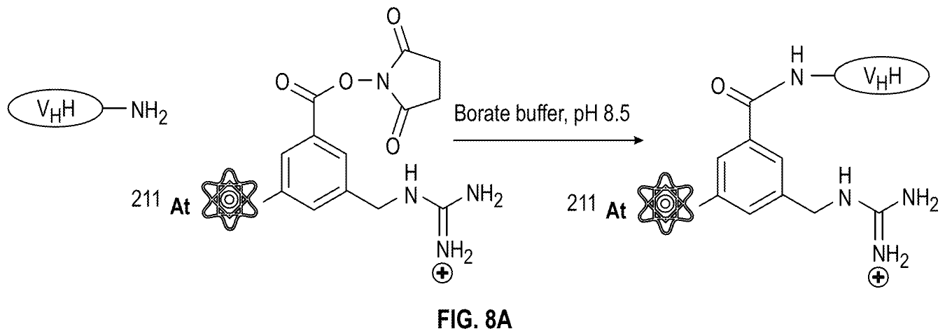

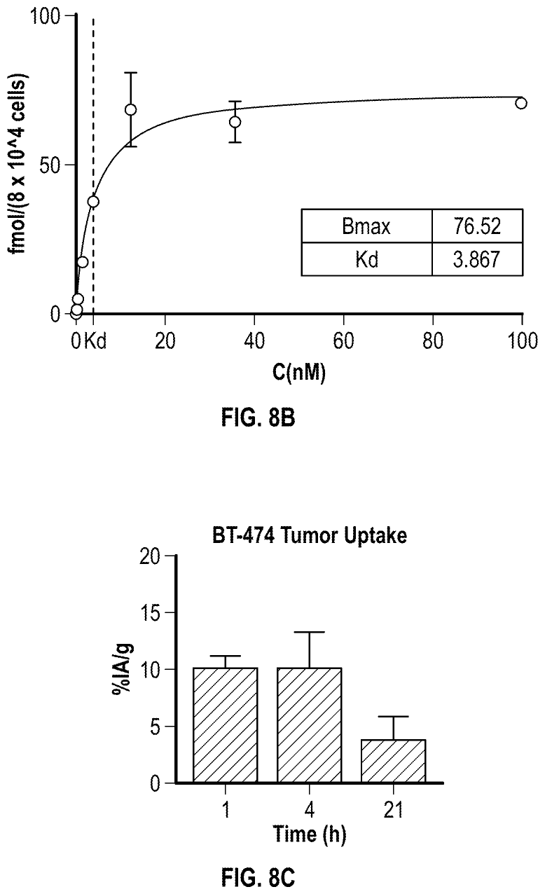

FIGS. 8A-8C depict HER2 targeted alpha-particle therapy utilizing ²¹¹Astatine labeled VHH. (A) Scheme and structure for labeling VHH_128 with ²¹¹At using isoSAGMB. (B) Affinity binding curve for [²¹¹At]-isoSAGMB-VHH128 against the HER2-expressing BT-474 cells. (C) Accumulation levels in BT-474 tumors xenografted in athymic mice.

Table 5 presents a complete biodistribution accumulation tissue panel for [²¹¹At]-isoSAGMB-VHH1028 in a BT-474 tumor model in female athymic mice. Injected dose consisted of 5 μCi prepared at a specific activity of 1 μCi/ng and intravenously administered in 100 μL via the tail vein.

TABLE 5

| | 1 h | | 4 h | | 21 h | |
|---|---|---|---|---|---|---|
| | mean | SD | mean | SD | mean | SD |
| Liver | 5.48 | 1.42 | 1.96 | 0.25 | 0.43 | 0.19 |
| Spleen | 4.24 | 1.82 | 3.31 | 1.23 | 0.51 | 0.29 |
| Lungs | 4.50 | 2.61 | 2.15 | 1.38 | 0.72 | 0.31 |
| Heart | 1.67 | 0.72 | 1.15 | 0.50 | 0.24 | 0.12 |
| Kidneys | 34.20 | 13.63 | 4.46 | 0.62 | 0.57 | 0.23 |
| Bladder | 8.87 | 7.43 | 5.42 | 4.53 | 0.69 | 0.35 |
| Stomach | 5.61 | 2.51 | 7.61 | 2.77 | 1.49 | 0.87 |
| Sm. Int. | 2.24 | 1.29 | 1.28 | 0.28 | 0.24 | 0.08 |
| Lg. Int. | 0.77 | 0.54 | 2.98 | 0.93 | 0.33 | 0.12 |
| Thyroid | 2.20 | 0.90 | 2.41 | 1.16 | 2.02 | 0.93 |
| Muscle | 0.53 | 0.04 | 0.27 | 0.07 | 0.09 | 0.04 |
| Blood | 1.41 | 0.89 | 0.58 | 0.13 | 0.14 | 0.05 |
| Urine | 215.10 | 93.55 | 80.92 | 53.50 | 3.56 | 1.18 |
| Tumor | 10.28 | 0.90 | 10.21 | 3.10 | 4.00 | 1.90 |
| Bone | 0.92 | 0.53 | 0.52 | 0.21 | 0.13 | 0.05 |
| Skin | 3.72 | 2.46 | 1.97 | 0.88 | 0.51 | 0.20 |
| Brain | 0.15 | 0.04 | 0.10 | 0.03 | 0.03 | 0.01 |
| Tail | 2.83 | 3.22 | 0.80 | 0.24 | 0.29 | 0.27 |

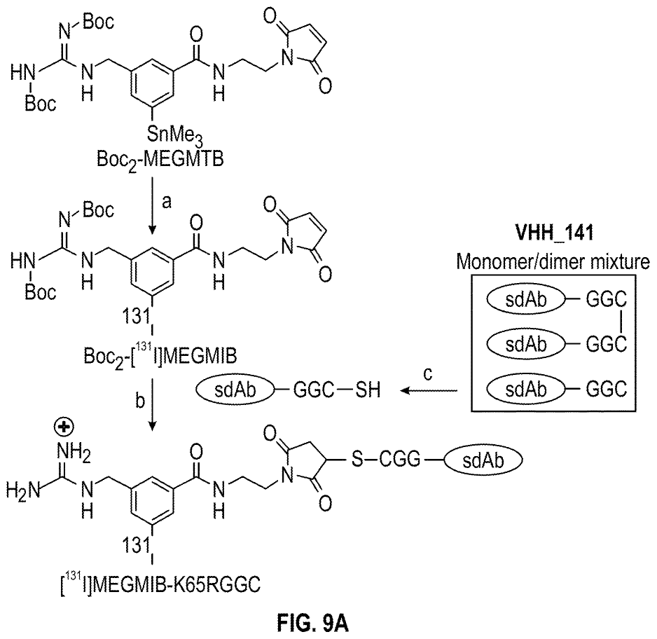

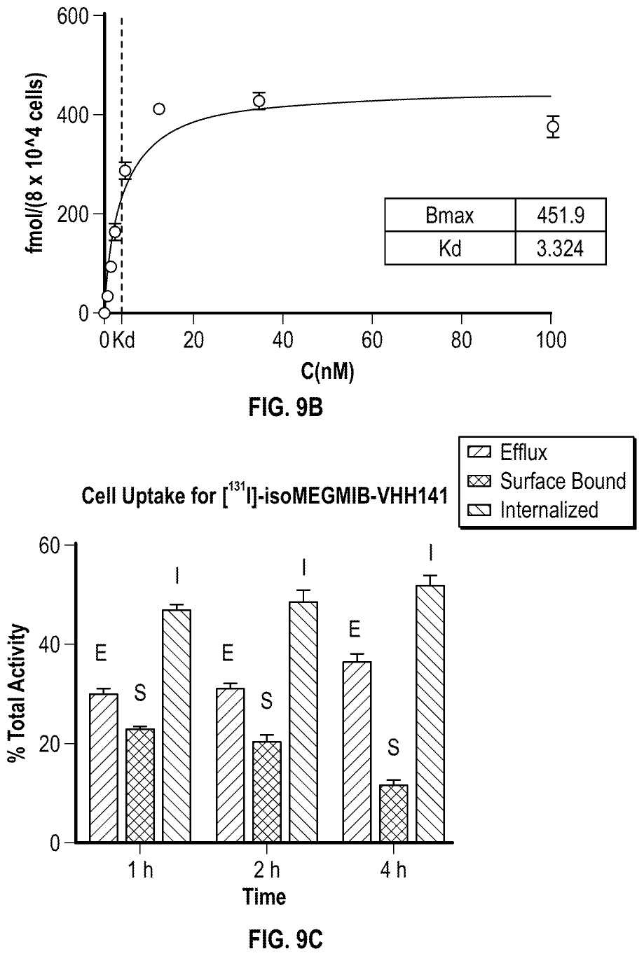

FIGS. 9A-9C depict radiolabeling of HER2-targeted VHH using maleimide-based radiolabeling chemistry. (A) Scheme and structure for labeling VHH_1041 with ¹³¹I using isoMEGMIB. (B) Affinity binding curve for [131I]-isoMEGMIB-VHH141 against the HER2-expressing BT-474 cells. (C) In vitro cellular binding and internalization ratios on BT-474 cells.

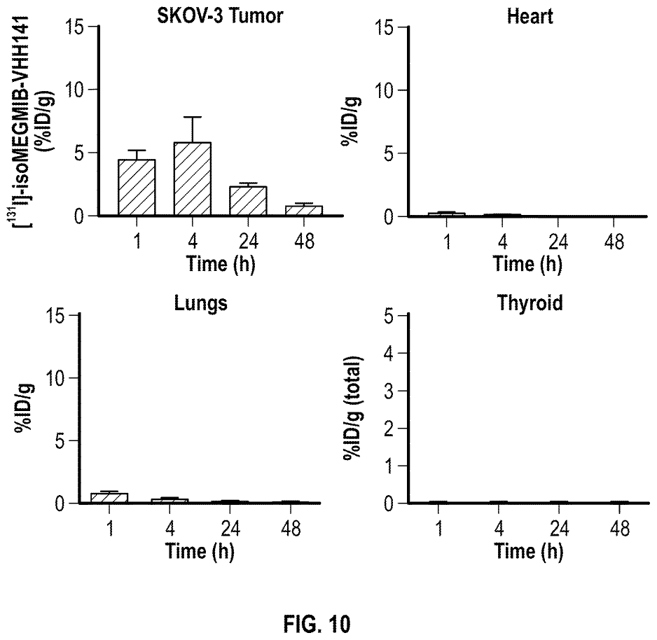

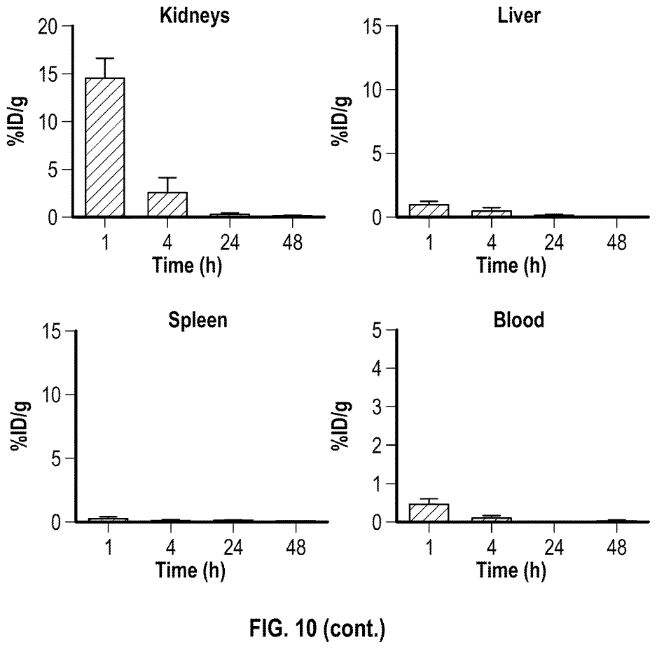

FIG. 10 depicts tissue accumulation levels over 48 hours for [¹³¹I]-isoMEGMIB-VHH141 in a SKOV-3 xenograft tumors in female athymic mice.

Table 6 presents a complete biodistribution accumulation tissue panel for [131]-isoMEGMIB-VHH141 in a SKOV-3 tumor xenograft in female athymic mice. Injected dose consisted of 5 μCi prepared at a specific activity of 1 μCi/ng and intravenously administered in 100 μL via the tail vein.

TABLE 6

| | Tissue Levels (% IA/g) | | | |
|---|---|---|---|---|
| | 1 h | 4 h | 24 h | 48 h |
| Liver | 1.04 + 0.37 | 0.56 + 0.42 | 0.04 + 0.01 | 0.02 + 0.01 |
| Spleen | 0.30 + 0.10 | 0.07 + 0.04 | 0.02 + 0.01 | 0.02 + 0.00 |
| Lungs | 0.92 + 0.18 | 0.40 + 0.17 | 0.10 + 0.04 | 0.12 + 0.07 |
| Heart | 0.36 + 0.16 | 0.08 + 0.07 | 0.01 + 0.00 | 0.01 + 0.01 |
| Kidneys | 14.64 + 4.61 | 2.68 + 3.33 | 0.29 + 0.06 | 0.11 + 0.04 |
| Bladder | 1.57 + 0.68 | 0.31 + 0.26 | 0.01 + 0.02 | 0.03 + 0.01 |
| Stomach | 0.60 + 0.22 | 0.20 + 0.15 | 0.01 + 0.00 | 0.01 + 0.00 |
| Sm. Int. | 1.53 + 0.34 | 0.51 + 0.41 | 0.01 + 0.00 | 0.02 + 0.01 |
| Lg. Int. | 0.39 + 0.19 | 2.54 + 1.20 | 0.02 + 0.01 | 0.04 + 0.02 |
| Thyroid | −0.02 + 0.28 | 0.14 + 0.13 | 0.08 + 0.02 | 0.07 + 0.05 |
| Muscle | 0.45 + 0.50 | 0.04 + 0.02 | 0.01 + 0.00 | 0.01 + 0.00 |
| Blood | 0.49 + 0.24 | 0.11 + 0.09 | 0.03 + 0.00 | 0.02 + 0.01 |
| Urine | 390.40 + 289.10 | 18.91 + 24.31 | 0.10 + 0.04 | 0.32 + 0.26 |
| Tumor | 4.51 + 1.49 | 5.93 + 4.30 | 2.40 + 0.40 | 0.86 + 0.30 |
| Bone | 1.36 + 1.54 | 0.05 + 0.02 | 0.01 + 0.01 | 0.01 + 0.01 |
| Skin | 3.01 + 3.63 | 0.22 + 0.13 | 0.04 + 0.02 | 0.03 + 0.02 |
| Brain | 0.08 + 0.10 | 0.01 + 0.01 | 0.00 + 0.00 | 0.00 + 0.00 |
| Tail | 1.24 + 0.37 | 0.30 + 0.13 | 0.04 + 0.01 | 0.04 + 0.04 |

1. A HER2-binding agent comprising a HER2-binding VHH domain that comprises:
a) a complementarity determining region (CDR) 1 comprising an amino acid sequence selected from SEQ ID NOs: 82-101; a CDR2 comprising an amino acid sequence selected from SEQ ID NOs: 102-123; and a CDR3 comprising an amino acid sequence selected from SEQ ID NOs: 124-157 and 206;
b) a CDR1, CDR2 and CDR3 comprising the amino acid sequence of a CDR1, CDR2 and CDR3 contained in a VHH sequence selected from SEQ ID NOs: 1-81 and 205;
c) a VHH sequence selected from SEQ ID NOs: 1-81 and 205;
d) an amino acid sequence having at least 80%, 85%, 90%, 95%, 96%, 97%, 98% or 99% sequence identity with a VHH sequence selected from SEQ ID NOs: 1-81 and 205;
e) an amino acid sequence that has 1-25, 1-20, 1-15, 1-10, 1-5, or 1-3 additions, substitutions or deletions compared to a reference VHH sequence selected from SEQ ID NO: 1-81 and 205.

2. The HER2-binding agent of claim 1, wherein the VHH domain comprises:
a) a CDR1 comprising SEQ ID NO: 101; a CDR2 comprising SEQ ID NO: 111; and a CDR3 comprising SEQ ID NO: 135 or 136;
b) a CDR1 comprising SEQ ID NO: 101; a CDR2 comprising SEQ ID NO: 111; and a CDR3 comprising SEQ ID NO: 135;
c) a CDR1 comprising SEQ ID NO: 101; a CDR2 comprising SEQ ID NO: 111; and a CDR3 comprising SEQ ID NO: 136;
d) a CDR1 comprising SEQ ID NO: 82; a CDR2 comprising SEQ ID NO: 102; and a CDR3 comprising SEQ ID NO: 124, 145, 146 or 147;
e) a CDR1 comprising SEQ ID NO: 83; a CDR2 comprising the amino acid sequence of SEQ ID NO: 103; and a CDR3 comprising SEQ ID NO: 125;
f) a CDR1 comprising SEQ ID NO: 84; a CDR2 comprising SEQ ID NO: 104; and a CDR3 comprising SEQ ID NO: 126;
g) a CDR1 comprising SEQ ID NO: 85; a CDR2 comprising SEQ ID NO: 105; and a CDR3 comprising SEQ ID NO: 127;
h) a CDR1 comprising SEQ ID NO: 86; a CDR2 comprising SEQ ID NO: 106; and a CDR3 comprising SEQ ID NO: 128, 130, 134 or 155;
i) a CDR1 comprising SEQ ID NO: 87; a CDR2 comprising the amino acid sequence selected from SEQ ID NO: 117; and a CDR3 comprising SEQ ID NO: 129;
j) a CDR1 comprising SEQ ID NO: 88; a CDR2 comprising SEQ ID NO: 118; and a CDR3 comprising the amino acid sequence selected from SEQ ID NO: 130;
k) a CDR1 comprising SEQ ID NO: 89; a CDR2 comprising SEQ ID NO: 108 or 112; and a CDR3 comprising SEQ ID NO: 131 or 157;
l) a CDR1 comprising SEQ ID NO: 89; a CDR2 comprising SEQ ID NO: 108; and a CDR3 comprising SEQ ID NO: 131;
m) a CDR1 comprising SEQ ID NO: 89; a CDR2 comprising SEQ ID NO: 108 or 112; and a CDR3 comprising SEQ ID NO: 156;
n) a CDR1 comprising SEQ ID NO: 89; a CDR2 comprising SEQ ID NO: 108 or 112; and a CDR3 comprising SEQ ID NO: 157;
o) a CDR1 comprising SEQ ID NO: 90; a CDR2 comprising SEQ ID NO: 109 and a CDR3 comprising SEQ ID NO: 132;
p) a CDR1 comprising SEQ ID NO: 91; a CDR2 comprising SEQ ID NO: 119 and a CDR3 comprising SEQ ID NO: 137;
q) a CDR1 comprising SEQ ID NO: 92; a CDR2 comprising SEQ ID NO: 120 and a CDR3 comprising SEQ ID NO: 138;
r) a CDR1 comprising SEQ ID NO: 93; a CDR2 comprising SEQ ID NO: 113 or 114 and a CDR3 comprising SEQ ID NO: 139;
s) a CDR1 comprising SEQ ID NO: 93; a CDR2 comprising SEQ ID NO: 114 and a CDR3 comprising SEQ ID NO: 206;
t) a CDR1 comprising SEQ ID NO: 94; a CDR2 comprising SEQ ID NO: 115 and a CDR3 comprising SEQ ID NO: 140 or 149;
u) a CDR1 comprising SEQ ID NO: 95; a CDR2 comprising SEQ ID NO: 121 and a CDR3 comprising SEQ ID NO: 141;
v) a CDR1 comprising SEQ ID NO: 96; a CDR2 comprising SEQ ID NO: 122; and a CDR3 comprising SEQ ID NO: 142;
w) a CDR1 comprising SEQ ID NO: 97; a CDR2 comprising SEQ ID NO: 116; and a CDR3 comprising SEQ ID NO: 150;
x) a CDR1 comprising SEQ ID NO: 97; a CDR2 comprising SEQ ID NO: 123; and a CDR3 comprising SEQ ID NO: 143;
y) a CDR1 comprising SEQ ID NO: 98; a CDR2 comprising SEQ ID NO: 107; and a CDR3 comprising the amino acid sequence selected from SEQ ID NO: 144, 151, 152 and 153;
z) a CDR1 comprising SEQ ID NO: 99; a CDR2 comprising SEQ ID NO: 109; and a CDR3 comprising SEQ ID NO: 132 or 148;
aa) a CDR1 comprising SEQ ID NO: 100; a CDR2 comprising SEQ ID NO: 110; and a CDR3 comprising SEQ ID NO: 133 or 154; or
bb) a CDR1 comprising SEQ ID NO: 101; a CDR2 comprising SEQ ID NO: 111; and a CDR3 comprising SEQ ID NO: 136.

3. The HER2-binding agent of claim 1 or 2, wherein said HER2-binding agent is monovalent.

4. The HER2-binding agent of claim 1 or 2, wherein said HER2-binding agent is multivalent, optionally wherein said HER2-binding agent is bivalent, trivalent, or tetravalent.

5. The HER2-binding agent of any one of claims 1-4, wherein said HER2-binding agent is monospecific.

6. The HER2-binding agent of any one of claims 1, 2, or 4, wherein said HER2-binding agent is multispecific and specifically binds HER2 and a different antigen.

7. The HER2-binding agent of any one of claims 1-6, wherein the VHH domain is humanized.

8. The HER2-binding agent of any one of claims 1-7, wherein said HER2-binding agent comprises a fusion protein comprising the HER2 binding VHH domain and a heterologous sequence.

9. The HER2-binding agent of any one of claims 1-8, wherein said HER2-binding agent comprises an N-terminal amino acid residue or polypeptide sequence, optionally wherein the N-terminal residue or sequence improves protein expression, solubility, purification, half-life, or avidity, optionally wherein the N-terminal residue or sequence is a Met-Ala or an Ala leader peptide.

10. The HER2-binding agent of any one of claims 1-9, wherein said HER2-binding agent comprises a C-terminal amino acid residue or polypeptide sequence, optionally wherein the C-terminal residue or sequence facilitates radiolabeling selectivity, loading, cell internalization, half-life, or avidity.

11. The HER2-binding agent of any one of claims 1-10, wherein said HER2-binding agent comprises a polypeptide sequence having the structure:
VHH-(GlyₙXaaₘGlyₖ)ⱼ
i) where VHH is selected from SEQ ID NOs: 1-81 and 205,
ii) n=0-5,
iii) m=0-5,
iv) k=0-6,
v) j=0-8, and
vi) Xaa is an amino acid that enables site conjugation to radiolabeled prosthetic groups, or direct radiolabeling.

12. The HER2-binding agent of any one of claims 8-11, wherein the fusion protein comprises an Fc region.

13. The HER2-binding agent of any one of claims 8-11, wherein the fusion protein is a chimeric antigen receptor (CAR).

14. Isolated nucleic acid(s) encoding the HER2-binding agent of any one of claims 1-13.

15. Vector(s) comprising the nucleic acid(s) of claim 14, optionally wherein the vector(s) are expression vector(s).

16. A host cell comprising the nucleic acid(s) of claim 14 or the vector(s) of claim 15, optionally wherein the host cell is a eukaryotic cell or a prokaryotic cell, optionally wherein the eukaryotic cell is a mammalian cell or a yeast cell.

17. The HER2-binding agent of any one of claims 1-13, wherein said HER2-binding agent is conjugated to a label, optionally wherein the label is a fluorescent dye, a radionuclide, an enzyme, a toxin, or a chemotherapeutic agent.

18. The HER2-binding agent of claim 17, wherein the label is a radionuclide.

19. The HER2-binding agent of claim 18, wherein the radionuclide is
a) a radioactive halogen isotope, optionally selected from:
i) ¹⁸F, ⁷⁶Br, ¹²³I, ¹²⁴I, ¹²⁵I, and ¹³¹I; or
ii) ⁷⁵Br, ⁷⁷Br, ¹²²I, ¹²⁴I, ¹²⁵I, ¹³¹I, and ²¹¹At;
b) a radioactive metal isotope, optionally selected from:
i) ⁴⁴Sc, ⁴⁵Ti, ⁵¹Cr, ⁶²Cu, ⁶⁴Cu, ⁶⁶Ga, ⁶⁸Ga, ⁶⁸Ge, ⁷⁵Se, ⁸²Sr, ⁸⁶Y, ⁹⁹Mo, ^{99m}Tc, ^{110m}In, ¹¹¹In, ¹⁶⁶Ho, ¹⁸⁶Re, ^{195m}Pt, and ²⁰¹Tl; or
ii) ⁴⁷Sc, ⁵²Mn, ⁶⁴Cu, ⁶⁷Cu, ⁶⁷Ga, ⁸⁹Zr, ⁹⁰Y, ¹¹¹In, ¹⁵³Sm, ¹⁴⁹Tb, ¹⁶¹Tb, ¹⁶⁶Ho, ¹⁷⁷Lu, ¹⁸⁸Re, ²¹²Pb, ²¹²Bi, ²¹³Bi, ²²⁵Ac, ²²⁶Th, and ²²⁷Th.

20. The HER2-binding agent of any one of claims 1-13 and 17-19, wherein said HER2-binding agent is conjugated to a label via a chelating moiety, optionally wherein the chelating moiety is covalently linked to the protein via a lysine or cysteine residue.

21. The HER2-binding agent of claim 20, wherein the label forms a complex with a metal, and wherein the complex is chelated by the chelating moiety.

22. The HER2-binding agent of any one of claims 1-12 and 17-21, wherein the HER2-binding agent is a targeted radiotherapeutic agent having the structure:
VHH-RLC,
wherein
VHH comprises an amino acid sequence selected from SEQ ID NOs: 1-81 and 205, and
RLC is a radiolabeling chemistry used to directly or indirectly facilitate attachment of a radionuclide to the VHH.

23. The HER2-binding agent of claim 22, wherein the targeted radiotherapeutic agent comprises a prosthetic compound or radiohalogen precursor represented by Formula 2: 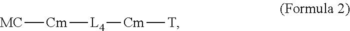
wherein:
MC is a polydentate metal chelating moiety;
Cₘ is thiourea, amide, or thioether;
L4 is selected from a bond, a substituted or unsubstituted alkyl chain, a substituted or unsubstituted alkenyl chain, a substituted or unsubstituted alkynyl chain optionally having NH, CO, or S on one or both termini, and a polyethylene glycol (PEG) chain;
T is the compound of any of:
a) a compound in the form of a prosthetic compound or radiohalogen precursor represented by Formula 1: 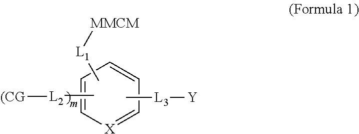
wherein:
X is CH or N;
L₁ and L₃ are independently selected from a bond, a substituted or unsubstituted alkyl chain, a substituted or unsubstituted alkenyl chain, a substituted or unsubstituted alkynyl chain, and a polyethylene glycol (PEG) chain;
MMCM is a macromolecule conjugating moiety;
L₂ is a substituted or unsubstituted alkyl chain, a substituted or unsubstituted alkenyl chain, a substituted or unsubstituted alkynyl chain, or a polyethylene glycol (PEG) chain comprising at least three oxygen atoms, wherein L₂ optionally contains a Brush Border enzyme-cleavable peptide;
CG is selected from guanidine; PO₃H; SO₃H; one or more charged D- or L-amino acids selected from arginine, phosphono/sulfo phenylalanine, glutamate, aspartate, and lysine; a hydrophilic carbohydrate moiety; a polyethylene glycol (PEG) chain; and Z-guanidine;
Z is (CH₂)ₙ;
n is greater than 1;
m is 0 to 3; and
Y is an alkyl metal moiety, boronic acid moiety, boronic ester moiety, or a radioactive halogen selected from the group consisting of ¹⁸F, ⁷⁵Br, ⁷⁶Br, ⁷⁷Br, ¹²²I, ¹²³I, ¹²⁴I, ¹²⁵I, ¹³¹I, and ²¹¹At, or a pharmaceutically acceptable salt or solvate thereof;
b) the compound of a), wherein the compound is a radiohalogen precursor, optionally wherein Y is an alkyl metal moiety selected from the group consisting of trimethyl stannyl (SnMe₃), tri-n-butylstannyl (SnBu₃), trimethylsilyl (SiMe₃), a boronic acid (B(OH)₂), or a boronic ester (B(OR)₂), and optionally wherein R comprises a cyclic moiety or an aliphatic group;
c) the compound of a), wherein the compound is a prosthetic compound, and wherein Y is a radioactive halogen selected from the group consisting of ¹⁸F, ⁷⁵Br, ⁷⁶Br, ⁷⁷Br, ¹²²I, ¹²³I, ¹²⁴I, ¹²⁵I, ¹³¹I and ²¹¹At;
d) the compound of a), wherein MMCM is an active ester or (Gly) m, wherein m is 1 or more;
e) the compound of a), wherein MMCM is selected from the group consisting of N-hydroxysuccinimide (NHS) ester, tetrafluorophenol (TFP) ester, pentafluorophenol (PFP), paranitrolphenol (PNP), an isothiocyanate group, or a maleimide group;
f) the compound of a), wherein MMCM is Gly-Gly-Gly;
g) the compound of a), wherein, wherein L₂ is (CH₂)ₚ, wherein p=1 to 6;
h) the compound of a), wherein the optional Brush Border enzyme-cleavable peptide is selected from the group consisting of Gly-Lys, Gly-Tyr and Gly-Phe-Lys;
i) The compound of a), represented by the following structure: 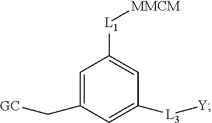
j) the compound of i), wherein the compound comprises N-succinimidyl 3-guanidinomethyl-5-[¹³¹I]iodobenzoate (isoSGMIB), or N-succinimidyl 3-[²¹¹At]astato-5-guanidinomethyl benzoate (isoSAGMB), or maleimidoethyl 2-(guanidinomethyl)-5-iodobenzoate (isoMEGMIB), or maleimidoethyl 3-(guanidinomethyl)-5-astatobenzoate (isoMEGMAB);
or a pharmaceutically acceptable salt or solvate thereof.

24. The HER2-binding agent of claim 23, wherein MC is a macrocyclic structure.

25. The HER2-binding agent of claim 24, wherein
a) MC is selected from DOTA, TETA, NOTP, and NOTA;
b) MC is an acyclic polydentate ligand; or
c) MC is selected from EDTA, EDTMP, and DTPA.

26. The HER2-binding agent of claim 24, wherein the compound is
a) a radiohalogen precursor, optionally wherein Y is an alkyl metal moiety selected from the group consisting of trimethyl stannyl (SnMe₃), tri-n-butylstannyl (SnBu₃), trimethylsilyl (SiMe₃), a boronic acid (B(OH)₂), or a boronic ester (B(OR)₂), and optionally wherein R comprises a cyclic moiety or an aliphatic group; or
b) a prosthetic compound, wherein Y is a radioactive halogen selected from ¹⁸F, ⁷⁵Br, ⁷⁶Br, ⁷⁷Br, ¹²²I, ¹²³I, ¹²⁴I, ¹²⁵I, ¹³¹I, and ²¹¹At.

27. The HER2-binding agent of claim 24, further comprising a metal associated with the MC, optionally wherein the metal is a radioactive metal selected from: ¹⁷⁷Lu, ⁶⁴Cu, ⁶⁷Cu, ¹¹¹In, ⁹⁰Y, ²²⁵Ac, ²¹²Bi, ²¹³Bi, ¹⁵³Sm, ¹⁶⁶Ho ²¹²Pb, ²¹²Bi, ⁶⁷Ga, ⁶⁸Ga ⁸⁹Zr, and ²²⁷Th.

28. A pharmaceutical composition comprising the HER2-binding agent of any one of claims 1-13 and 17-27 and a pharmaceutically acceptable carrier.

29. A method of treating a disease in a subject comprising administering a therapeutically effective amount of the HER2-binding agent of any one of claims 1-13 and 17-27, or the pharmaceutical composition of claim 28, to a subject in need thereof, optionally wherein the disease is cancer.

30. A method of treating cancer comprising administering an effective amount of the HER2-binding agent of any one of claims 1-13 and 17-27, or the pharmaceutical composition of claim 28, to a subject in need thereof.

31. A method of treating cancer comprising administering an effective amount of the targeted radiotherapeutic agent of any one of claims 22-27 to a subject in need thereof.

32. The method of any one of claims 29-31, wherein the cancer is HER2⁺.

33. A method of treating a HER2⁺ cancer comprising administering an effective amount of the HER2-binding agent of any one of claims 1-13 and 17-27, or the pharmaceutical composition of claim 28, to a subject in need thereof.

34. The method of any one of claims 29-33, wherein the cancer is selected from: breast cancer, ovarian cancer, cervical cancer, endometrial cancer, head and neck cancer, brain cancer, gastric cancer, colorectal cancer, colon cancer, pancreatic cancer, renal cancer, prostate cancer, lung cancer, NSCLC, bladder cancer, urothelial cancer, and an epidermal-derived cancer, and/or metastases originating therefrom.

35. The method of any one of claims 29-33, wherein the cancer is breast cancer and/or a metastasis originating therefrom.

36. The method of claim 35, wherein the cancer is a breast cancer brain metastasis.

37. The method of any one of claims 29-36, wherein the subject is scored HER2 (2+) or HER2 (3+) by immunohistochemistry.

38. A method of killing a HER2⁺ cell, comprising contacting the cell with the HER2-binding agent of any one of claims 1-13 and 17-27, or the pharmaceutical composition of claim 28, optionally wherein the cell is a cancer cell.

39. The method of claim 38, wherein the cancer cell is selected from: breast cancer, ovarian cancer, cervical cancer, endometrial cancer, head and neck cancer, brain cancer, gastric cancer, colorectal cancer, colon cancer, pancreatic cancer, renal cancer, prostate cancer, lung cancer, NSCLC, bladder cancer, urothelial cancer, and an epidermal-derived cancer, and/or metastases originating therefrom.

40. The method of claim 38 or 39, wherein the cell is contacted with the HER2-binding agent in vitro or in vivo.

41. A HER2-binding agent of any one of claims 1-13 and 17-27, or a pharmaceutical composition of claim 28, for use in a method of any one of claims 29-40.

42. Use of the HER2-binding agent of any one of claims 1-13 and 17-27, or the pharmaceutical composition of claim 28, in the manufacture of a medicament for the treatment of a disease in a subject in need thereof, optionally wherein the disease is cancer.

43. Use of the HER2-binding agent of any one of claims 1-13 and 17-27, or the pharmaceutical composition of claim 28, in the manufacture of a medicament for use in a method of any one of claims 29-40.

44. A method of diagnosing a disease or medical condition in a subject, comprising administering the HER2-binding agent of any one of claims 1-13 and 17-27, or the pharmaceutical composition of claim 28, to the subject, optionally wherein the disease is cancer.

45. A method of detecting HER2⁺ cells in a subject, comprising:
a) administering a detectably labeled HER2-binding agent of any one of claims 1-13 and 17-27, or a pharmaceutical composition of claim 28 in which the HER2-binding agent has been detectably labeled, to the subject; and
b) detecting binding of the labeled HER2-binding agent to HER2⁺ cells in the subject, wherein the detection of the binding indicates the presence of HER2⁺ cells, optionally wherein detecting binding of the labeled HER2-binding agent to HER2⁺ cells in the subject comprises imaging HER2⁺ cells in the subject.

46. A method of imaging HER2⁺ cells in a subject, comprising administering a detectably labeled HER2-binding agent of any one of claims 1-13 and 17-27, or a pharmaceutical composition of claim 28 in which the HER2-binding agent has been detectably labeled, to the subject, optionally wherein imaging HER2⁺ cells in the subject comprises performing a positron emission tomography (PET) scan or positron emission tomography/computed tomography (PET/CT) scan on the subject.

47. The method of claim 45 or 46, wherein the HER2⁺ cells are HER2⁺ cancer cells.

48. The method of any one of claims 29-37 and 41-47, wherein the subject is a human, non-human primate, or mammal.

49. The method of any one of claims 29-37 and 41-48, wherein the subject has cancer.

50. A method of making a HER2-binding agent, comprising:
a) culturing the host cell of claim 16 under conditions where the HER2-binding agent is produced; and
b) recovering the HER2-binding agent produced by the host cell.


SEQUENCE LISTING (206 sequences)

SEQ ID NO: 1 - protein, 116 aa, synthetic construct
  REGION 1..116: note = Synthetic
QVHLVESGGGLVQPGGSLRLSCVASGFEFSNYDMSWVRQAPGKGLEWVSTISTRGRTTYGYSAKGRFTISRDNAKNTLYLQMDSLESEDTAKYYCNKAIEKSTRPPGQGTQVTVSS

SEQ ID NO: 2 - protein, 120 aa, synthetic construct
  REGION 1..120: note = Synthetic
QVQLVESGGGLVQPGGSLRLSCAASGSIFSINVMGWYRQAPGMQRQLVAIITSGGSTNYADSVKGRFTVSRDNAKNTLYLRMNSLKPEDTAVYYCATIHVRESTWYYAYWGQGTQVTVSS

SEQ ID NO: 3 - protein, 117 aa, synthetic construct
  REGION 1..117: note = Synthetic
QVQLVESGGGLVQAGGSLRLSCAASGRTFSSYAMGWFRQAPGLEREFVAAIARGGVSTGYADSVKGRFAISRDNAKNMVYLQMNSLKPEDTAVYYCATYFWGSPHYWGQGTQVTVSS

SEQ ID NO: 4 - protein, 117 aa, synthetic construct
  REGION 1..117: note = Synthetic
QVQLVESGGGLVQPGGSLRLSCEASGFTFDDYGMSWVRQAPGKGLEWVSGITWNGGNPNYADSVKGRFTISRDNAKRTLYLQMNSLKAEDTAVYYCQRGYSRGYMYWGQGTQVTVSS

SEQ ID NO: 5 - protein, 125 aa, synthetic construct
  REGION 1..125: note = Synthetic
AQVQLVESGGGSVQLGGSLRLSCAVSGLGFSALAMGWFRQAPGKERERVAAISRTGGTTYYTDSVKGRFTISRDNGKNMVYLQMNSLKPEDAAVYYCASRYTALLSTTARGYDYWGQGTQVTVSS

SEQ ID NO: 6 - protein, 118 aa, synthetic construct
  REGION 1..118: note = Synthetic
AQVQLVESGGGLVQSGGSLRLSCAASGIIFRLTTMDWYRQAPGKQRELVATITSGASTNYADSVKGRFTISRDNAKNTMYLQMNSLKAEDTAVYICYARRSYWASDYWGKGTLVTVSS

SEQ ID NO: 7 - protein, 125 aa, synthetic construct
  REGION 1..125: note = Synthetic
AQVQLVESGGGSVQLGGSLRLSCAVSGLDFSALAMGWFRQAPGKERERVAAISRTGGTTYCTDSVKGRFTISRDNGKNMVYLQMNSLKPEDAAVYYCASRYTALLSTTAREYDYWGQGTLVTVSS

SEQ ID NO: 8 - protein, 129 aa, synthetic construct
  REGION 1..129: note = Synthetic
AQVQLVESGGGLVQPGGSLRLSCAASEFTSDNFAIGWFRQAPGKEREGVSCISSDGRMKNYADSVKGRFTISRDNADKAVYLQMNSLKPEDTATYYCAAARVGAEYVCYEAYSAWYDYWGQGTQVTVSS

SEQ ID NO: 9 - protein, 125 aa, synthetic construct
  REGION 1..125: note = Synthetic
AEVQLVESGGGLVQAGGSPRLSCAASGRTFSSYHMGWFRQAPRKEREFVAAITWSGGSTYYADFVKGRFTISRDNAKNTVYLQMNSLKPEDTAVYYCAVGKDFYGIIMRGEYDYWGQGTQVTVSS

SEQ ID NO: 10 - protein, 115 aa, synthetic construct
  REGION 1..115: note = Synthetic
AQVQLVESGGGLVQPGGSLRVSCAASGFPFSSYDMRWVRQAPGKGLEWVSAISPDGRSTFYADSVKGRFTISRDNLKNTLYLQMNSLKPEDTALYYCARGVLDYWGQGTKVTVSS

SEQ ID NO: 11 - protein, 124 aa, synthetic construct
  REGION 1..124: note = Synthetic
AEVQLVESGGGSVQAGGSLRLSCAASGRIFSGWVMGWFREAPGKEREFVAFIRWSSGVATYADSVKGRFDLSIDYAKNTVYLQMNALKPEDTAVYYCNLGIIYSSGTYQPGSSWGQGTLVTVSS

SEQ ID NO: 12 - protein, 122 aa, synthetic construct
  REGION 1..122: note = Synthetic
AQVQLVESGGGLAQPGGSLRLSCAVSGLSLRPLAIGWFRQAPGKPRELVAIVTFSGSTKYASSVKGRFTISRDTAENTVYLQMNSLKPEDTAVYQCSAGRTYGGNTYVADYWGQGTLVTVSS

SEQ ID NO: 13 - protein, 121 aa, synthetic construct
  REGION 1..121: note = Synthetic
AEVQLVESGGGLVQPGGSLRLSCAASEFTFSNYAMSWVRQAPGKGLEWVSAINWSGGITTYADSVKGRFTISRDNAKNTLHLQMNSLKPEDTAVYYCAKGWGTTTRDTGLLSQGTLVTVSS

SEQ ID NO: 14 - protein, 121 aa, synthetic construct
  REGION 1..121: note = Synthetic
AQVQLVESGGGLVQLGGSLRLSCEVSGSVFSILSMGWYRQAPGKQRELVALLSGRETASHESVKGRFTLSRDDASDKVILQLNSLKPEDTAVYYCNLNAREIDVGQYRDYWGQGTQVTVSS

SEQ ID NO: 15 - protein, 119 aa, synthetic construct
  REGION 1..119: note = Synthetic
AQVQLVESGGGLVQAGGSLRLSCTASGRTFSTYNVGWFRQAPGKEREFVAAIMWSGGSTHYADSVKGRFTISRDNAKNMLYLQMNSLKPEDTALYYCVNLPWSRNLEGWGPGTQVTVSS

SEQ ID NO: 16 - protein, 125 aa, synthetic construct
  REGION 1..125: note = Synthetic
AEVQLVESGGGSGQPGGSLSVSCVVSGRADVINVMGWYRQAPGKERELVAAITVGGTYYADAVRGRFTISRDNAKNTLYLQMNSLKPEDTAVYICNMNEYRVATPTTGQQKFEYWGQGTQVTVSS

SEQ ID NO: 17 - protein, 125 aa, synthetic construct
  REGION 1..125: note = Synthetic
AQVQLVESGGGSVQLGGSLRLSCAVSGLGFSALAMGWFRQAPGKERERVAAISRTGGTTYYTDSVKGRFTISRDNGKNMVYLQMNSLKPEDAAVYYCASRYTALLSTTAREYDYWGQGTQVTVSS

SEQ ID NO: 18 - protein, 119 aa, synthetic construct
  REGION 1..119: note = Synthetic
AQVQLVESGGGLVQAGGSLRLSCAASGRTFNSYFYTMAWFRQAPGKEREFVARMNWNADSTYHADSVKGRFTISRENTVYLQMNSLEPEDTAVYYCAAKSISAPKYDYWGQGTQVTVSS

SEQ ID NO: 19 - protein, 129 aa, synthetic construct
  REGION 1..129: note = Synthetic
AQVQLVESGGGLVQPGGSLRLSCAASEFTSDNFAIGWFRQAPGKEREGVSCISSDGRMKNYADSVKGRLTISRDNADKTVYLQMNSLKPEDTATYYCAAARVGAEYVCYEAYSAWYDHWGQGTQVTVSS

SEQ ID NO: 20 - protein, 125 aa, synthetic construct
  REGION 1..125: note = Synthetic
AQVQLVESGGGLVQAGGSPRLSCAASGRTFSSYHMGWFRQAPRKEREFVAAITWSGGSTYYADFVKGRFTISRDNAKNTVYLQMNSLKPEDTAVYYCAVGKDFYGIIMRGEYDYWGQGTLVTVSS

SEQ ID NO: 21 - protein, 113 aa, synthetic construct
  REGION 1..113: note = Synthetic
AEVQLVESGGGLVQAGGSLRLSCAASGSIFGFNVMEWYRQAPGKQRELVAVLGIGTSATYADSVKGRFTISRDNAKNTVYLQMNSLKPEDTAVYYCNAKFVYWGQGTLVTVSS

SEQ ID NO: 22 - protein, 125 aa, synthetic construct
  REGION 1..125: note = Synthetic
AQVQLVESGGGSVQLGGSLRLSCAVSGLGFSALAMGWFRQAPGKERERVAAISRTGGTTYYTDSVKGRFTISRDNGKNMVYLQMNSLKPEDAAVYYCASRYTALLSTTARKYDYWGQGTQVTVSS

SEQ ID NO: 23 - protein, 118 aa, synthetic construct
  REGION 1..118: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCKRFRTAAQGTDYWGQGTQVTVSS

SEQ ID NO: 24 - protein, 118 aa, synthetic construct
  REGION 1..118: note = Synthetic
EVQLVESGGGLVQAGGSLRLSCAASGITFSINTMGWYRQAPGKQRELVALISSIGDTYYADSVKGRFTISRDNAKNTVYLQMNSLKPEDTAVYYCRRFRTAAQGTDYWGQGTQVTVSS

SEQ ID NO: 25 - protein, 118 aa, synthetic construct
  REGION 1..118: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCRRFRTAAQGTDYWGQGTQVTVSS

SEQ ID NO: 26 - protein, 118 aa, synthetic construct
  REGION 1..118: note = Synthetic
EVQLVESGGGLVQAGGSLRLSCAASGITFSINTMGWYRQAPGKQRELVALISSIGDTYYADSVRGRFTISRDNAKNTVYLQMNSLRPEDTAVYYCRRFRTAAQGTDYWGQGTQVTVSS

SEQ ID NO: 27 - protein, 118 aa, synthetic construct
  REGION 1..118: note = Synthetic
EVQLVESGGGLVQAGGSLRLSCAASGITFSINTMGWYRQAPGKQRELVALISSIGDTYYADSVRGRFTISRDNARNTVYLQMNSLKPEDTAVYYCRRFRTAAQGTDYWGQGTQVTVSS

SEQ ID NO: 28 - protein, 118 aa, synthetic construct
  REGION 1..118: note = Synthetic
EVQLVESGGGLVQAGGSLRLSCAASGITFSINTMGWYRQAPGRQRELVALISSIGDTYYADSVRGRFTISRDNAKNTVYLQMNSLKPEDTAVYYCRRFRTAAQGTDYWGQGTQVTVSS

SEQ ID NO: 29 - protein, 118 aa, synthetic construct
  REGION 1..118: note = Synthetic
EVQLVESGGGLVQAGGSLRLSCAASGITFSINTMGWYRQAPGKQRELVALISSIGDTYYADSVRGRFTISRDNARNTVYLQMNSLRPEDTAVYYCRRFRTAAQGTDYWGQGTQVTVSS

SEQ ID NO: 30 - protein, 118 aa, synthetic construct
  REGION 1..118: note = Synthetic
EVQLVESGGGLVQAGGSLRLSCAASGITFSINTMGWYRQAPGRQRELVALISSIGDTYYADSVRGRFTISRDNAKNTVYLQMNSLRPEDTAVYYCRRFRTAAQGTDYWGQGTQVTVSS

SEQ ID NO: 31 - protein, 118 aa, synthetic construct
  REGION 1..118: note = Synthetic
EVQLVESGGGLVQAGGSLRLSCAASGITFSINTMGWYRQAPGRQRELVALISSIGDTYYADSVRGRFTISRDNARNTVYLQMNSLKPEDTAVYYCRRFRTAAQGTDYWGQGTQVTVSS

SEQ ID NO: 32 - protein, 118 aa, synthetic construct
  REGION 1..118: note = Synthetic
EVQLVESGGGLVQAGGSLRLSCAASGITFSINTMGWYRQAPGRQRELVALISSIGDTYYADSVRGRFTISRDNARNTVYLQMNSLRPEDTAVYYCKRFRTAAQGTDYWGQGTQVTVSS

SEQ ID NO: 33 - protein, 118 aa, synthetic construct
  REGION 1..118: note = Synthetic
EVQLVESGGGLVQAGGSLRLSCAASGITFSINTMGWYRQAPGRQRELVALISSIGDTYYADSVKGRFTISRDNARNTVYLQMNSLRPEDTAVYYCRRFRTAAQGTDYWGQGTQVTVSS

SEQ ID NO: 34 - protein, 122 aa, synthetic construct
  REGION 1..122: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCKRFRTAAQGTDYWGQGTQVTVSSGGCG

SEQ ID NO: 35 - protein, 122 aa, synthetic construct
  REGION 1..122: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCKRFRTAAQGTDYWGQGTQVTVSSGGKG

SEQ ID NO: 36 - protein, 121 aa, synthetic construct
  REGION 1..121: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCKRFRTAAQGTDYWGQGTQVTVSSGGC

SEQ ID NO: 37 - protein, 126 aa, synthetic construct
  REGION 1..126: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCKRFRTAAQGTDYWGQGTQVTVSSGGFKGGKG

SEQ ID NO: 38 - protein, 125 aa, synthetic construct
  REGION 1..125: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCKRFRTAAQGTDYWGQGTQVTVSSGGFKGGC

SEQ ID NO: 39 - protein, 121 aa, synthetic construct
  REGION 1..121: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCRRFRTAAQGTDYWGQGTQVTVSSGGC

SEQ ID NO: 40 - protein, 122 aa, synthetic construct
  REGION 1..122: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCRRFRTAAQGTDYWGQGTQVTVSSGGKG

SEQ ID NO: 41 - protein, 126 aa, synthetic construct
  REGION 1..126: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCRRFRTAAQGTDYWGQGTQVTVSSGGFKGGKG

SEQ ID NO: 42 - protein, 125 aa, synthetic construct
  REGION 1..125: note = Synthetic
EVQLVESGGGLVQAGGSLRLSCAASGITFSINTMGWYRQAPGKQRELVALISSIGDTYYADSVRGRFTISRDNAKNTVYLQMNSLKPEDTAVYYCRRFRTAAQGTDYWGQGTQVTVSSGGFKGGC

SEQ ID NO: 43 - protein, 116 aa, synthetic construct
  REGION 1..116: note = Synthetic
QVHLVESGGGLVQPGGSLRLSCVASGFEFSNYDMSWVRQAPGKGLEWVSTISTRGRTTYGYSARGRFTISRDNAKNTLYLQMDSLESEDTAKYYCNKAIEKSTRPPGQGTQVTVSS

SEQ ID NO: 44 - protein, 116 aa, synthetic construct
  REGION 1..116: note = Synthetic
QVHLVESGGGLVQPGGSLRLSCVASGFEFSNYDMSWVRQAPGKGLEWVSTISTRGRTTYGYSAKGRFTISRDNAKNTLYLQMDSLESEDTAKYYCNRAIEKSTRPPGQGTQVTVSS

SEQ ID NO: 45 - protein, 116 aa, synthetic construct
  REGION 1..116: note = Synthetic
QVHLVESGGGLVQPGGSLRLSCVASGFEFSNYDMSWVRQAPGKGLEWVSTISTRGRTTYGYSARGRFTISRDNAKNTLYLQMDSLESEDTAKYYCNRAIEKSTRPPGQGTQVTVSS

SEQ ID NO: 46 - protein, 116 aa, synthetic construct
  REGION 1..116: note = Synthetic
QVHLVESGGGLVQPGGSLRLSCVASGFEFSNYDMSWVRQAPGKGLEWVSTISTRGRTTYGYSAKGRFTISRDNAKNTLYLQMDSLESEDTAKYYCNKAIERSTRPPGQGTQVTVSS

SEQ ID NO: 47 - protein, 116 aa, synthetic construct
  REGION 1..116: note = Synthetic
QVHLVESGGGLVQPGGSLRLSCVASGFEFSNYDMSWVRQAPGKGLEWVSTISTRGRTTYGYSARGRFTISRDNAKNTLYLQMDSLESEDTAKYYCNKAIERSTRPPGQGTQVTVSS

SEQ ID NO: 48 - protein, 116 aa, synthetic construct
  REGION 1..116: note = Synthetic
QVHLVESGGGLVQPGGSLRLSCVASGFEFSNYDMSWVRQAPGKGLEWVSTISTRGRTTYGYSAKGRFTISRDNAKNTLYLQMDSLESEDTAKYYCNRAIERSTRPPGQGTQVTVSS

SEQ ID NO: 49 - protein, 116 aa, synthetic construct
  REGION 1..116: note = Synthetic
QVHLVESGGGLVQPGGSLRLSCVASGFEFSNYDMSWVRQAPGKGLEWVSTISTRGRTTYGYSARGRFTISRDNAKNTLYLQMDSLESEDTAKYYCNRAIERSTRPPGQGTQVTVSS

SEQ ID NO: 50 - protein, 129 aa, synthetic construct
  REGION 1..129: note = Synthetic
AQVQLVESGGGLVQPGGSLRLSCAASEFTSDNFAIGWFRQAPGKEREGVSCISSDGRMRNYADSVKGRFTISRDNADKAVYLQMNSLKPEDTATYYCAAARVGAEYVCYEAYSAWYDYWGQGTQVTVSS

SEQ ID NO: 51 - protein, 129 aa, synthetic construct
  REGION 1..129: note = Synthetic
AQVQLVESGGGLVQPGGSLRLSCAASEFTSDNFAIGWFRQAPGKEREGVSCISSDGRMKNYADSVRGRFTISRDNADKAVYLQMNSLKPEDTATYYCAAARVGAEYVCYEAYSAWYDYWGQGTQVTVSS

SEQ ID NO: 52 - protein, 129 aa, synthetic construct
  REGION 1..129: note = Synthetic
AQVQLVESGGGLVQPGGSLRLSCAASEFTSDNFAIGWFRQAPGKEREGVSCISSDGRMRNYADSVRGRFTISRDNADKAVYLQMNSLKPEDTATYYCAAARVGAEYVCYEAYSAWYDYWGQGTQVTVSS

SEQ ID NO: 53 - protein, 125 aa, synthetic construct
  REGION 1..125: note = Synthetic
AEVQLVESGGGLVQAGGSPRLSCAASGRTFSSYHMGWFRQAPRKEREFVAAITWSGGSTYYADFVRGRFTISRDNAKNTVYLQMNSLKPEDTAVYYCAVGKDFYGIIMRGEYDYWGQGTQVTVSS

SEQ ID NO: 54 - protein, 125 aa, synthetic construct
  REGION 1..125: note = Synthetic
AEVQLVESGGGLVQAGGSPRLSCAASGRTFSSYHMGWFRQAPRKEREFVAAITWSGGSTYYADFVKGRFTISRDNAKNTVYLQMNSLKPEDTAVYYCAVGRDFYGIIMRGEYDYWGQGTQVTVSS

SEQ ID NO: 55 - protein, 125 aa, synthetic construct
  REGION 1..125: note = Synthetic
AEVQLVESGGGLVQAGGSPRLSCAASGRTFSSYHMGWFRQAPRKEREFVAAITWSGGSTYYADFVRGRFTISRDNAKNTVYLQMNSLKPEDTAVYYCAVGRDFYGIIMRGEYDYWGQGTQVTVSS

SEQ ID NO: 56 - protein, 122 aa, synthetic construct
  REGION 1..122: note = Synthetic
AQVQLVESGGGLAQPGGSLRLSCAVSGLSLRPLAIGWFRQAPGKPRELVAIVTFSGSTRYASSVKGRFTISRDTAENTVYLQMNSLKPEDTAVYQCSAGRTYGGNTYVADYWGQGTLVTVSS

SEQ ID NO: 57 - protein, 122 aa, synthetic construct
  REGION 1..122: note = Synthetic
AQVQLVESGGGLAQPGGSLRLSCAVSGLSLRPLAIGWFRQAPGKPRELVAIVTFSGSTKYASSVRGRFTISRDTAENTVYLQMNSLKPEDTAVYQCSAGRTYGGNTYVADYWGQGTLVTVSS

SEQ ID NO: 58 - protein, 122 aa, synthetic construct
  REGION 1..122: note = Synthetic
AQVQLVESGGGLAQPGGSLRLSCAVSGLSLRPLAIGWFRQAPGKPRELVAIVTFSGSTRYASSVRGRFTISRDTAENTVYLQMNSLKPEDTAVYQCSAGRTYGGNTYVADYWGQGTLVTVSS

SEQ ID NO: 59 - protein, 121 aa, synthetic construct
  REGION 1..121: note = Synthetic
AEVQLVESGGGLVQPGGSLRLSCAASEFTFSNYAMSWVRQAPGKGLEWVSAINWSGGITTYADSVRGRFTISRDNAKNTLHLQMNSLKPEDTAVYYCAKGWGTTTRDTGLLSQGTLVTVSS

SEQ ID NO: 60 - protein, 121 aa, synthetic construct
  REGION 1..121: note = Synthetic
AEVQLVESGGGLVQPGGSLRLSCAASEFTFSNYAMSWVRQAPGKGLEWVSAINWSGGITTYADSVKGRFTISRDNAKNTLHLQMNSLKPEDTAVYYCARGWGTTTRDTGLLSQGTLVTVSS

SEQ ID NO: 61 - protein, 121 aa, synthetic construct
  REGION 1..121: note = Synthetic
AEVQLVESGGGLVQPGGSLRLSCAASEFTFSNYAMSWVRQAPGKGLEWVSAINWSGGITTYADSVRGRFTISRDNAKNTLHLQMNSLKPEDTAVYYCARGWGTTTRDTGLLSQGTLVTVSS

SEQ ID NO: 62 - protein, 125 aa, synthetic construct
  REGION 1..125: note = Synthetic
AEVQLVESGGGSGQPGGSLSVSCVVSGRADVINVMGWYRQAPGKERELVAAITVGGTYYADAVRGRFTISRDNAKNTLYLQMNSLKPEDTAVYICNMNEYRVATPTTGQQRFEYWGQGTQVTVSS

SEQ ID NO: 63 - protein, 119 aa, synthetic construct
  REGION 1..119: note = Synthetic
AQVQLVESGGGLVQAGGSLRLSCAASGRTFNSYFYTMAWFRQAPGKEREFVARMNWNADSTYHADSVRGRFTISRENTVYLQMNSLEPEDTAVYYCAAKSISAPKYDYWGQGTQVTVSS

SEQ ID NO: 64 - protein, 119 aa, synthetic construct
  REGION 1..119: note = Synthetic
AQVQLVESGGGLVQAGGSLRLSCAASGRTFNSYFYTMAWFRQAPGKEREFVARMNWNADSTYHADSVKGRFTISRENTVYLQMNSLEPEDTAVYYCAARSISAPKYDYWGQGTQVTVSS

SEQ ID NO: 65 - protein, 119 aa, synthetic construct
  REGION 1..119: note = Synthetic
AQVQLVESGGGLVQAGGSLRLSCAASGRTFNSYFYTMAWFRQAPGKEREFVARMNWNADSTYHADSVRGRFTISRENTVYLQMNSLEPEDTAVYYCAARSISAPKYDYWGQGTQVTVSS

SEQ ID NO: 66 - protein, 119 aa, synthetic construct
  REGION 1..119: note = Synthetic
AQVQLVESGGGLVQAGGSLRLSCAASGRTFNSYFYTMAWFRQAPGKEREFVARMNWNADSTYHADSVKGRFTISRENTVYLQMNSLEPEDTAVYYCAAKSISAPRYDYWGQGTQVTVSS

SEQ ID NO: 67 - protein, 119 aa, synthetic construct
  REGION 1..119: note = Synthetic
AQVQLVESGGGLVQAGGSLRLSCAASGRTFNSYFYTMAWFRQAPGKEREFVARMNWNADSTYHADSVRGRFTISRENTVYLQMNSLEPEDTAVYYCAAKSISAPRYDYWGQGTQVTVSS

SEQ ID NO: 68 - protein, 118 aa, synthetic construct
  REGION 1..118: note = Synthetic
AQVQLVESGGGLVQAGGSLRLSCAASGRTFNSYFYTMAWFRQAPGKEREFVARMNWNADSTYHADSVKGRFTISRENTVYLQMNSLPEDTAVYYCAARSISAPRYDYWGQGTQVTVSS

SEQ ID NO: 69 - protein, 119 aa, synthetic construct
  REGION 1..119: note = Synthetic
AQVQLVESGGGLVQAGGSLRLSCAASGRTFNSYFYTMAWFRQAPGKEREFVARMNWNADSTYHADSVRGRFTISRENTVYLQMNSLEPEDTAVYYCAARSISAPRYDYWGQGTQVTVSS

SEQ ID NO: 70 - protein, 129 aa, synthetic construct
  REGION 1..129: note = Synthetic
AQVQLVESGGGLVQPGGSLRLSCAASEFTSDNFAIGWFRQAPGKEREGVSCISSDGRMRNYADSVKGRLTISRDNADKTVYLQMNSLKPEDTATYYCAAARVGAEYVCYEAYSAWYDHWGQGTQVTVSS

SEQ ID NO: 71 - protein, 129 aa, synthetic construct
  REGION 1..129: note = Synthetic
AQVQLVESGGGLVQPGGSLRLSCAASEFTSDNFAIGWFRQAPGKEREGVSCISSDGRMKNYADSVRGRLTISRDNADKTVYLQMNSLKPEDTATYYCAAARVGAEYVCYEAYSAWYDHWGQGTQVTVSS

SEQ ID NO: 72 - protein, 129 aa, synthetic construct
  REGION 1..129: note = Synthetic
AQVQLVESGGGLVQPGGSLRLSCAASEFTSDNFAIGWFRQAPGKEREGVSCISSDGRMRNYADSVRGRLTISRDNADKTVYLQMNSLKPEDTATYYCAAARVGAEYVCYEAYSAWYDHWGQGTQVTVSS

SEQ ID NO: 73 - protein, 125 aa, synthetic construct
  REGION 1..125: note = Synthetic
AQVQLVESGGGLVQAGGSPRLSCAASGRTFSSYHMGWFRQAPRKEREFVAAITWSGGSTYYADFVRGRFTISRDNAKNTVYLQMNSLKPEDTAVYYCAVGKDFYGIIMRGEYDYWGQGTLVTVSS

SEQ ID NO: 74 - protein, 125 aa, synthetic construct
  REGION 1..125: note = Synthetic
AQVQLVESGGGLVQAGGSPRLSCAASGRTFSSYHMGWFRQAPRKEREFVAAITWSGGSTYYADFVKGRFTISRDNAKNTVYLQMNSLKPEDTAVYYCAVGRDFYGIIMRGEYDYWGQGTLVTVSS

SEQ ID NO: 75 - protein, 125 aa, synthetic construct
  REGION 1..125: note = Synthetic
AQVQLVESGGGLVQAGGSPRLSCAASGRTFSSYHMGWFRQAPRKEREFVAAITWSGGSTYYADFVRGRFTISRDNAKNTVYLQMNSLKPEDTAVYYCAVGRDFYGIIMRGEYDYWGQGTLVTVSS

SEQ ID NO: 76 - protein, 113 aa, synthetic construct
  REGION 1..113: note = Synthetic
AEVQLVESGGGLVQAGGSLRLSCAASGSIFGFNVMEWYRQAPGKQRELVAVLGIGTSATYADSVRGRFTISRDNAKNTVYLQMNSLKPEDTAVYYCNAKFVYWGQGTLVTVSS

SEQ ID NO: 77 - protein, 113 aa, synthetic construct
  REGION 1..113: note = Synthetic
AEVQLVESGGGLVQAGGSLRLSCAASGSIFGFNVMEWYRQAPGKQRELVAVLGIGTSATYADSVKGRFTISRDNAKNTVYLQMNSLKPEDTAVYYCNARFVYWGQGTLVTVSS

SEQ ID NO: 78 - protein, 113 aa, synthetic construct
  REGION 1..113: note = Synthetic
AEVQLVESGGGLVQAGGSLRLSCAASGSIFGFNVMEWYRQAPGKQRELVAVLGIGTSATYADSVRGRFTISRDNAKNTVYLQMNSLKPEDTAVYYCNARFVYWGQGTLVTVSS

SEQ ID NO: 79 - protein, 125 aa, synthetic construct
  REGION 1..125: note = Synthetic
AQVQLVESGGGSVQLGGSLRLSCAVSGLGFSALAMGWFRQAPGKERERVAAISRTGGTTYYTDSVRGRFTISRDNGKNMVYLQMNSLKPEDAAVYYCASRYTALLSTTARKYDYWGQGTQVTVSS

SEQ ID NO: 80 - protein, 125 aa, synthetic construct
  REGION 1..125: note = Synthetic
AQVQLVESGGGSVQLGGSLRLSCAVSGLGFSALAMGWFRQAPGKERERVAAISRTGGTTYYTDSVKGRFTISRDNGKNMVYLQMNSLKPEDAAVYYCASRYTALLSTTARRYDYWGQGTQVTVSS

SEQ ID NO: 81 - protein, 125 aa, synthetic construct
  REGION 1..125: note = Synthetic
AQVQLVESGGGSVQLGGSLRLSCAVSGLGFSALAMGWFRQAPGKERERVAAISRTGGTTYYTDSVRGRFTISRDNGKNMVYLQMNSLKPEDAAVYYCASRYTALLSTTARRYDYWGQGTQVTVSS

SEQ ID NO: 82 - protein, 8 aa, synthetic construct
  REGION 1..8: note = Synthetic
GFEFSNYD

SEQ ID NO: 83 - protein, 8 aa, synthetic construct
  REGION 1..8: note = Synthetic
GSIFSINV

SEQ ID NO: 84 - protein, 8 aa, synthetic construct
  REGION 1..8: note = Synthetic
GRTFSSYA

SEQ ID NO: 85 - protein, 8 aa, synthetic construct
  REGION 1..8: note = Synthetic
GFTFDDYG

SEQ ID NO: 86 - protein, 8 aa, synthetic construct
  REGION 1..8: note = Synthetic
GLGFSALA

SEQ ID NO: 87 - protein, 8 aa, synthetic construct
  REGION 1..8: note = Synthetic
GIIFRLTT

SEQ ID NO: 88 - protein, 8 aa, synthetic construct
  REGION 1..8: note = Synthetic
GLDFSALA

SEQ ID NO: 89 - protein, 8 aa, synthetic construct
  REGION 1..8: note = Synthetic
EFTSDNFA

SEQ ID NO: 90 - protein, 7 aa, synthetic construct
  REGION 1..7: note = Synthetic
GRTFSSY

SEQ ID NO: 91 - protein, 8 aa, synthetic construct
  REGION 1..8: note = Synthetic
GFPFSSYD

SEQ ID NO: 92 - protein, 8 aa, synthetic construct
  REGION 1..8: note = Synthetic
GRIFSGWV

SEQ ID NO: 93 - protein, 8 aa, synthetic construct
  REGION 1..8: note = Synthetic
GLSLRPLA

SEQ ID NO: 94 - protein, 8 aa, synthetic construct
  REGION 1..8: note = Synthetic
EFTFSNYA

SEQ ID NO: 95 - protein, 8 aa, synthetic construct
  REGION 1..8: note = Synthetic
GSVFSILS

SEQ ID NO: 96 - protein, 8 aa, synthetic construct
  REGION 1..8: note = Synthetic
GRTFSTYN

SEQ ID NO: 97 - protein, 8 aa, synthetic construct
  REGION 1..8: note = Synthetic
GRADVINV

SEQ ID NO: 98 - protein, 10 aa, synthetic construct
  REGION 1..10: note = Synthetic
GRTFNSYFYT

SEQ ID NO: 99 - protein, 8 aa, synthetic construct
  REGION 1..8: note = Synthetic
GRTFSSYH

SEQ ID NO: 100 - protein, 8 aa, synthetic construct
  REGION 1..8: note = Synthetic
GSIFGFNV

SEQ ID NO: 101 - protein, 8 aa, synthetic construct
  REGION 1..8: note = Synthetic
GITFSINT

SEQ ID NO: 102 - protein, 7 aa, synthetic construct
  REGION 1..7: note = Synthetic
STRGRTT

SEQ ID NO: 103 - protein, 7 aa, synthetic construct
  REGION 1..7: note = Synthetic
TSGGSTN

SEQ ID NO: 104 - protein, 8 aa, synthetic construct
  REGION 1..8: note = Synthetic
ARGGVSTG

SEQ ID NO: 105 - protein, 8 aa, synthetic construct
  REGION 1..8: note = Synthetic
TWNGGNPN

SEQ ID NO: 106 - protein, 8 aa, synthetic construct
  REGION 1..8: note = Synthetic
SRTGGTTY

SEQ ID NO: 107 - protein, 10 aa, synthetic construct
  REGION 1..10: note = Synthetic
ARMNWNADST

SEQ ID NO: 108 - protein, 8 aa, synthetic construct
  REGION 1..8: note = Synthetic
SSDGRMKN

SEQ ID NO: 109 - protein, 8 aa, synthetic construct
  REGION 1..8: note = Synthetic
TWSGGSTY

SEQ ID NO: 110 - protein, 7 aa, synthetic construct
  REGION 1..7: note = Synthetic
GIGTSAT

SEQ ID NO: 111 - protein, 16 aa, synthetic construct
  REGION 1..16: note = Synthetic
LISSIGDTYYADSVRG

SEQ ID NO: 112 - protein, 8 aa, synthetic construct
  REGION 1..8: note = Synthetic
SSDGRMRN

SEQ ID NO: 113 - protein, 7 aa, synthetic construct
  REGION 1..7: note = Synthetic
TFSGSTR

SEQ ID NO: 114 - protein, 7 aa, synthetic construct
  REGION 1..7: note = Synthetic
TFSGSTK

SEQ ID NO: 115 - protein, 8 aa, synthetic construct
  REGION 1..8: note = Synthetic
NWSGGITT

SEQ ID NO: 116 - protein, 6 aa, synthetic construct
  REGION 1..6: note = Synthetic
TVGGTY

SEQ ID NO: 117 - protein, 7 aa, synthetic construct
  REGION 1..7: note = Synthetic
TSGASTN

SEQ ID NO: 118 - protein, 7 aa, synthetic construct
  REGION 1..7: note = Synthetic
SRTGGTT

SEQ ID NO: 119 - protein, 8 aa, synthetic construct
  REGION 1..8: note = Synthetic
SPDGRSTF

SEQ ID NO: 120 - protein, 8 aa, synthetic construct
  REGION 1..8: note = Synthetic
RWSSGVAT

SEQ ID NO: 121 - protein, 6 aa, synthetic construct
  REGION 1..6: note = Synthetic
SGRETA

SEQ ID NO: 122 - protein, 8 aa, synthetic construct
  REGION 1..8: note = Synthetic
MWSGGSTH

SEQ ID NO: 123 - protein, 6 aa, synthetic construct
  REGION 1..6: note = Synthetic
TVGGTY

SEQ ID NO: 124 - protein, 11 aa, synthetic construct
  REGION 1..11: note = Synthetic
NKAIEKSTRPP

SEQ ID NO: 125 - protein, 14 aa, synthetic construct
  REGION 1..14: note = Synthetic
ATIHVRESTWYYAY

SEQ ID NO: 126 - protein, 10 aa, synthetic construct
  REGION 1..10: note = Synthetic
ATYFWGSPHY

SEQ ID NO: 127 - protein, 10 aa, synthetic construct
  REGION 1..10: note = Synthetic
QRGYSRGYMY

SEQ ID NO: 128 - protein, 17 aa, synthetic construct
  REGION 1..17: note = Synthetic
ASRYTALLSTTARGYDY

SEQ ID NO: 129 - protein, 11 aa, synthetic construct
  REGION 1..11: note = Synthetic
YARRSYWASDY

SEQ ID NO: 130 - protein, 17 aa, synthetic construct
  REGION 1..17: note = Synthetic
ASRYTALLSTTAREYDY

SEQ ID NO: 131 - protein, 20 aa, synthetic construct
  REGION 1..20: note = Synthetic
AAARVGAEYVCYEAYSAWYD

SEQ ID NO: 132 - protein, 17 aa, synthetic construct
  REGION 1..17: note = Synthetic
AVGKDFYGIIMRGEYDY

SEQ ID NO: 133 - protein, 6 aa, synthetic construct
  REGION 1..6: note = Synthetic
NAKFVY

SEQ ID NO: 134 - protein, 17 aa, synthetic construct
  REGION 1..17: note = Synthetic
ASRYTALLSTTARKYDY

SEQ ID NO: 135 - protein, 12 aa, synthetic construct
  REGION 1..12: note = Synthetic
KRFRTAAQGTDY

SEQ ID NO: 136 - protein, 12 aa, synthetic construct
  REGION 1..12: note = Synthetic
RRFRTAAQGTDY

SEQ ID NO: 137 - protein, 7 aa, synthetic construct
  REGION 1..7: note = Synthetic
ARGVLDY

SEQ ID NO: 138 - protein, 16 aa, synthetic construct
  REGION 1..16: note = Synthetic
NLGIIYSSGTYQPGSS

SEQ ID NO: 139 - protein, 13 aa, synthetic construct
  REGION 1..13: note = Synthetic
SAGRTYGGNTYVA

SEQ ID NO: 140 - protein, 13 aa, synthetic construct
  REGION 1..13: note = Synthetic
AKGWGTTTRDTGL

SEQ ID NO: 141 - protein, 15 aa, synthetic construct
  REGION 1..15: note = Synthetic
NLNAREIDVGQYRDY

SEQ ID NO: 142 - protein, 11 aa, synthetic construct
  REGION 1..11: note = Synthetic
VNLPWSRNLEG

SEQ ID NO: 143 - protein, 19 aa, synthetic construct
  REGION 1..19: note = Synthetic
NMNEYRVATPTTGQQKFEY

SEQ ID NO: 144 - protein, 12 aa, synthetic construct
  REGION 1..12: note = Synthetic
AAKSISAPKYDY

SEQ ID NO: 145 - protein, 11 aa, synthetic construct
  REGION 1..11: note = Synthetic
NRAIEKSTRPP

SEQ ID NO: 146 - protein, 11 aa, synthetic construct
  REGION 1..11: note = Synthetic
NKAIERSTRPP

SEQ ID NO: 147 - protein, 11 aa, synthetic construct
  REGION 1..11: note = Synthetic
NRAIERSTRPP

SEQ ID NO: 148 - protein, 17 aa, synthetic construct
  REGION 1..17: note = Synthetic
AVGRDFYGIIMRGEYDY

SEQ ID NO: 149 - protein, 13 aa, synthetic construct
  REGION 1..13: note = Synthetic
ARGWGTTTRDTGL

SEQ ID NO: 150 - protein, 19 aa, synthetic construct
  REGION 1..19: note = Synthetic
NMNEYRVATPTTGQQRFEY

SEQ ID NO: 151 - protein, 12 aa, synthetic construct
  REGION 1..12: note = Synthetic
AARSISAPKYDY

SEQ ID NO: 152 - protein, 12 aa, synthetic construct
  REGION 1..12: note = Synthetic
AAKSISAPRYDY

SEQ ID NO: 153 - protein, 12 aa, synthetic construct
  REGION 1..12: note = Synthetic
AARSISAPRYDY

SEQ ID NO: 154 - protein, 6 aa, synthetic construct
  REGION 1..6: note = Synthetic
NARFVY

SEQ ID NO: 155 - protein, 17 aa, synthetic construct
  REGION 1..17: note = Synthetic
ASRYTALLSTTARRYDY

SEQ ID NO: 156 - protein, 21 aa, synthetic construct
  REGION 1..21: note = Synthetic
AAARVGAEYVCYEAYSAWYDY

SEQ ID NO: 157 - protein, 21 aa, synthetic construct
  REGION 1..21: note = Synthetic
AAARVGAEYVCYEAYSAWYDH

SEQ ID NO: 158 - protein, 13 aa, synthetic construct
  REGION 1..13: note = Synthetic
GRKKRRQRRRPPQ

SEQ ID NO: 159 - protein, 16 aa, synthetic construct
  REGION 1..16: note = Synthetic
RQIKIWFQNRRMKWKK

SEQ ID NO: 160 - protein, 27 aa, synthetic construct
  REGION 1..27: note = Synthetic
GWTLNSAGYLLGKINLKALAALAKKIL

SEQ ID NO: 161 - protein, 18 aa, synthetic construct
  REGION 1..18: note = Synthetic
KLALKLALKALKAALKLA

SEQ ID NO: 162 - protein, 4 aa, synthetic construct
  REGION 1..4: note = Synthetic
RRIP

SEQ ID NO: 163 - protein, 6 aa, synthetic construct
  REGION 1..6: note = Synthetic
NRRPRR

SEQ ID NO: 164 - protein, 21 aa, synthetic construct
  REGION 1..21: note = Synthetic
KETWWETWWTEWSQPKKKRKV

SEQ ID NO: 165 - protein, 12 aa, synthetic construct
  REGION 1..12: note = Synthetic
PLILLRLLRGQF

SEQ ID NO: 166 - protein, 12 aa, synthetic construct
  REGION 1..12: note = Synthetic
PLIYLRLLRGQF

SEQ ID NO: 167 - protein, 15 aa, synthetic construct
  REGION 1..15: note = Synthetic
KLWMRWYSPTTRRYG

SEQ ID NO: 168 - protein, 14 aa, synthetic construct
  REGION 1..14: note = Synthetic
GINTLQKYYCRVRG

SEQ ID NO: 169 - protein, 16 aa, synthetic construct
  REGION 1..16: note = Synthetic
RLWMRWYSPRTRAYGC

SEQ ID NO: 170 - protein, 15 aa, synthetic construct
  REGION 1..15: note = Synthetic
GKCSTRGRKCCRRKK

SEQ ID NO: 171 - protein, 14 aa, synthetic construct
  REGION 1..14: note = Synthetic
GRCAVLSCLPKEQI

SEQ ID NO: 172 - protein, 6 aa, synthetic construct
  REGION 1..6: note = Synthetic
GGSGGS

SEQ ID NO: 173 - protein, 9 aa, synthetic construct
  REGION 1..9: note = Synthetic
GGSGGSGGS

SEQ ID NO: 174 - protein, 12 aa, synthetic construct
  REGION 1..12: note = Synthetic
GGSGGSGGSGGS

SEQ ID NO: 175 - protein, 15 aa, synthetic construct
  REGION 1..15: note = Synthetic
GGSGGSGGSGGSGGS

SEQ ID NO: 176 - protein, 4 aa, synthetic construct
  REGION 1..4: note = Synthetic
GGGG

SEQ ID NO: 177 - protein, 5 aa, synthetic construct
  REGION 1..5: note = Synthetic
GGGGG

SEQ ID NO: 178 - protein, 6 aa, synthetic construct
  REGION 1..6: note = Synthetic
GGGGGG

SEQ ID NO: 179 - protein, 25 aa, synthetic construct
  REGION 1..25: note = Synthetic
  VARIANT 6..25: note = "GGGGS" may or may not be present
GGGGSGGGGSGGGGSGGGGSGGGGS

SEQ ID NO: 180 - protein, 24 aa, synthetic construct
  REGION 1..24: note = Synthetic
  VARIANT 7..24: note = "GGGGGS" may or may not be present
GGGGGSGGGGGSGGGGGSGGGGGS

SEQ ID NO: 181 - protein, 5 aa, synthetic construct
  REGION 1..5: note = Synthetic
GGGGS

SEQ ID NO: 182 - protein, 6 aa, synthetic construct
  REGION 1..6: note = Synthetic
GGGGGS

SEQ ID NO: 183 - protein, 18 aa, synthetic construct
  REGION 1..18: note = Synthetic
GGGGGSGGGGGSGGGGGS

SEQ ID NO: 184 - protein, 15 aa, synthetic construct
  REGION 1..15: note = Synthetic
GGGGSGGGGSGGGGS

SEQ ID NO: 185 - protein, 18 aa, synthetic construct
  REGION 1..18: note = Synthetic
GGSGGGGSGGGGSGGGGS

SEQ ID NO: 186 - protein, 5 aa, synthetic construct
  REGION 1..5: note = Synthetic
PGGGG

SEQ ID NO: 187 - protein, 64 aa, synthetic construct
  REGION 1..64: note = Synthetic
  VARIANT 5..64: note = "GGGS" may or may not be present
GGGSGGGSGGGSGGGSGGGSGGGSGGGSGGGSGGGSGGGSGGGSGGGSGGGSGGGSGGGSGGGS

SEQ ID NO: 188 - protein, 80 aa, synthetic construct
  REGION 1..80: note = Synthetic
  VARIANT 6..80: note = "GGGGS" may or may not be present
GGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 189 - protein, 15 aa, synthetic construct
  REGION 1..15: note = Synthetic
GGGGSGGGGSGGGGS

SEQ ID NO: 190 - protein, 56 aa, synthetic construct
  REGION 1..56: note = Synthetic
  VARIANT 15..56: note = "EGKSSGSGSESKST" may or may not be present
EGKSSGSGSESKSTEGKSSGSGSESKSTEGKSSGSGSESKSTEGKSSGSGSESKST

SEQ ID NO: 191 - protein, 72 aa, synthetic construct
  REGION 1..72: note = Synthetic
  VARIANT 19..72: note = "KESGSVSSEQLAQFRSLD" may or may not be present
KESGSVSSEQLAQFRSLDKESGSVSSEQLAQFRSLDKESGSVSSEQLAQFRSLDKESGSVSSEQLAQFRSLD

SEQ ID NO: 192 - protein, 14 aa, synthetic construct
  REGION 1..14: note = Synthetic
GGGSLVPRGSGGGS

SEQ ID NO: 193 - protein, 55 aa, synthetic construct
  REGION 1..55: note = Synthetic
  VARIANT 12..55: note = "GAGSAAGSGEF" may or may not be present
GAGSAAGSGEFGAGSAAGSGEFGAGSAAGSGEFGAGSAAGSGEFGAGSAAGSGEF

SEQ ID NO: 194 - protein, 96 aa, synthetic construct
  REGION 1..96: note = Synthetic
  VARIANT 7..96: note = "GAGAGX" may or may not be present
GAGAGXGAGAGXGAGAGXGAGAGXGAGAGXGAGAGXGAGAGXGAGAGXGAGAGXGAGAGXGAGAGXGAGAGXGAGAGXGAGAGXGAGAGXGAGAGX

SEQ ID NO: 195 - protein, 96 aa, synthetic construct; identical to SEQ ID NO: 194
  REGION 1..96: note = Synthetic
  VARIANT 1..96: note = Every X may be A, Y, V or S
  VARIANT 7..96: note = "GAGAGX" may or may not be present

SEQ ID NO: 196 - protein, 200 aa, synthetic construct
  REGION 1..200: note = Synthetic
  VARIANT 1..200: note = Every X may be A, V, G or S
  VARIANT 6..200: note = "VPGXG" may or may not be present
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 197 - protein, 136 aa, synthetic construct
  REGION 1..136: note = Synthetic
  VARIANT 41..136: note = "APAP" may or may not be present
APAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAPAP

SEQ ID NO: 198 - protein, 15 aa, synthetic construct
  REGION 1..15: note = Synthetic
  VARIANT 6..15: note = "EAAAK" may or may not be present
EAAAKEAAAKEAAAK

SEQ ID NO: 199 - protein, 46 aa, synthetic construct
  REGION 1..46: note = Synthetic
AEAAAKEAAAKEAAAKEAAAKALEAEAAAKEAAAKEAAAKEAAAKA

SEQ ID NO: 200 - protein, 9 aa, synthetic construct
  REGION 1..9: note = Synthetic
AAAHHHHHH

SEQ ID NO: 201 - protein, 4 aa, synthetic construct
  REGION 1..4: note = Synthetic
GGCG

SEQ ID NO: 202 - protein, 4 aa, synthetic construct
  REGION 1..4: note = Synthetic
GGKG

SEQ ID NO: 203 - protein, 7 aa, synthetic construct
  REGION 1..7: note = Synthetic
GGKGGKG

SEQ ID NO: 204 - protein, 6 aa, synthetic construct
  REGION 1..6: note = Synthetic
GGKGGC

SEQ ID NO: 205 - protein, 118 aa, synthetic construct
  REGION 1..118: note = Synthetic
EVQLVESGGGLVQAGGSLRLSCAASGITFSINTMGWYRQAPGRQRELVALISSIGDTYYADSVRGRFTISRDNARNTVYLQMNSLRPEDTAVYYCRRFRTAAQGTDYWGQGTQVTVSS

SEQ ID NO: 206 - protein, 15 aa, synthetic construct
  REGION 1..15: note = Synthetic
SAGRTYGGNTYVADY